(19) United States
(12) Patent Application Publication
(10) Pub. No.: US 2024/0301435 A1
(43) Pub. Date: Sep. 12, 2024

(54) COMPOSITIONS AND METHODS FOR TRANSFORMATION OF MONOCOT SEED EXCISED EMBRYO EXPLANTS

(71) Applicant: Monsanto Technology LLC, St. Louis, MO (US)

(72) Inventors: Justin Arsenault, Ivoryton, CT (US); Yurong Chen, Chesterfield, MO (US); Byung-Guk Kang, Chesterfield, MO (US); Jennifer Kumpf, North Stonington, CT (US); Brian J. Martinell, Mt. Horeb, WI (US); Lorena B. Moeller, Wildwood, MO (US); Mary Ann Saltarikos, O'Fallon, MO (US); Ashok Shrawat, Chesterfield, MO (US); Shubha Subbarao, Chesterfield, MO (US); Edward J. Williams, Madison, WI (US); Xudong Ye, Chesterfield, MO (US)

(21) Appl. No.: 18/609,293

(22) Filed: Mar. 19, 2024

Related U.S. Application Data
(63) Continuation-in-part of application No. PCT/US22/77024, filed on Sep. 26, 2022.
(63) Continuation-in-part of application No. PCT/US22/77015, filed on Sep. 26, 2022.
(63) Continuation-in-part of application No. PCT/US22/77027, filed on Sep. 26, 2022.
(63) Continuation-in-part of application No. PCT/US22/77018, filed on Sep. 26, 2022.
(63) Continuation-in-part of application No. PCT/US22/77031, filed on Sep. 26, 2022.
(60) Provisional application No. 63/248,921, filed on Sep. 27, 2021.
(60) Provisional application No. 63/248,928, filed on Sep. 27, 2021.
(60) Provisional application No. 63/248,970, filed on Sep. 27, 2021.
(60) Provisional application No. 63/248,945, filed on Sep. 27, 2021.
(60) Provisional application No. 63/248,960, filed on Sep. 27, 2021.

(30) Foreign Application Priority Data
Oct. 20, 2022 (DE) 10 2022 127 658.9

(51) Int. Cl. C12N 15/82; A01H 4/00

(52) U.S. Cl. CPC C12N 15/8205; A01H 4/008

(57) ABSTRACT

The invention provides novel compositions and methods for improving the transformation of monocot seed excised embryo explants, which may include one or more steps of explant preparation, explant rehydration, Rhizobiales bacterium inoculation and co-culture, bud induction, extended bud induction, or regeneration of genetically modified plants or plant parts. The methods provided herein may include transforming at least one plant cell of the embryo explant with a heterologous polynucleotide by inoculating the embryo explant with a Rhizobiales bacterium comprising the heterologous polynucleotide. The methods provided herein also include methods of regenerating a genetically modified plant or plant part from a transformed or edited plant cell or explant.

### CROSS-REFERENCE TO RELATED APPLICATIONS

This application is a continuation-in-part of PCT International Appl. No. PCT/US22/77024 filed Sep. 26, 2022, which claims the priority of U.S. Provisional Appl. Ser. No. 63/248,921 filed Sep. 27, 2021. This application is a continuation-in-part of PCT International Appl. No. PCT/US22/77015 filed Sep. 26, 2022, which claims the priority of U.S. Provisional Appl. Ser. No. 63/248,928 filed Sep. 27, 2021. This application is a continuation-in-part of PCT International Appl. No. PCT/US22/77027 filed Sep. 26, 2022, which claims the priority of U.S. Provisional Appl. Ser. No. 63/248,970 filed Sep. 27, 2021. This application is a continuation-in-part of PCT International Appl. No. PCT/US22/77018 filed Sep. 26, 2022, which claims the priority of U.S. Provisional Appl. Ser. No. 63/248,945 filed Sep. 27, 2021. This application is a continuation-in-part of PCT International Appl. No. PCT/US22/77031 filed Sep. 26, 2022, which claims the priority of U.S. Provisional Appl. Ser. No. 63/248,960 filed Sep. 27, 2021. The entire disclosure of each of which is incorporated herein by reference in its entirety.

### FIELD OF THE INVENTION

The present disclosure relates to compositions and methods for improving the genetic modification of monocot seed excised embryo explants.

### BACKGROUND OF THE INVENTION

Monocot plants, such as corn, wheat, rice, barley, and sorghum, are important crops and are primary food sources in many areas of the world. Biotechnology methods have been used to improve these crops by the creation of novel traits through genetic modifications of plants, which often rely on the delivery of polynucleotide molecules to plant cells to produce genetically modified plants or plant parts having the improved traits or characteristics. However, there is a continuing need in the art for improved methods of genetically modifying a plant, particularly a monocot plant, that do not rely on the use of callus culture, that can be performed more efficiently, and that are less plant germplasm dependent.

The disclosure described herein provide novel compositions and methods for the improved transformation of monocot seed excised embryo explants and the regeneration of genetically modified plants or plant parts therefrom without the use of callus culture that overcome many of the challenges and limitations in the art.

### SUMMARY OF THE INVENTION

In one aspect, the present disclosure provides a method of producing a genetically modified monocot plant or plant part comprising: introducing a heterologous polynucleotide molecule into at least one cell of a monocot seed embryo explant comprising meristematic tissue by inoculating the embryo explant with an inoculation medium comprising a Rhizobiales bacterium competent to transform the at least one cell with the heterologous polynucleotide molecule; culturing the embryo explant in contact with a first bud induction medium comprising a first auxin and a first cytokinin at a temperature of about 33° C. to about 37° C. for a first time period followed by culturing the embryo explant in contact with the first bud induction medium at a temperature of about 26° C. to about 30° C. for a second time period, wherein the concentration of the first cytokinin in the first bud induction medium is about 5 mg/L to about 15 mg/L and the concentration of the first auxin in the first bud induction medium is about 0.5 mg/L to about 1.5 mg/L; and regenerating the genetically modified monocot plant or plant part from the embryo explant. In one embodiment, the first time period or the second time period is about 2 days to about 14 days or about 6 days to about 8 days. In another embodiment, the first cytokinin is selected from the group consisting of: 6-benzylaminopurine (BAP), thidiazuron (TDZ), kinetin, zeatin, diphenyl urea (DPU), 6-(gamma,gamma-dimethylallylamino)purine (2iP), and meta-topolin. In yet another embodiment, the first auxin is selected from the group consisting of: 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba). In still yet another embodiment, the first cytokinin is 6-benzylaminopurine (BAP) and the first auxin is 2,4-dichlorophenoxy-acetic acid (2,4-D). The method, in one embodiment, may further comprise culturing the embryo explant in contact with a second bud induction medium comprising the first auxin or a second auxin and the first cytokinin or a second cytokinin, wherein the culturing of the embryo explant in contact with the second bud induction medium is performed after the embryo explant is cultured in contact with the first bud induction medium and before regenerating the genetically modified monocot plant or plant part from the cultured embryo explant in contact with the regeneration medium. The embryo explant, in another embodiment, is cultured in contact with the second bud induction medium for about 4 days to about 28 days or about 7 to about 14 days. The embryo explant, in still yet another embodiment, is cultured in contact with the second bud induction medium at a temperature of about 20° C. to about 32° C., about 25° C. to about 29° C., or about 27° C. to about 28° C. In one embodiment, the second bud induction medium comprises a high cytokinin to auxin ratio. In another embodiment, the genetically modified monocot plant is a corn plant, a wheat plant, a rice plant, a barley plant, a turfgrass plant, or a sorghum plant. In yet another embodiment, the method may further comprise co-culturing the embryo explant with the Rhizobiales bacterium in contact with a co-culture medium. In still yet another embodiment, the method may further comprise applying a force treatment to the embryo explant in the inoculation medium or prior to inoculating the embryo explant with the inoculation medium.

In another aspect, the present disclosure provides a method of producing a genetically modified monocot plant or plant part comprising: introducing a heterologous polynucleotide molecule into at least one cell of a monocot seed embryo explant comprising meristematic tissue by inoculating the embryo explant with an inoculation medium comprising a Rhizobiales bacterium competent to transform the at least one cell with the heterologous polynucleotide molecule; applying a pressure treatment and a gravitational force treatment to the embryo explant; culturing the embryo explant in contact with a first bud induction medium comprising a first auxin and a first cytokinin at a temperature of about 33° C. to about 37° C.; and regenerating the genetically modified monocot plant or plant part from the embryo explant. The method may comprise, in one embodiment, applying the pressure treatment and the gravitational force treatment to the embryo explant in contact with the inoculation medium. The method may comprise, in another embodiment, applying the pressure treatment and the gravitational force treatment to the embryo explant prior to inoculating the embryo explant with the inoculation medium. The method may comprise, in yet another embodiment, applying the pressure treatment before the gravitational force treatment. The method may comprise, in still yet another embodiment, applying the gravitational force treatment before the pressure treatment. In one embodiment, the method may comprise applying the gravitational force treatment to the embryo explant for about 1 minute to about 2 hours, about 2 minutes to about 110 minutes, about 5 minutes to about 90 minutes, about 10 minutes to about 60 minutes, or about 20 minutes to about 40 minutes. In another embodiment, the method may comprise applying the pressure treatment to the embryo explant for about 10 seconds to about 10 minutes, about 15 seconds to about 8 minutes, about 30 seconds to about 6 minutes, or about 2 minutes to about 4 minutes. In yet another embodiment, applying the gravitational force treatment comprises applying about 100×g to about 10,000×g, about 500×g to about 3,000×g, about 655×g, or about 2620×g of force. In still yet another embodiment, applying the pressure treatment comprises applying about 100 psi to about 1,000 psi, about 125 psi to about 750 psi, about 150 psi to about 500 psi, about 200 psi to about 400 psi, or about 300 psi of force. Non-limiting examples of Rhizobiales bacterium that may be used according to certain embodiments of the present disclosure include a Rhizobiaceae bacterium, a Phyllobacteriaceae bacterium, a Brucellaceae bacterium, a Bradyrhizobiaceae bacterium, a Xanthobacteraceae bacterium, an Agrobacterium bacterium, a Rhizobium bacterium, a Sinorhizobium bacterium, a Mesorhizobium bacterium, a Phyllobacterium bacterium, an Ochrobactrum bacterium, a Bradyrhizobium bacterium, and an Azorhizobium bacterium. The OD₆₆₀ of Rhizobiales bacterium in the inoculation medium, in one embodiment, is about 0.5 to about 2.0, about 0.75 to about 1.25, or about 1.0. In another embodiment, the methods of the present disclosure may further comprise co-culturing the embryo explant with the Rhizobiales bacterium in contact with a co-culture medium. The OD₆₆₀ of the Rhizobiales bacterium in the co-culture medium, in yet another embodiment, is about 0.5 to about 2.0, about 0.75 to about 1.25, or about 1.0. In still yet another embodiment, the genetically modified monocot plant is a corn plant, a wheat plant, a rice plant, a barley plant, a turfgrass plant, or a sorghum plant.

In yet another aspect, the present disclosure provides a method of producing a genetically modified monocot plant or plant part comprising: introducing a heterologous polynucleotide molecule into at least one cell of a monocot seed embryo explant comprising meristematic tissue; culturing the embryo explant in contact with a first bud induction medium comprising a first auxin and a first cytokinin; and regenerating the genetically modified monocot plant or plant part from the cultured embryo explant in contact with a regeneration medium, wherein the regeneration medium comprises a total nitrogen concentration of about 0.5 mM to about 20 mM. In one embodiment, the regeneration medium comprises a total salt concentration of less than about 2800 mg/L, less than about 2500 mg/L, or in a range from about 2200 mg/L to about 2500 mg/L. In another embodiment, the regeneration medium comprises a nitrate ion concentration of about 0.5 mM to about 20 mM. In yet another embodiment, the regeneration medium comprises an ammonium ion concentration of about 0.5 mM to about 15 mM. In still yet another embodiment, the regeneration medium comprises a potassium ion concentration of about 0.5 mM to about 15 mM. The sulfate ion concentration in the regeneration medium, in one embodiment, is greater than or equal to about 5 mM. The ammonium nitrate concentration in the regeneration medium, in another embodiment, is about 100 mg/L to about 1000 mg/L. The calcium chloride concentration in the regeneration medium, in yet another embodiment, is less than or equal to about 100 mg/L. The calcium nitrate concentration in the regeneration medium, in still yet another embodiment, is less than or equal to about 500 mg/L. In one embodiment, the regeneration medium comprises a potassium sulfate concentration greater than or equal to about 500 mg/L. In another embodiment, the methods of the present disclosure may comprise regenerating the genetically modified monocot plant or plant part at a temperature of about 20° C. to about 32° C., about 25° C. to about 29° C., or about 27° C. to about 28° C. In yet another embodiment, the methods of the present disclosure may comprise regenerating the genetically modified monocot plant or plant part for about 20 days to about 50 days or about 28 days to about 42 days. In still yet another embodiment, the methods of the present disclosure may further comprise culturing the embryo explant in contact with a second bud induction medium comprising the first auxin or a second auxin and the first cytokinin or a second cytokinin, wherein the culturing of the embryo explant in contact with the second bud induction medium is performed after the embryo explant is cultured in contact with the first bud induction medium and before regenerating the genetically modified monocot plant or plant part from the cultured embryo explant in contact with the regeneration medium. In one embodiment, the methods of the present disclosure may further comprise: co-culturing the embryo explant with the Rhizobiales bacterium in contact with a co-culture medium; or applying a force treatment to the embryo explant in the inoculation medium or prior to inoculating the embryo explant with the inoculation medium. In another embodiment, the genetically modified monocot plant is a corn plant, a wheat plant, a rice plant, a barley plant, a turfgrass plant, or a sorghum plant.

In still yet another aspect, the present disclosure provides a method of producing a genetically modified monocot plant or plant part comprising: culturing the monocot seed embryo explant in contact with a first bud induction medium comprising a first auxin and a first cytokinin, wherein the first bud induction medium comprises a high cytokinin to auxin ratio, culturing the monocot seed embryo explant in contact with a second bud induction medium comprising the first auxin or a second auxin, and the first cytokinin or a second cytokinin, and regenerating the genetically modified monocot plant or plant part from the cultured monocot seed embryo explant in contact with a regeneration medium. In some embodiments, the plant part is a shoot or a root.

In a further aspect, the present disclosure provides a method of producing a genetically modified monocot plant or plant part comprising: introducing a heterologous polynucleotide molecule into at least one cell of a monocot seed embryo explant comprising meristematic tissue by inoculating the embryo explant with an inoculation medium comprising a Rhizobiales bacterium competent to transform the at least one cell with the heterologous polynucleotide molecule, co-culturing the embryo explant with the Rhizobiales bacterium in contact with a co-culture medium, culturing the embryo explant in contact with a first bud induction medium comprising a first auxin and a first cytokinin, culturing the embryo explant in contact with a second bud induction medium comprising the first auxin or a second auxin and the first cytokinin or a second cytokinin, and regenerating the genetically modified monocot plant or plant part from the embryo explant. In one embodiment, the plant part is a shoot or a root. In another embodiment, the genetically modified monocot plant is a corn plant, a wheat plant, a rice plant, a barley plant, a turfgrass plant, or a sorghum plant. In yet another embodiment, the genetically modified monocot plant is a corn plant. In still yet another embodiment, the genetically modified monocot plant is a wheat plant. In certain embodiments, the Rhizobiales bacterium is selected from the group consisting of a) a Rhizobiaceae, a Phyllobacteriaceae, a Brucellaceae, a Bradyrhizobiaceae, and a Xanthobacteraceae bacterium; or b) an Agrobacterium, a Rhizobium, a Sinorhizobium, a Mesorhizobium, a Phyllobacterium, an Ochrobactrum, a Bradyrhizobium, and an Azorhizobium bacterium. In one embodiment, the Rhizobiales bacterium is Agrobacterium, and the OD₆₆₀ of Agrobacterium in the inoculation medium is about 0.5 to about 2.0. In certain embodiments, the Rhizobiales bacterium is Agrobacterium, and the OD₆₆₀ of Agrobacterium in the inoculation medium is about 0.75 to about 1.25 or is about 1.0. In another embodiment, the genetically modified monocot plant or plant part is non-chimeric. The embryo explant and the genetically modified monocot plant or plant part, in yet another embodiment, are cultured and regenerated without producing a callus tissue culture. In still yet another embodiment, the heterologous polynucleotide molecule comprises a gene of interest or one or more expression cassettes encoding a guide RNA or a site-directed nuclease. In particular embodiments, the present disclosure provides a method of producing a genetically modified monocot plant or plant part further comprising applying a force treatment to the embryo explant in the inoculation medium.

In one aspect, the present disclosure provides a method of co-culturing an embryo explant with a Rhizobiales bacterium. In one embodiment, the embryo explant is co-cultured at a temperature of about 15° C. to about 25° C. or about 20° C. In another embodiment, the embryo explant is co-cultured for a time period in a range from about 2 days to about 10 days or from about 5 days to about 7 days. In yet another embodiment, the co-culture medium does not contain an auxin or a cytokinin. In still yet another embodiment, the co-culture medium does not contain a surfactant. In one embodiment, the co-culture medium is in contact with a paper substrate wetted with the co-culture medium. In another embodiment, the co-culture medium comprises a second Rhizobiales bacterium competent to transform the at least one cell with the heterologous polynucleotide molecule. In yet another embodiment, the Rhizobiales bacterium and the second Rhizobiales bacterium are of the same species. In yet another embodiment, the second Rhizobiales bacterium is Agrobacterium, and the OD₆₆₀ of Agrobacterium in the co-culture medium is about 0.5 to about 2.0. In still yet another embodiment, the second Rhizobiales bacterium is Agrobacterium, and the OD₆₆₀ of Agrobacterium in the co-culture medium is about 0.75 to about 1.25 or is about 1.0.

In another aspect, the disclosure provides methods of producing a genetically modified monocot plant or plant part comprising: introducing a heterologous polynucleotide molecule into at least one cell of a monocot seed embryo explant comprising meristematic tissue, culturing the monocot seed embryo explant in contact with a bud induction medium comprising an auxin and a cytokinin, and regenerating the genetically modified monocot plant or plant part from the cultured monocot seed embryo explant in contact with a regeneration medium, wherein the monocot seed embryo explant is cultured in contact with the bud induction medium at an elevated temperature in a range from about 30° C. to about 40° C. In certain embodiments, the elevated temperature is about 30° C. to about 37° C. In other embodiments, the elevated temperature is about 30° C. to about 35° C. In yet other embodiments, the elevated temperature is about 33° C. to about 35° C.

In yet another aspect, the present disclosure provides a method of producing a genetically modified monocot plant or plant part comprising: introducing a heterologous polynucleotide molecule into at least one cell of a monocot seed embryo explant comprising meristematic tissue by inoculating the embryo explant with an inoculation medium comprising a Rhizobiales bacterium competent to transform the at least one cell with the heterologous polynucleotide molecule, wherein a force treatment is applied to the embryo explant in contact with the inoculation medium, culturing the embryo explant in contact with a first bud induction medium comprising a first auxin and a first cytokinin, and regenerating the genetically modified monocot plant or plant part from the embryo explant. In another aspect, the present disclosure provides, a method of producing a genetically modified monocot plant or plant part comprising: applying a force treatment to a monocot seed embryo explant comprising meristematic tissue, introducing a heterologous polynucleotide molecule into at least one cell of the embryo explant by inoculating the embryo explant with an inoculation medium comprising a Rhizobiales bacterium competent to transform the at least one cell with the heterologous polynucleotide molecule, culturing the embryo explant in contact with a first bud induction medium comprising a first auxin and a first cytokinin, and regenerating the genetically modified monocot plant or plant part from the embryo explant. Non-limiting examples of monocot plants which may be used according to the present disclosure include corn plants, wheat plants, rice plants, barley plants, turfgrass plants, and sorghum plants. In certain embodiments, the Rhizobiales bacterium is selected from the group consisting of a Rhizobiaceae, a Phyllobacteriaceae, a Brucellaceae, a Bradyrhizobiaceae, a Xanthobacteraceae, an Agrobacterium, a Rhizobium, a Sinorhizobium, a Mesorhizobium, a Phyllobacterium, an Ochrobactrum, a Bradyrhizobium, and an Azorhizobium bacterium. In particular embodiments, the Rhizobiales bacterium is Agrobacterium, and the OD₆₆₀ of Agrobacterium in the inoculation medium is about 0.5 to about 2.0. The OD₆₆₀ of Agrobacterium in the inoculation medium, in some embodiments, is about 0.75 to about 1.25, or is about 1.0. In another embodiment, the genetically modified plant part is a shoot or a root. In yet another embodiment, the inoculation medium is a liquid medium. In particular embodiments, the embryo explant is submerged in the inoculation medium when the force treatment is applied, or the force treatment is applied to the embryo explant after an excess amount of the inoculation medium is removed. In yet another embodiment, the heterologous polynucleotide molecule comprises a selectable marker gene providing resistance to a selection agent. Non-limiting examples of selection agents include kanamycin, paromomycin, hygromycin B, spectinomycin, streptomycin, gentamycin, glyphosate, glufosinate, phosphinothricin, bromoxynil, bialaphos, dicamba, imidazolinone, and sulfonylurea. In still yet another embodiment, the heterologous polynucleotide molecule comprises a gene of interest or one or more expression cassettes encoding a guide RNA or a site-directed nuclease.

In still yet another aspect, the present disclosure provides a force treatment, which may be applied to an embryo explant either prior to, during, or prior to and during inoculation with a Rhizobiales bacterium. Non-limiting examples of a force treatment which may be applied to an embryo explant include a pressure treatment and a gravitational force treatment. In one embodiment, the pressure treatment comprises applying about 100 psi to about 20,000 psi, about 100 psi to about 1,000 psi, about 125 psi to about 750 psi, about 150 psi to about 500 psi, about 200 psi to about 400 psi, or about 300 psi of pressure. The pressure treatment is applied, in another embodiment, for about 10 seconds to about 10 minutes, about 15 seconds to about 8 minutes, about 30 seconds to about 6 minutes, about 2 minutes to about 4 minutes, or about 3 minutes. In yet another embodiment, the gravitational force treatment comprises applying about 100×g to about 10,000×g, about 500×g to about 3,000×g, about 655×g, or about 2620×g of gravitational force. The gravitational force treatment is applied, in still yet another embodiment, for about 1 minute to about 2 hours, about 2 minutes to about 110 minutes, about 5 minutes to about 90 minutes, about 10 minutes to about 60 minutes, about 20 minutes to about 40 minutes, or about 30 minutes. In one embodiment, a force treatment for use according to the present disclosure comprises applying a pressure treatment and a gravitational force treatment to the embryo explant. The pressure treatment is applied, in particular embodiments, before the gravitational force treatment or after the gravitational force treatment.

In a further aspect, the present disclosure provides a method comprising applying a vacuum treatment to the embryo explant. The vacuum treatment is applied, in certain embodiments, before a force treatment or after a force treatment. In one embodiment, where the force treatment comprises applying both a pressure treatment and a gravitational force treatment, the vacuum treatment is applied, for example, after the pressure treatment and before the gravitational force treatment or after the gravitational force treatment and before the pressure treatment. In another embodiment, the vacuum treatment comprises applying about 0.05 atm to about 0.50 atm of pressure.

In one aspect, the present disclosure provides a method of producing a genetically modified monocot plant cell comprising: transforming at least one cell of a monocot seed embryo explant comprising meristematic tissue with a heterologous polynucleotide molecule by inoculating the embryo explant with an inoculation medium comprising a Rhizobiales bacterium competent to transform the at least one cell with the heterologous polynucleotide molecule, wherein a force treatment is applied to the embryo explant in contact with the inoculation medium.

In another aspect, the present disclosure provides a method of producing a genetically modified monocot plant cell comprising: applying a force treatment to a monocot seed embryo explant comprising meristematic tissue, and transforming at least one plant cell of the embryo explant with a heterologous polynucleotide molecule by inoculating the embryo explant with an inoculation medium comprising a Rhizobiales bacterium competent to transform the at least one cell with the heterologous polynucleotide molecule.

In yet another aspect, the present disclosure provides a method of introducing a heterologous polynucleotide molecule into the monocot seed embryo explant by inoculation with a Rhizobiales bacterium comprising the heterologous polynucleotide molecule in an inoculation medium followed by co-culture of the monocot seed embryo explant in contact with a co-culture medium.

In still yet another aspect, the present disclosure provides a method of producing a genetically modified monocot plant or plant part comprising: introducing a heterologous polynucleotide molecule into at least one cell of a monocot seed embryo explant comprising meristematic tissue, culturing the embryo explant in contact with a first bud induction medium comprising a first auxin and a first cytokinin, and regenerating the genetically modified monocot plant or plant part from the cultured the embryo explant in contact with a regeneration medium, wherein the regeneration medium has a low salt concentration.

In a further aspect, the present disclosure provides a bud induction medium (or first bud induction medium) comprising a first auxin and a first cytokinin. An embryo explant, in one embodiment, is cultured in contact with the first bud induction medium for a time period in a range from about 2 days to about 14 days or from about 6 days to about 8 days. An embryo explant, in another embodiment, is cultured in contact with the first bud induction medium at a temperature in a range selected from the group consisting of about 20° C. to about 40° C., about 25° C. to about 30° C., about 30° C. to about 40° C., about 30° C. to about 37° C., and about 33° C. to about 35° C. In yet another embodiment, the first bud induction medium comprises a high cytokinin to auxin ratio.

Non-limiting examples of auxins that may be used in the first bud induction medium include 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba). The concentration of the first auxin in the first bud induction medium, in one embodiment, is from about 0.02 mg/L to about 25 mg/L or from about 1 mg/L to about 2 mg/L. In some embodiments, the concentration of the first auxin in the first bud induction medium is from about 0.1 mg/L to about 10 mg/L or about 0.5 mg/L to about 4 mg/L. In further embodiments, the first auxin is 2,4-D and the concentration of 2,4-D in the first bud induction medium is about 0.1 mg/L to about 10 mg/L, about 0.1 mg/L to about 4 mg/L, or about 1 mg/L; or the first auxin is picloram and the concentration of picloram in the first bud induction medium is about 2 mg/L.

Non-limiting examples of cytokinins that may be used in the first bud induction medium include 6-benzylaminopurine (BAP), thidiazuron (TDZ), kinetin, zeatin, diphenyl urea (DPU), 6-(gamma,gamma-dimethylallylamino)purine (2iP), and meta-topolin. The concentration of the first cytokinin in the first bud induction medium, in another embodiment, is in a range from about 0.1 mg/L to about 50 mg/L, about 0.5 mg/L to about 25 mg/L, about 1 mg/L to about 20 mg/L, about 5 mg/L to about 15 mg/L, or about 1 mg/L to about 5 mg/L. In yet another embodiment, the first cytokinin is BAP and the concentration of the BAP in the first bud induction medium is about 2 mg/L to about 25 mg/L, about 10 mg/L. In still yet another embodiment, the first cytokinin is TDZ and the concentration of the TDZ in the first bud induction medium is about 2 mg/L. In one embodiment, the first bud induction medium is a solid medium.

In further embodiments, culturing the monocot seed embryo explant with a first bud induction medium comprises culturing the explant with a photoperiod of about 16 hours light and about 8 hours dark. In still further embodiments, the photoperiod is about 16 hours light and about 8 hours dark for about 6 days to about 24 days. In some embodiments, the light intensity during the culturing is about 90 μMol m⁻² sec⁻¹ to about 160 μMol m⁻² sec⁻¹ at a wavelength of about 400-700 nm. In particular embodiments, the light intensity is about 90 μMol m⁻² sec⁻¹ at a wavelength of about 400-700 nm.

In particular embodiments, the second bud induction medium may comprise the first auxin and the first cytokinin; or the second bud induction medium may comprise the first auxin and the second cytokinin; or the second bud induction medium may comprise the second auxin and the first cytokinin; or the second bud induction medium may comprise the second auxin and the second cytokinin. In certain embodiments, the first auxin is selected from the group consisting of: 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba). In some embodiments, the first auxin is 2,4-dichlorophenoxy-acetic acid (2,4-D). In other embodiments, the first auxin is 4-amino-3,5,6-trichloro-picolinic acid (picloram). In other embodiments, the concentration of the first auxin or the second auxin in the second bud induction medium is from about 0.1 mg/L to about 10 mg/L or about 0.5 mg/L to about 4 mg/L.

In other embodiments, the first cytokinin is selected from the group consisting of: 6-benzylaminopurine (BAP), thidiazuron (TDZ), kinetin, zeatin, diphenyl urea (DPU), 6-(gamma,gamma-dimethylallylamino)purine (2iP), and meta-topolin. In certain embodiments, the first cytokinin is 6-benzylaminopurine (BAP). In certain embodiments, the first cytokinin is thidiazuron (TDZ). Further, in some embodiments, the second bud induction medium comprises the first cytokinin and the concentration of the first cytokinin in the second bud induction medium is from about 0.5 mg/L to about 50 mg/L, 0.5 mg/L to about 25 mg/L, about 1 mg/L to about 20 mg/L, about 5 mg/L to about 15 mg/L, or about 1 mg/L to about 5 mg/L. In particular embodiments, the first cytokinin is TDZ and the concentration of TDZ in the second bud induction medium is about 2 mg/L; or the first cytokinin is BAP and the concentration of BAP in the second bud induction medium is about 10 mg/L.

In some embodiments, the second bud induction medium comprises the second auxin and the second auxin is selected from the group consisting of: 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba). In certain embodiments, the second auxin is 4-amino-3,5,6-trichloro-picolinic acid (picloram). In other embodiments, the second auxin is 2,4-dichlorophenoxy-acetic acid (2,4-D). In yet other embodiments, the second bud induction medium comprises the second cytokinin and the second cytokinin is selected from the group consisting of: 6-benzylaminopurine (BAP), thidiazuron (TDZ), kinetin, zeatin, diphenyl urea (DPU), 6-(gamma,gamma-dimethylallylamino)purine (2iP), and meta-topolin. In certain embodiments, the second cytokinin is thidiazuron (TDZ). In particular embodiments, the second cytokinin is 6-benzylaminopurine (BAP).

In particular embodiments, the monocot seed embryo explant is cultured in contact with the second bud induction medium for a time period in a range from about 4 days to about 28 days. In certain embodiments, the monocot seed embryo explant is cultured in contact with the second bud induction medium for a time period in a range from about 7 days to about 14 days. In certain embodiments, the monocot seed embryo explant is cultured in contact with the second bud induction medium at a temperature in a range from about 20° C. to about 32° C. or about 25° C. to about 29° C. In certain embodiments, the monocot seed embryo explant is cultured in contact with the second bud induction medium at a temperature of about 27° C. or about 28° C.

In some embodiments, the second bud induction medium may comprise a high cytokinin to auxin ratio. In certain embodiments, the concentration of the first auxin or the second auxin in the second bud induction medium is from about 0.1 mg/L to about 10 mg/L or about 0.5 mg/L to about 4 mg/L. In particular embodiments, the second bud induction medium comprises the first auxin, and the concentration of the first auxin in the second bud induction medium is in a range from about 0.1 mg/L to about 10 mg/L or about 0.5 mg/L to about 4 mg/L, or is about 1 mg/L or about 2 mg/L. In certain embodiments, the first auxin is picloram and the concentration of picloram in the second bud induction medium is about 2 mg/L; or wherein the second auxin is 2,4-D and the concentration of 2,4-D in the second bud induction medium is about 1 mg/L. In some embodiments, the second bud induction medium comprises the second auxin, and the concentration of the second auxin in the second bud induction medium is in a range from about 0.1 mg/L to about 10 mg/L or about 0.5 mg/L to about 4 mg/L, or is about 1 mg/L or about 2 mg/L. In other embodiments, the second auxin is picloram and the concentration of picloram in the second bud induction medium is about 2 mg/L; or wherein the second auxin is 2,4-D and the concentration of 2,4-D in the second bud induction medium is about 1 mg/L. In some embodiments, the second bud induction medium comprises the second cytokinin and the concentration of the second cytokinin in the second bud induction medium is in a range from about 0.5 mg/L to about 50 mg/L, about 0.5 mg/L to about 25 mg/L, about 1 mg/L to about 20 mg/L, about 5 mg/L to about 15 mg/L, or about 1 mg/L to about 5 mg/L. In certain embodiments, the second cytokinin is TDZ and the concentration of TDZ in the second bud induction medium is about 2 mg/L; or the second cytokinin is BAP and the concentration of BAP in the second bud induction medium is about 10 mg/L.

In additional embodiments, the first bud induction medium comprises the first auxin and the second bud induction medium comprises the second auxin, and the first auxin is different than the second auxin. In these or further embodiments, the first bud induction medium comprises the first cytokinin and the second bud induction medium comprises the second cytokinin, and the first cytokinin is different than the second cytokinin. In certain embodiments, the first cytokinin in the first bud induction medium is 6-benzylaminopurine (BAP) and the second cytokinin in the second bud induction medium is thidiazuron (TDZ). In some embodiments, the first auxin in the first bud induction medium is 2,4-dichlorophenoxy-acetic acid (2,4-D) and the second auxin in the second bud induction medium is 4-amino-3,5,6-trichloro-picolinic acid (picloram). In certain embodiments, the second bud induction medium comprises the second auxin, and the second auxin is 4-amino-3,5,6-trichloro-picolinic acid (picloram), wherein the concentration of 4-amino-3,5,6-trichloro-picolinic acid (picloram) in the second bud induction medium is in a range from about 0.1 mg/L to about 10.0 mg/L, wherein the second cytokinin in the second bud induction medium is thidiazuron (TDZ), and wherein the concentration of thidiazuron (TDZ) in the second bud induction medium is in a range from about 0.5 mg/L to about 15 mg/L. In further particular embodiments, the concentration of picloram in the second bud induction medium is from about 0.5 mg/L to about 4 mg/L. In still further embodiments, the concentration of TDZ in the second bud induction medium is from about 1 mg/L to about 4 mg/L. In one embodiment, the second bud induction medium is a solid medium.

In further embodiments, the heterologous polynucleotide molecule comprises a selectable marker gene, wherein the second bud induction medium comprises a selection agent, and wherein the selectable marker gene provides resistance in a plant to the selection agent. In certain embodiments, the selection agent is selected from the group consisting of kanamycin, paromomycin, hygromycin B, spectinomycin, streptomycin, gentamycin, glyphosate, glufosinate, phosphinothricin, bromoxynil, bialaphos, dicamba, imidazolinone, and sulfonylurea.

In some embodiments, culturing the monocot seed embryo explant in a first bud induction medium and/or a second bud induction medium comprises culturing the explant with a photoperiod of about 16 hours light and about 8 hours dark. In certain embodiments, the monocot seed embryo explant is exposed to a light intensity during culturing of the monocot seed embryo explant in the first bud induction medium and/or the second bud induction medium of about 90 μMol m⁻² sec⁻¹ to about 160 μMol m⁻² sec⁻¹. In particular embodiments, the light intensity is about 90 μMol m⁻² sec⁻¹.

In yet another aspect, the present disclosure provides a regeneration medium. The genetically modified monocot plant or plant part, in one embodiment, is regenerated in contact with the regeneration medium at a temperature in a range from about 20° C. to about 32° C., from about 25° C. to about 29° C., or from about 27° C. to about 28° C. In another embodiment, the genetically modified monocot plant or plant part is regenerated in contact with the regeneration medium for a time period in a range from about 20 days to about 50 days or from about 28 days to about 42 days. The regeneration medium, in yet another embodiment, has a low salt concentration. In still yet another embodiment, the regeneration medium does not contain an auxin or a cytokinin. The regeneration medium, in one embodiment, does not contain a surfactant. In another embodiment, the regeneration medium comprises a selection agent as described herein. In yet another embodiment, the regeneration medium is a solid medium. In still yet another embodiment, the methods provided herein further comprise preparing a genetically modified plant part from the genetically modified plant.

The heterologous polynucleotide molecule may comprise a selectable marker gene, wherein the regeneration medium comprises a selection agent, and wherein the selectable marker gene provides resistance in a plant to the selection agent. In some embodiments, the selection agent is selected from the group consisting of kanamycin, paromomycin, hygromycin B, spectinomycin, streptomycin, gentamycin, glyphosate, glufosinate, phosphinothricin, bromoxynil, bialaphos, dicamba, imidazolinone, and sulfonylurea. In some embodiments, the genetically modified monocot plant or plant part is non-chimeric. The regeneration medium may also be a solid medium. In other embodiments, such media may be liquid media. The embryo explant and the genetically modified monocot plant or plant part, in another embodiment, are cultured and regenerated without producing a callus tissue culture. In yet another embodiment, the present disclosure provides a method of preparing a genetically modified plant part from the genetically modified plant.

In one embodiment, the total salt concentration in the regeneration medium is less than about 2800 mg/L, less than about 2500 mg/L, or in a range from about 2200 mg/L to about 2500 mg/L. In another embodiment, the regeneration medium is a solid medium. In yet another embodiment, the embryo explant and the genetically modified monocot plant or plant part are cultured and regenerated without producing a callus tissue culture. In still yet another embodiment, the present disclosure further provides a method preparing a genetically modified plant part from the genetically modified plant.

In some embodiments, the present disclosure provides a regeneration medium defined by its total nitrogen, nitrate ion, ammonium ion, potassium ion, or sulfate ion concentration. The total nitrogen concentration in the regeneration medium, in one embodiment, is about 0.5 mM to about 20 mM, about 5 mM to about 20 mM, about 5 mM to about 15 mM, about 5 mM to about 10 mM, about 10 mM to about 15 mM, about 10 mM to about 20 mM, or about 15 mM to about 20 mM. The nitrate ion concentration in the regeneration medium, in another embodiment, is about 0.5 mM to about 20 mM, about 5 mM to about 20 mM, about 5 mM to about 15 mM, about 5 mM to about 10 mM, about 10 mM to about 15 mM, about 10 mM to about 20 mM, or about 15 mM to about 20 mM. The ammonium ion concentration in the regeneration medium, in yet another embodiment, is about 0.5 mM to about 15 mM, about 2.5 mM to about 15 mM, about 2.5 mM to about 10 mM, about 2.5 mM to about 5 mM, about 5 mM to about 15 mM, about 5 mM to about 10 mM, or about 10 mM to about 15 mM. The potassium ion concentration in the regeneration medium, in still yet another embodiment, is about 0.5 mM to about 15 mM, about 2.5 mM to about 15 mM, about 2.5 mM to about 10 mM, about 2.5 mM to about 5 mM, about 5 mM to about 15 mM, about 5 mM to about 10 mM, or about 10 mM to about 15 mM. The sulfate ion concentration in the regeneration medium, in one embodiment, is greater than or equal to about 5 mM or is about 5 mM to about 20 mM, about 5 mM to about 15 mM, about 5 mM to about 10 mM, about 10 mM to about 15 mM, about 10 mM to about 20 mM, or about 15 mM to about 20 mM.

In certain embodiments, the present disclosure provides a regeneration medium defined by its ammonium nitrate, calcium chloride, calcium nitrate, or potassium sulfate concentration. The ammonium nitrate concentration in the regeneration medium, in one embodiment, is about 100 mg/L to about 1000 mg/L, about 100 mg/L to about 750 mg/L, about 100 mg/L to about 500 mg/L, about 100 mg/L to about 250 mg/L, or about 250 mg/L to about 500 mg/L. The calcium chloride concentration in the regeneration medium, in another embodiment, is less than or equal to about 100 mg/L or is about 50 mg/L to about 100 mg/L or about 50 mg/L to about 75 mg/L. The calcium nitrate concentration in the regeneration medium, in yet another embodiment, is less than or equal to about 500 mg/L or is about 100 mg/L to about 500 mg/L, about 100 mg/L to about 300 mg/L, about 300 mg/L to about 400 mg/L, or about 100 mg/L to about 200 mg/L. The potassium sulfate concentration in the regeneration medium, in still yet another embodiment, is greater than or equal to about 500 mg/L or is about 500 mg/L to about 750 mg/L, about 500 mg/L to about 1000 mg/L, about 500 mg/L to about 1500 mg/L, about 500 mg/L to about 2000 mg/L, about 750 mg/L to about 1000 mg/L, or about 1000 mg/L.

In another aspect, the present disclosure provides a heterologous polynucleotide molecule comprising a selectable marker gene, wherein the regeneration medium comprises a selection agent, and wherein the selectable marker gene provides resistance in a plant to the selection agent. Non-limiting examples of selection agent that may be used according to the present disclosure include kanamycin, paromomycin, hygromycin B, spectinomycin, streptomycin, gentamycin, glyphosate, glufosinate, phosphinothricin, bromoxynil, bialaphos, dicamba, imidazolinone, and sulfonylurea.

In some embodiments, the genetically modified monocot plant or plant part is a corn (Zea mays) plant or plant part, a wheat plant or plant part, a rice plant or plant part, a barley plant or plant part, a turfgrass plant or plant part, or a sorghum plant or part. In certain embodiments, the genetically modified monocot plant or plant part is a corn (Zea mays) plant or plant part. In certain other embodiments, the genetically modified monocot plant or plant part is a wheat plant or plant part. In some embodiments, a genetically modified monocot plant is regenerated from the cultured monocot seed embryo explant. In particular embodiments, the genetically modified monocot plant is a corn (Zea mays) plant, a wheat plant, a rice plant, a barley plant, a turfgrass plant, or a sorghum plant. Other embodiments may further comprise preparing the monocot seed embryo explant from a monocot plant seed. In particular embodiments, the monocot plant seed is a corn (Zea mays) plant seed, a wheat plant seed, a rice plant seed, a barley plant seed, a turfgrass plant seed, or a sorghum plant seed. The monocot seed embryo explant may further be prepared under conditions wherein the explant does not germinate and remains viable and competent for genetic transformation. In some embodiments, the monocot seed embryo explant has an internal moisture content in a range from about 3% to about 25% prior to introducing the heterologous polynucleotide molecule. In certain embodiments, the monocot seed from which the monocot seed embryo explant is prepared has an internal moisture content in a range from about 3% to about 25%. Further, the heterologous polynucleotide molecule may comprise a gene of interest providing a beneficial agricultural trait when expressed in a plant, and wherein the genetically modified monocot plant or plant part is stably transformed with the gene of interest. The heterologous polynucleotide molecule may comprise one or more expression cassettes encoding a guide RNA and/or site-directed nuclease, and wherein the genetically modified monocot plant or plant part is transformed with the one or more expression cassettes.

In one aspect, the present disclosure provides an embryo explant for use according to the compositions and methods described herein. The embryo explant is prepared, in one embodiment, from a monocot seed under conditions wherein the embryo explant does not germinate and remains viable and competent for genetic transformation. The monocot seed from which the embryo explant is prepared, in another embodiment, has an internal moisture content in a range from about 3% to about 25%. The embryo explant, in yet another embodiment, is a dry mature corn seed embryo explant. In still yet another embodiment, the embryo explant has an internal moisture content in a range from about 3% to about 25% prior to introducing the heterologous polynucleotide molecule. The embryo explant, in one embodiment, is comprised of the apical portion of the embryo axis lacking the radical, and the remaining portions of the corn seed have been substantially removed from the embryo explant. In certain embodiments, the disclosure further comprises preparing the monocot seed embryo explant from a monocot plant seed.

### BRIEF DESCRIPTION OF THE DRAWINGS

The following drawings form part of the present specification and are included to further demonstrate certain aspects of the present disclosure. The invention may be better understood by reference to one or more of these drawings in combination with the detailed description of specific embodiments presented herein.

FIG. 1 demonstrates transient gene expression of transformed corn explants exposed to a gravitational force either prior to or during Agrobacterium inoculation.

FIG. 2 demonstrates transient gene expression of transformed corn explants exposed to a gravitational force during Agrobacterium inoculation at various temperatures.

FIG. 3 demonstrates transient gene expression of transformed wheat explants exposed to various gravitational forces during Agrobacterium inoculation.

FIG. 4 shows images of the effect of different gravitational forces (291 g, 1164 g, 2619 g and 4657 g) during inoculation on transient GUS expression in two experiments.

FIG. 5 shows transient GUS expression six days after Agrobacterium inoculation and co-culture. FIG. 5, Panel A-1 shows GUS expression in explants inoculated with Agrobacterium at an OD of 0.25 and co-cultured without added Agrobacterium; FIG. 5, Panel B-1 shows GUS expression in explants inoculated with Agrobacterium at an OD of 0.25 and co-cultured with added 1.25 mL of Agrobacterium at OD 0.25; FIG. 5, Panel C-1 shows GUS expression in explants inoculated with Agrobacterium at an OD of 0.5 and co-cultured with added 1.25 mL of Agrobacterium at OD 0.5; and FIG. 5, Panel D-1 shows GUS expression in explants inoculated with Agrobacterium at an OD of 1.0 and co-cultured with added 1.25 mL of Agrobacterium at OD 1.0.

FIG. 6 shows Agrobacterium transformation of crushed Setaria seeds. FIG. 6, Panel A shows regenerated shoots 8 weeks after inoculation; FIG. 6, Panel B shows leaves from the regenerated shoots with GUS expression after 1 hour in X-gluc solution, confirming transformation; FIG. 6, Panel C shows a Southern hybridization of DNA isolated from the regenerated plants grown in the greenhouse (see example of plant in FIG. 6, Panel D) using a probe specific to the CP4 gene confirming that transgenic plants contained a single copy of the CP4 transgene.

FIG. 7 shows a seed rolling assembly (FIG. 7, Panel A) for removal of seed coat from Setaria seeds; FIG. 7, Panel B shows Setaria seeds before removal of seed coat; and FIG. 7, Panel C shows Setaria seeds after removal of seed coat.

FIG. 8 shows the progression of Agrobacterium-mediated transformation, culturing and regeneration of plants from intact Setaria seeds. FIG. 8, Panel A shows Setaria seeds after Agrobacterium infection; FIG. 8, Panel B shows GUS expression around the meristem region after X-gluc staining; FIG. 8, Panel C shows newly formed buds around the meristem region near leaf base where GUS staining was observed; and FIG. 8, Panel D shows regenerated plants following selection and rooting.

FIG. 9 shows GUS expression in reproductive tissues and in R1 seedlings of transgenic Setaria viridis plants. The top panels in FIG. 9 show GUS expression in anthers and pollen stigma (top left); spikelet (top middle); and immature seed (top right). The bottom panels in FIG. 9 show GUS expression in seedlings for three different transgenic events (Events 1, 3, 4).

### DETAILED DESCRIPTION OF THE INVENTION

The following is a detailed description provided to aid those skilled in the art in practicing the present inventions. Modifications and variations to the embodiments described herein can be made without departing from the spirit or scope of the present inventions. Compositions and methods are provided for improving the transformation of monocot seed excised embryo explants, which may include one or more steps of explant preparation, explant rehydration, Agrobacterium inoculation and co-culture, bud induction, extended bud induction, and/or regeneration of genetically modified plants or plant parts as described herein.

### A. Explant Preparation

In accordance with embodiments of the present disclosure, one or more monocot seed embryo explants can be produced from monocot plant seeds for production of genetically modified monocot plants or plant parts. Such plant seeds may be taken or harvested from plants grown in a field or greenhouse. Such monocot plant seeds can be a mature or immature monocot plant seeds but may preferably be mature monocot plant seeds. Examples of monocot plants include, but are not limited to, corn plants, wheat plants, rice plants, barley plants, rye plants, millet plants, oat plants, turfgrass plants, and sorghum plants. Examples of monocot plant seeds include, but are not limited to, corn or maize seeds, wheat seeds, rice seeds, barley seeds, rye seeds, millet seeds, oat seeds, turfgrass seeds, and sorghum seeds. Use of mature monocot plant seeds may provide the benefits or advantages of improved seed storage, explant preparation, and/or culturing. Examples of monocot plants or plant seeds that may be genetically modified or transformed according to present embodiments include any plant species within the Poaceae or Gramineae family of monocot or cereal plants and grasses, which may include any Zea genus corn or maize species, such as Zea mays, any Oryza genus rice species, such as Oryza sativa, any Triticum genus wheat species, such as Triticum aestivum or Triticum turgidum var durum, any Hordeum genus barley species, such as Hordeum vulgare, any Avena genus oat species, such as Avena sativa, any Sorghum genus sorghum species, such as Sorghum bicolor or Sorghum vulgare, any Secale genus rye species, such as Secale cereale, any Saccharum sugarcane species, or any Setaria, Pennisetum, Eleusine, Echinochloa, or Panicum genus millet species, such as Setaria virdis, Setaria italica, Pennisetum glaucum, Eleusine coracana, Echinochloa frumentacea, Panicum sumatrense, or Panicum miliaceum.

According to some embodiments, methods and compositions are provided for preparing, culturing, selecting and using explants, as well as the explants or cultured explants produced thereby. As used herein, the term “explant” or “seed embryo explant” refers to a plant part or plant tissue that is capable of being genetically modified and subsequently regenerated into a genetically modified plant or plant part. An “explant” or “seed embryo explant” may refer to a plant seed or any part of a plant seed, which may comprise at least a portion of a plant seed embryo. An “explant” or “seed embryo explant” may comprise an embryo explant excised from a plant seed that may comprise at least a part of an embryo meristem tissue. Alternatively, an “explant” or “seed embryo explant” may refer to a whole or intact plant seed, or a crushed, deformed or partially opened plant seed that may be produced by any suitable mechanical process. As used in reference to an explant or seed embryo explant, “partially opened” refers to an altered state of a plant seed that has one or more openings or fissures in the plant seed introduced by a mechanical force, such as squeezing, crushing, rolling, pressing, extruding, etc. An explant or seed embryo explant that is a whole or intact plant seed or a crushed, deformed or partially opened plant seed may in many cases have its seed coat removed. As used herein, a “genetically modified” plant, plant part, plant tissue, explant, or plant cell comprises a genetic modification or transgene introduced into the genome of the plant, plant part, plant tissue, explant, or plant cell through genetic engineering, which may be via a genetic transformation or a genome editing technique. As used herein, a “transgenic” plant, plant part, plant tissue, explant or plant cell has an exogenous nucleic acid sequence, polynucleotide, expression cassette, and/or transgene integrated into the genome of the plant, plant part, plant tissue, explant, or plant cell. In certain embodiments, explants according to the disclosure may be produced manually or using an automated process. For example, seed tissues may be removed from a seed by cutting, grinding, abrasion, or any other similar process. Manual or automated methods for removal of unnecessary seed parts may also be carried out. Fluid, for example, can be used to move explants and separate desirable explants from debris during mechanized handling of seeds, including compressed air, other gases, and liquids. Embryo explants may be excised from dry, dried, or wet seeds. Mature pant seeds may become drier as part of their normal maturation process, although seeds may be further dried prior to excision and/or explants may be dried after excision from seeds. Dry or dried excision of plant embryo explants may be performed for their immediate use or for later use after storage for a period of time. Explant preparation may further comprise drying the seed and/or explant to obtain a desired moisture content of the seed and/or explant for improved storage preparation (e.g., excision) or use, depending upon the initial moisture content of the seed or explant without drying. Following excision, the explant may be purified or isolated from other seed material and debris by rinsing, flotation, or other methods known in the art.

In other embodiments, a seed or explant prepared or used in accordance with the embodiments of the present disclosure may be defined as having an internal moisture of about 3% to about 25%, about 3% to about 20%, about 3% to about 15%, about 3% to about 10%, about 5% to about 10%, including about 3%, 4%, 5%, 6%, 7%, 8%, 9%, 10%, 11%, 12%, 13%, 14%, 15%, 16%, 17%, 18%, 19%, 20%, 21%, 22%, 23%, 24%, or about 25% internal moisture, including all ranges derivable therebetween. An explant may be produced from a mature seed having a moisture content as described herein. In specific aspects, the moisture content of the seed or explant may be measured prior to or after explant excision, explant storage, while an explant is in storage, prior to explant rehydration, and/or prior to genetic modification or transformation.

In one aspect, any monocot embryo explant may be prepared or used according to the embodiments of the present disclosure. In particular embodiments, the monocot embryo explant may be a mature embryo, an immature embryo, meristematic tissue, callus tissue, or any other tissue that is transformable and regenerable. In some embodiments, the mature embryo explant is a dry excised explant. Dry excised explants may be taken from seeds and used almost directly as targets for transformation or genetic modification. In one embodiment, dry excised explants may be taken from mature dry seeds and used as targets for transformation or genetic modification with perhaps only minimal wetting, hydration, or pre-culturing steps. In other embodiments, wet, dried wet, or wet excised embryo explants may be used as a target for transformation or genetic modification. As used herein “wet” embryo explants refer to dry excised explants subjected to wetting, hydration, imbibition, or other minimal culturing steps prior to transformation or genetic modification. As used herein “dried wet” embryo explants refer to embryo explants which are primed for germination by wetting and then dried to arrest germination. As used herein “wet excised” explants refer to explants excised from imbibed or hydrated seeds. A wet embryo explant is hydrated or imbibed after excision from a seed, whereas a wet excised embryo explant is excised from an already hydrated or imbibed seed. As used herein a “callus” refers to a proliferating mass of unorganized, undifferentiated and/or dedifferentiated plant cells or tissue.

Explants for use according to the present disclosure may be genetically modified at various times after isolation, excision or removal from the mature monocot seed. In one embodiment, explants may have been removed from seeds for less than a day, for example, from about 1 to about 24 hours, such as about 1, 2, 3, 5, 7, 8, 9, 10, 11, 12, 13, 14, 15, 16, 17, 18, 19, 20, 21, 22, 23 or 24 hours prior to use. In other embodiments, explants may be stored for longer periods, including days, weeks, months, or years prior to use. Methods and parameters for drying, storing, and germinating seed are known in the art (e.g., U.S. Pat. No. 8,362,317, specifically incorporated herein by reference in its entirety, Senaratna et al., 1983, Pl. Physiol. 72:620-624, 1983; Vertucci and Roos, 1990, Pl. Physiol. 90:1019-1023, 1990; Chai et al., 1998, Seed Science Research 8 (Supplement 1):23-28, 1998). Any conditions may be used as desired, including incubation or storage at temperatures, for example, of about −80° C. to about 60° C.

The disclosure may in certain aspects involve sterilization of seeds or explants. Sterilization can include contacting seed or explant material with various liquid or gases that serve to reduce or eliminate the presence of viable bacterial or fungal contaminants that could otherwise interfere with seed or embryo viability. Sterilization by application of liquid may also hydrate or partially hydrate the plant seeds, explants, embryos or tissues and serve the purpose of priming the seeds, explants, embryos or tissues. Methods for sterilization include, but are not limited to, the use of chlorine gas, ozone, solutions of bleach or alcohol, ultraviolet light, temperatures of −20° C. or lower, and exposure to a temperature higher than 40° C.

In one aspect of the present disclosure, explants may be rehydrated prior to transformation or genetic modification. Rehydration media or solutions are known in the art and may comprise, for example, water, basal salts, macronutrients, micronutrients, and/or vitamins. The rehydration medium will typically not contain any plant hormones, such as an auxin or cytokinin. In one embodiment, rehydrating monocot seed embryo explants may be carried out for a period of time in a range from about 30 minutes to about 24 hours prior to transformation or genetic modification or any length of time within such range, such as for about 1 hour, about 1.5 hours, about 2 hours, or about 2.5 hours, or less than or equal to about 3 hours or less than or equal to about 2.5 hours, or less than or equal to about 2 hours, or in a range of about 1 hour to about 3 hours, or in a range of about 1 hour to about 2.5 hours, or in a range of about 1.5 hours to about 2.5 hours. Rehydration of embryo explants prior to transformation or genetic modification may improve transformation or editing frequency or the recovery of transformed or edited plants, as compared to explants that were not rehydrated, by at least about 0.2-fold, 0.4-fold, 0.6-fold, 0.8 fold, 1-fold, 2-fold, 3-fold, 4-fold, 5-fold, 10-fold, 15-fold, 20-fold, 25-fold, 30-fold, 35-fold, or 40-fold, including all ranges derivable therebetween. In a particular embodiment, rehydrating monocot seed embryo explants for at least about 2 hours prior to transformation or genetic modification may improve transformation or editing frequency or the recovery of transformed or edited plants, as compared to explants rehydrated for about 1 hour, by at least about 0.2-fold, 0.4-fold, 0.6-fold, 0.8-fold, 1-fold, 2-fold, 3-fold, 4-fold, 5-fold, or 10-fold, including all ranges derivable therebetween.

Improved transformation or editing of plants may be measured, in some embodiments of the present disclosure, by genotyping, transient expression, shoot frequency, percentage of normal shoots, normal shoot frequency, normal plant frequency, percentage of rooted shoots at one or more different steps or plant pulls, and/or overall, transformation frequency, plugging frequency, frequency of low copy number plants, and/or low copy number frequency. The methods described herein may, for example, improve transformation or editing of plants by at least about 0.1-fold, 0.2-fold, 0.4-fold, 0.6-fold, 0.8 fold, 1-fold, 1.5-fold, 2-fold, 2.5-fold, 3-fold, 3.5-fold, 4-fold, 4.5-fold, 5-fold, 7.5-fold, 10-fold, 15-fold, 20-fold, 30-fold, or 40-fold, including all ranges derivable therebetween.

### B. Inoculation and Force-Assisted Transformation

Methods and compositions are provided herein for genetic transformation or modification of monocot seed derived explants or monocot embryo explants. These explants may be defined, in one aspect or embodiment, as comprising meristematic tissue or embryonic meristem tissue, which contains plant cells that can differentiate or develop to produce multiple plant structures including, but not limited to, stem, roots, leaves, germ line tissue, and seeds. Indeed, an embryo explant may be defined as comprising all or part of a seed embryo removed from other non-embryonic seed tissues and further comprising all or part of a meristematic tissue or embryonic meristem tissue. Embodiments of the present disclosure may include genetically transforming or modifying at least one cell of the explant by introducing a heterologous polynucleotide molecule into the at least one cell by any suitable method or technique known in the art, such as electroporation, microprojectile or particle bombardment, microinjection, PEG-mediated transformation, Rhizobiales- or Agrobacterium-mediated transformation, and other modes of direct DNA uptake. All or part of the heterologous polynucleotide may then be transformed or incorporated into the genome of the plant cell, expressed into one or more editing molecules or tools (such as a guide RNA and/or site-specific nuclease), and/or provide a template for editing or site-directed integration. According to many embodiments, the heterologous polynucleotide is introduced into the at least one explant cell via Rhizobiales- or Agrobacterium-mediated transformation. The introducing or inoculation step may be carried out under ambient lighting conditions and may include subjecting the embryo explant to any force treatment.

According to embodiments of the present disclosure, a force treatment is applied to the monocot seed embryo explant either prior to or during inoculation, or prior to and during inoculation, of the monocot seed embryo explant with a Rhizobiales or Agrobacterium bacterium comprising the heterologous polynucleotide molecule. According to some embodiments, the force treatment is applied during and/or after rehydration of the monocot seed embryo explant. According to many embodiments, the force treatment can be applied during the inoculation step while the monocot seed embryo explant is in contact with the inoculation medium. In one embodiment, explants “in contact with” a medium may be positioned completely or partially in or on a medium. Non-limiting examples of medium in which an explant may be in contact with include a liquid medium, a solid medium, and a substrate comprising a medium. The monocot seed embryo explant may be submerged in a volume of the inoculation medium when the force treatment is applied. Alternatively, the force treatment may be applied to the monocot seed embryo explant after an excess amount of the inoculation medium has been removed. The inoculation medium, for example, may be decanted, poured, or blotted from the explant prior to application of the force treatment. If the force treatment is applied during the inoculation step, then the inoculation medium may not be entirely absent from contacting the monocot seed embryo explant, even if an amount or volume of the inoculation medium is removed from the explant before the force treatment.

As used herein, the term “heterologous polynucleotide molecule” refers to a polynucleotide molecule that is not naturally present, or is not naturally present in the same form, structure, etc., in the cell being transformed, without human intervention. For example, a heterologous polynucleotide molecule may not naturally occur in the plant species being transformed or may be expressed in a manner or genomic context that differs from the natural expression pattern or genomic context found in the species being transformed, (e.g., overexpressed). In one embodiment, the heterologous polynucleotide molecule may be the combination of two or more polynucleotide molecules, wherein such a combination is not normally found in nature. The two polynucleotide molecules may, in certain embodiments, be derived from different species or may be derived from different genes, such as, different genes from the same species or the same genes from different species. In particular embodiments, a heterologous polynucleotide molecule may comprise two polynucleotide sequences that are not found juxtaposed or operably linked in any naturally occurring polynucleotide molecule. In one embodiment, the heterologous polynucleotide molecule may comprise a promoter or other regulatory sequence operably linked to a transcribable polynucleotide sequence, wherein the promoter and the transcribable polynucleotide sequence are not operably linked in any naturally occurring polynucleotide molecule. As used herein, the term “polynucleotide molecule” refers to a linear or circular single-stranded or double-stranded DNA or RNA polynucleotide molecule or sequence, which may be derived from any source. For example, a polynucleotide molecule may comprise a polynucleotide sequence in which one or more nucleic acid sequences have been linked together in a functionally operative manner. As used herein the term “nucleic acid sequence” refers to deoxyribonucleic acid (DNA) or ribonucleic acid (RNA) sequence.

According to some embodiments, the polynucleotide molecule or heterologous polynucleotide molecule may be a recombinant polynucleotide molecule. As used herein, the term “recombinant” when used in reference to a polynucleotide (DNA or RNA) molecule, protein, construct, vector, etc., refers to a polynucleotide or protein molecule or sequence that is not naturally present, or is not naturally present in the same form, structure, etc., without human intervention. In one embodiment, a recombinant polynucleotide (DNA or RNA) molecule, protein, construct, vector, etc. may comprise, for example, a combination of two or more polynucleotide or protein sequences that do not naturally occur together in the same manner, such as a polynucleotide molecule, protein, construct, etc., comprising at least two polynucleotide or protein sequences that are operably linked but heterologous with respect to each other. In another embodiment, a recombinant polynucleotide (DNA or RNA) molecule, protein, construct, vector, etc. may comprise, for example, any combination of two or more polynucleotide or protein sequences in the same molecule (e.g., a plasmid, construct, vector, chromosome, protein, etc.) where such a combination is man-made and not normally found in nature. As used in this definition, the phrase “not normally found in nature” means not found in nature without human intervention. A recombinant polynucleotide or protein molecule, construct, etc., may comprise polynucleotide or protein sequence(s) that is/are (i) separated from other polynucleotide or protein sequence(s) that exist in proximity to each other in nature, and/or (ii) adjacent to (or contiguous with) other polynucleotide or protein sequence(s) that are not naturally in proximity with each other. Such a recombinant polynucleotide molecule, protein, construct, etc., may also refer to a polynucleotide or protein molecule or sequence that has been genetically engineered and/or constructed outside of a cell. For example, a recombinant DNA molecule may comprise any engineered or man-made plasmid, vector, etc., and may include a linear or circular DNA molecule. Such plasmids, vectors, etc., may contain various maintenance elements including, for example, a prokaryotic origin of replication and selectable marker, as well as one or more transgenes or expression cassettes perhaps in addition to a plant selectable marker gene, etc.

The concentration of the Rhizobiales bacterium or Agrobacterium in the inoculation medium can be measured and/or defined in terms of optical density (OD). As shown in the Examples, higher concentrations of Agrobacterium in the inoculation and/or co-culture mediums can improve or increase shoot frequency and plugging frequency, and thus transformation frequency. The OD concentration of the Rhizobiales bacterium or Agrobacterium in the inoculation medium can be in a range from about 0.1 to about 2.0, from about 0.1 to about 1.0, from about 0.1 to about 0.75, from about 0.1 to about 0.5, from about 0.2 to about 2.0, from about 0.2 to about 1.0, from about 0.2 to about 0.5, from about 0.25 to about 2.0, from about 0.5 to about 2.0, from about 0.5 to about 1.5, from about 0.75 to about 1.5, or from about 0.75 to about 1.25, or about 0.1, 0.2, 0.25, 0.5.0.75, 1.0, 1.25, 1.5, or 2.0, including all ranges derivable therebetween.

To improve transformation or editing of plants, a variety of different force treatments may be used or applied to the monocot seed embryo explant before and/or during the inoculation step, such as a centrifugal force treatment, a gravitational force treatment, a vacuum treatment, a sonication treatment, a vortexing treatment, a shearing treatment, a mechanical force treatment, and/or a pressure treatment, or any combination thereof. According to some embodiments, a force treatment may comprise a pressure treatment and/or a gravitational (or centrifugal) force treatment. According to some embodiments, a force treatment may comprise a pressure treatment. According to some embodiments, a force treatment may comprise a gravitational (or centrifugal) force treatment. In specific embodiments, the methods described herein may further comprise applying a mechanical force treatment, a vortexing treatment, a shaking or shearing treatment, a sonication treatment, and/or a vacuum treatment, in addition to a pressure treatment and/or a gravitational (or centrifugal) force treatment. Without being bound by theory, application of a force treatment prior to or during inoculation may improve transformation by increasing the contact and attachment of the Rhizobiales bacterium to the monocot seed embryo explant, by wounding the monocot seed embryo explant and/or by increasing the permeation of the Rhizobiales bacterium into meristematic or other explant tissues.

In some embodiments, the force treatment may comprise applying a pressure force or treatment in a range from about 100 pounds per square inch (psi) to about 20,000 psi, about 100 psi to about 18,000 psi, about 100 psi to about 16,000 psi, about 100 to about 14,000 psi, about 100 to about 12,000 psi, about 100 to about 10,000 psi, about 100 to about 8,000 psi, about 100 to about 6,000 psi, about 100 to about 4,000 psi, about 100 to about 2,000 psi, about 100 to about 1,000 psi, or about 100 psi to about 500 psi, such as about 100 psi, about 150 psi, about 200 psi, about 250 psi, about 300 psi, about 350 psi, about 400 psi, or about 500 psi, of pressure to the monocot seed embryo explant, including all ranges derivable therebetween. Other units for pressure are also known in the art. Methods for converting pressure in psi to other units, for example, standard atmospheres (atm) and Newtons (N) per square meter (N/m²) are known in the art. Pressure in atm can be accurately calculated using the following formula: atm=pressure (psi)/14.6959488, and 1 psi equals about 6894.76 N/m². Therefore, 100 psi is equal to about 6.80 atm, and 20,000 psi is equal to about 1360.9 atm. The pressure treatment can also be converted to an amount of force when the surface area is known or fixed. For example, the surface area of piston/cell cavity of the French Press 40K pressure cell (Thermo® IEC, FA-032) used in the Examples herein is about 0.88 in². Therefore, 3,334 psi applied using the French Press 40K pressure cell is equal to about 13,000 N [(3,334 psi×0.88 in²)]/[0.225 pounds/N] The pressure treatment, in some embodiments, may be applied from about 10 seconds to about 10 minutes, from about 15 seconds to about 8 minutes, from about 30 seconds to about 6 minutes, from about 2 minutes to about 4 minutes, or for about 3 minutes, including all ranges derivable therebetween.

The methods described herein comprise applying a gravitational or centrifugal force in a range from about 100×g to about 10,000×g, about 100×g to about 5,000×g, about 250×g to about 5,000×g, about 500×g to about 5,000×g, about 500×g to about 3,000×g, about 600×g to about 2,700×g, such as about 500×g, about 550×g, about 600×g, about 650×g, about 700×g, about 750×g, about 800×g, about 850×g, about 900×g, about 950×g, about 1000×g, about 1500×g, about 2000×g, about 2500×g, about 3000×g, about 3500×g, or about 4000×g, may be applied to the monocot seed embryo explant, including all ranges derivable therebetween. A non-limiting example of a gravitation force treatment which may be applied to the monocot seed embryo explant includes a centrifugal force or relative centrifugal force, which may be applied using an appropriate centrifuge. Methods for converting gravitational or centrifugal force, such as the relative centrifugal force (RCF) created by a centrifuge, to other units, such as revolutions per minute (rpm) and newton (N), are known in the art. Relative centrifugal force can be calculated based on the rpm and known dimensions of the device using the following formula: rpm=√[RCF/(r×1.118)×1×10⁵], wherein r=the rotational radius in centimeters. For the Sorvall™ RC3BP centrifuge (Thermo Fisher Scientific, Waltham, MA, USA) used in the Examples described herein, the rotational radius is about 24.67 cm. Therefore, 2620×g is equal to about 3,082 rpm [√[2620/(24.67×1.118)]×1×10⁵]. Similarly, centrifugal force in Newtons can be accurately estimated using the following formula: Force (N)=RCF×mass of the contents of the centrifugation tube (kg)×9.82 m/s². In one embodiment, if the mass of contents of the centrifugation tube may be about 0.05 kg, then 2620×g would be equal to about 1286 N [(2620×g)×0.05 kg×9.82 m/s²]. The gravitational or centrifugal force treatment may be applied, in some embodiments, in a range from about 1 minute to about 2 hours, about 2 minutes to about 110 minutes, about 5 minutes to about 90 minutes, about 10 minutes to about 90 minutes, about 10 minutes to about 80 minutes, about 10 minutes to about 70 minutes, about 10 minutes to about 60 minutes, about 10 minutes to about 50 minutes, about 15 minutes to about 45 minutes, or about 20 minutes to about 40 minutes, such as about 10 minutes, about 15 minutes, about 20 minutes, about 25 minutes, about 30 minutes about 35 minutes, about 40 minutes, about 45 minutes, about 50 minutes, about 55 minutes, or about 60 minutes (1 hour), including all ranges derivable therebetween.

The force treatment, such as the gravitational (or centrifugal) and/or pressure treatment(s), may be applied at a temperature of about 0.5° C. to about 28° C., about 2° C. to about 28° C., about 4° C. to about 28° C., about 10° C. to about 28° C., about 10° C. to about 25° C., or about 15° C. to about 23° C., including all ranges derivable therebetween.

The application of a force treatment as described herein may, in some embodiments, improve transformation or editing of plants by at least about 0.2-fold, 0.4-fold, 0.6-fold, 0.8 fold, 1-fold, 1.5-fold, 2-fold, 2.5-fold, 3-fold, 3.5-fold, 4-fold, 4.5-fold, or 5-fold, including all ranges derivable therebetween.

In one aspect of the methods provided herein, the force treatment may comprise applying both a pressure treatment and a gravitational force treatment to the monocot seed embryo explant. The pressure treatment and/or the gravitational force treatment may be applied prior to, during, or prior to and during inoculation of a monocot seed embryo explant with a bacterium from the order Rhizobiales, wherein the Rhizobiales bacterium comprises a heterologous polynucleotide for transforming, editing or genetically modifying at least one plant cell of the monocot seed embryo explant. In some embodiments, the pressure treatment is applied prior to applying the gravitational force treatment. In other embodiments, the gravitational force treatment is applied prior to the pressure treatment. The order of application of a pressure treatment and a gravitational force treatment may be preferred based on improved transformation or editing efficiency or frequency or based on ease of handling. In some embodiments, when a combination of pressure and gravitational force treatments are applied to monocot seed embryo explants, the pressure treatment may be applied before the gravitational force treatment, which may be due at least in part to the ability to apply the force treatment more evenly prior to pelleting the explants with the gravitational or centrifugal treatment. However, the centrifuged or pelleted explants could potentially be resuspended prior to a subsequent pressure treatment, or the pressure treatment could be applied to the centrifuged or pelleted explants without resuspension. In an aspect of the present disclosure, applying a pressure treatment and a gravitational force treatment either prior to, during, or prior to and during inoculation may improve transformation or editing of plants by at least about 0.2-fold, 0.4-fold, 0.6-fold, 0.8 fold, 1-fold, 1.5-fold, 2-fold, 2.5-fold, 3-fold, 3.5-fold, 4-fold, 4.5-fold, or 5-fold, including all ranges derivable therebetween, as compared to applying only the pressure treatment or only the gravitational force treatment.

In another aspect, the methods described herein may further comprise applying a vacuum treatment to the monocot seed embryo explant. The vacuum treatment may comprise, for example, submerging the monocot seed embryo explant in a liquid inoculation medium comprising a Rhizobiales bacterium and subjecting the monocot seed embryo explant to decreased pressure followed by rapid or gradual repressurization. Alternatively, a vacuum treatment may be applied to a monocot seed embryo explant that is not submerged in a liquid inoculation medium. The vacuum treatment, in some embodiments, may be applied before the force treatment is applied, after the force treatment is applied, before the gravitational force treatment is applied, after the gravitational force treatment is applied, before the pressure treatment is applied, and/or after the pressure treatment is applied. In particular embodiments, where the force treatment comprises applying a pressure treatment and a gravitational force treatment, a vacuum treatment may be applied between applying the pressure treatment and applying the gravitational force treatment, regardless as to whether the gravitational force treatment or the pressure treatment is applied first. In one embodiment, the monocot seed embryo explant may be subjected to a vacuum treatment of about 0.05 atm to about 0.50 atm, about 0.05 atm to about 0.40 atm, about 0.05 atm to about 0.30 atm, about 0.05 atm to about 0.20 atm, about 0.05 atm to about 0.10 atm, about 0.10 atm to about 0.50 atm, about 0.10 atm to about 0.40 atm, about 0.10 to about 0.30 atm of pressure, or about 0.10 atm to about 0.20 atm of pressure, including all ranges derivable therebetween.

### C. Co-Culture of Embryo Explants

Following inoculation of a monocot seed embryo explant(s) with a Rhizobiales or Agrobacterium comprising a heterologous polynucleotide to introduce the heterologous polynucleotide into at least one cell of the monocot seed embryo explant(s), the monocot seed embryo explant(s) may be co-cultured in contact with a co-culture medium. The co-culture medium may comprise, for example, water, basal salts, macronutrients, micronutrients, and/or vitamins. However, according to present embodiments, the co-culture medium may not contain any plant hormones, such as an auxin or cytokinin and/or any surfactant or wetting agent, although a plant hormone or auxin and/or a surfactant or wetting agent may alternatively be present in the co-culture medium. In certain embodiments, a surfactant may include any surfactant or combination of surfactants known in the art, for example a detergent, a wetting agent, an emulsifier, a foaming agent, or a dispersant. In one embodiment, the surfactant maybe be Silwet® or a similar surfactant. According to some embodiments, the monocot seed embryo explant(s) may be in contact with the co-culture medium at a temperature in a range from about 15° C. to about 25° C., or from about 17° C. to about 23° C., or from about 18° C. to about 20° C., or at a temperature of about 15° C., about 16° C., about 17° C., about 18° C., about 19° C., about 20° C., about 21° C., about 22° C., about 23° C., about 24° C., or about 25° C. According to some embodiments, the monocot seed embryo explant(s) may be in contact with the co-culture medium for a time period ranging from about 1 day to about 10 days, or from about 2 days to about 10 days, or from about 2 days to about 8 days, or from about 3 days to about 8 days, or from about 4 days to about 8 days, or from about 5 days to about 7 days, such as for about 2 days, about 3 days, about 4 days, about 5 days, about 6 days, about 7 days, or about 8 days. According to many embodiments, the monocot seed embryo explant(s) may be in contact with the co-culture medium for at least 5 days or at least 6 days. According to present embodiments, the co-culture medium in contact with the monocot seed embryo explant(s) may be a solid, liquid or semi-solid medium. According to present embodiments, the monocot seed embryo explant(s) may be in contact with a matrix, paper or mesh material or substrate, such as a Whatman or other filter paper, that is wetted, filled or soaked with a liquid co-culture medium. According to present embodiments, the monocot seed embryo explant(s) may be in contact with, but not submerged in, the co-culture medium.

The co-culturing step may also be carried out under a variety of lighting conditions. While some degree of lighting may generally be used, all or part of the co-culture step may alternatively be performed in the dark. The lighting treatments may be quantified in terms of the light/dark cycle and intensity of light, which may be expressed as the Photosynthetic Photon Flux Density (PPFD) in units of μ/m²·s. According to some embodiments, the co-culturing step may be carried out with an average or set light intensity of Photosynthetic Active Radiation (PAR) in a range from about 0 μ/m²·s to about 200 μ/m²·s, 20 μ/m²·s to about 200 μ/m²·s, 20 μ/m²·s to about 180 μ/m²·s, 30 μ/m²·s to about 180 μ/m²·s, 30 μ/m²·s to about 150 μ/m²·s, 30 μ/m²·s to about 120 μ/m² s, 60 μ/m²·s to about 120 μ/m²·s, 70 μ/m²·s to about 110 μ/m²·s, or 80 μ/m²·s to about 100 μ/m²·s. According to some embodiments, the co-culturing step may be carried out with an average or set light intensity of Photosynthetic Active Radiation (PAR) at about 0 μ/m²·s, about 10 μ/m²·s, about 20 μ/m²·s, about 30 μ/m²·s, about 40 μ/m²·s, about 50 μ/m²·s, about 60 μ/m²·s, about 70 μ/m²·s, about 80 μ/m²·s, about 90 μ/m²·s, about 100 μ/m²·s, about 110 μ/m²·s, about 120 μ/m²·s, about 130 μ/m²·s, about 140 μ/m²·s, about 150 μ/m²·s, about 160 μ/m²·s, about 170 μ/m²·s, about 180 μ/m²·s, about 190 μ/m²·s, or about 200 μ/m²·s. According to some embodiments, different amounts of light and dark cycles may be used during the co-culture step, which may comprise a presence of lighting for a length of time between about 0 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length.

According to some embodiments, the amounts of light and dark cycles during the co-culture step may be about 0 hours of light and about 24 hours of dark, about 1 hour of light and about 23 hours of dark, about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

According to some embodiments, the co-culture medium may comprise the Rhizobiales bacterium or Agrobacterium competent to transform at least one cell of the explant with the heterologous polynucleotide molecule. As shown in the Examples, higher concentrations of Agrobacterium in the inoculation and/or co-culture mediums can improve or increase shoot frequency and plugging frequency, and thus transformation frequency. The OD concentration of the Rhizobiales bacterium or Agrobacterium in the co-culture medium can be in a range from about 0.1 to about 2.0, from about 0.1 to about 1.0, from about 0.1 to about 0.75, from about 0.1 to about 0.5, from about 0.2 to about 2.0, from about 0.2 to about 1.0, from about 0.2 to about 0.5, from about 0.25 to about 2.0, from about 0.5 to about 2.0, from about 0.5 to about 1.5, from about 0.75 to about 1.5, or from about 0.75 to about 1.25, or about 0.1, 0.2, 0.25, 0.5. 0.75, 1.0, 1.25, 1.5, or 2.0, including all ranges derivable therebetween.

### D. Bud Induction and Extended Bud Induction

According to present embodiments, a monocot seed embryo explant that has been transformed or edited by introducing a heterologous polynucleotide molecule into at least one cell of the embryo explant may be cultured in contact with at least a first bud induction medium comprising an auxin and a cytokinin. The monocot seed embryo explant may have been inoculated with an inoculation medium comprising a Rhizobiales or Agrobacterium that comprises the heterologous polynucleotide molecule, and the monocot seed embryo explant may also have been co-cultured in contact with a co-culture medium, prior to the bud induction step.

As provided herein, the monocot seed embryo explant may be further cultured in contact with a second or extended bud induction medium comprising an auxin and a cytokinin and then cultured in contact with a regeneration medium to produce a genetically modified plant or plant part. In some embodiments, the methods described herein comprise culturing the monocot seed embryo explant in contact with a second bud induction medium after the monocot seed embryo explant is cultured in contact with the bud induction medium (or first bud induction medium) and before regenerating the genetically modified monocot plant or plant part from the cultured monocot seed embryo explant in contact with a regeneration medium. In another embodiment, the (first) bud induction medium and/or the second (or extended) bud induction medium may each comprise a high cytokinin to auxin ratio.

According to present embodiments, the bud induction medium (or first bud induction medium) and the second bud induction medium (or extended bud induction medium) may each comprise a variety of standard culture media or solution ingredients or components, such as for example, basal salts, macronutrients, micronutrients, sugars, antibiotics and/or vitamins. The bud induction medium (or first bud induction medium) and the second bud induction medium (or extended bud induction medium) may each comprise an auxin and a cytokinin. The bud induction medium (or first bud induction medium) and the second bud induction medium (or extended bud induction medium) may each comprise one or more selection agent(s), although according to many embodiments, a selection agent is absent in the first bud induction medium. The absence of the selection agent in the first bud induction medium may allow the first bud induction medium to function as a delay medium. The identity of the selection agent will typically depend on the selectable marker gene present in the heterologous polynucleotide molecule introduced into the monocot seed embryo explant. The bud induction medium (or first bud induction medium) and/or the second bud induction medium (or extended bud induction medium) may each be a solid, semi-solid or liquid medium, although each of these media may typically be a solid medium. A solid medium may comprise a gelling or polymeric agent or ingredient, such as agarose, etc., that can solidify and form the solid medium.

As used herein a “high cytokinin to auxin ratio” generally refers to a condition wherein the level of cytokinin activity is relatively high in comparison to the level of auxin activity present in the medium, which may typically be a cytokinin:auxin ratio of at least about 1:1 or higher in term of weight/vol; provided, however, that the exact cytokinin:auxin ratio will depend on the exact chemical identities of the auxin and cytokinin since different auxins and cytokinins can have different activities and/or modes of action as known in the art. The levels of cytokinin and auxin in a medium having a high cytokinin to auxin ratio may be present in the medium (measured in terms of weight/volume), for example, at a ratio of about 1:1, 1.5:1, 2:1, 2.5:1, 3:1, 3.5:1, 4:1, 4.5:1, 5:1, 5.5:1, 6:1, 6.5:1, 7:1, 7.5:1, 8:1, 8.5:1, 9:1, 9.5:1, 10:1, 10.5:1, 11:1, 11.5:1, 12:1, 12.5:1, 13:1, 13.5:1, 14:1, 14.5:1, or 15:1, including all ranges derivable therebetween.

The levels of cytokinin and auxin in a culture medium having a high cytokinin to auxin ratio may be, for example, greater than or equal to about 1:1 or at least about 1:1 or higher, greater than or equal to about 1.5:1 or at least about 1.5:1 or higher, greater than or equal to about 2:1 or at least about 2:1 or higher, greater than or equal to about 2.5:1 or at least about 2.5:1 or higher, greater than or equal to about 3:1 or at least about 3:1 or higher, greater than or equal to about 3.5:1 or at least about 3.5:1 or higher, greater than or equal to about 4:1 or at least about 4:1 or higher, greater than or equal to about 4.5:1 or at least about 4.5:1 or higher, greater than or equal to about 5:1 or at least about 5:1 or higher, greater than or equal to about 5.5:1 or at least about 5.5:1 or higher, greater than or equal to about 6:1 or at least about 6:1 or higher, greater than or equal to about 6.5:1 or at least about 6.5:1 or higher, greater than or equal to about 7:1 or at least about 7:1 or higher, greater than or equal to about 7.5:1 or at least about 7.5:1 or higher, greater than or equal to about 8:1 or at least about 8:1 or higher, greater than or equal to about 8.5:1 or at least about 8.5:1 or higher, greater than or equal to about 9:1 or at least about 9:1 or higher, greater than or equal to about 9.5:1 or at least about 9.5:1 or higher, greater than or equal to about 10:1 or at least about 10:1 or higher, greater than or equal to about 10.5:1 or at least about 10.5:1 or higher, greater than or equal to about 11:1 or at least about 11:1 or higher, greater than or equal to about 11.5:1 or at least about 11.5:1 or higher, or greater than or equal to about 12:1 or at least about 12:1 or higher, including all ranges derivable therebetween.

The levels of cytokinin and auxin in a culture medium having a high cytokinin to auxin ratio may be, for example, in a range between about 1:1 and about 12:1, about 2:1 and about 12:1, about 4:1 and about 12:1, about 6:1 and about 12:1, about 8:1 and about 12:1, about 1:1 and about 10:1, about 2:1 and about 10:1, about 4:1 and about 10:1, about 6:1 and about 10:1, about 8:1 and about 10:1, about 1:1 and about 8:1, about 2:1 and about 8:1, about 4:1 and about 8:1, about 6:1 and about 8:1, about 1:1 and about 6:1, about 2:1 and about 6:1, about 4:1 and about 6:1, about 1:1 and about 5:1, about 2:1 and about 5:1, about 3:1 and about 5:1, about 1:1 and about 4:1, about 2:1 and about 4:1, about 3:1 and about 4:1, about 1:1 and about 3:1, or about 1:1 and about 2:1, including all ranges derivable therebetween.

Non-limiting examples of cytokinins that may be used in the accordance with the present disclosure may include, but are not limited to: 6-benzylaminopurine (BAP), thidiazuron (TDZ), N-(2-chloro-4-pyridyl)-N-phenylurea (4-CPPU), kinetin, zeatin, diphenyl urea (DPU), 6-(gamma,gamma-dimethylallylamino)purine (2iP), and 6-(3-hydroxybenzylamino)purine (meta-topolin). Auxins which may be used in accordance with the present disclosure may include, but are not limited to: 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba).

According to present embodiments, the bud induction medium (or first bud induction medium) may comprise the same or different auxin and/or the same or different cytokinin than the second bud induction medium (or extended bud induction medium). The bud induction medium (or first bud induction medium) may comprise a first auxin and a first cytokinin, and the second bud induction medium or extended bud induction medium may comprise the first auxin or a second auxin and the first cytokinin or a second cytokinin. According to some embodiments, the second bud induction medium (or extended bud induction medium) may comprise the same auxin or a different auxin as the bud induction medium (or the first bud induction medium). According to some embodiments, the second bud induction (or extended bud induction medium) may comprise the same cytokinin or a different cytokinin as the bud induction medium (or first bud induction medium).

According to present embodiments, the concentration of the cytokinin (or two or more cytokinins) in the first bud induction medium and/or the second (or extended) bud induction medium is in a range from about 0.1 mg/L to about 100.0 mg/L, 1 mg/L to about 90.0 mg/L, 1 mg/L to about 80.0 mg/L, 1 mg/L to about 75.0 mg/L, 2 mg/L to about 90.0 mg/L, 2 mg/L to about 80.0 mg/L, 2 mg/L to about 75.0 mg/L, 5 mg/L to about 90.0 mg/L, 5 mg/L to about 80.0 mg/L, 5 mg/L to about 75.0 mg/L, 5 mg/L to about 70.0 mg/L, 10 mg/L to about 90.0 mg/L, 10 mg/L to about 80.0 mg/L, 10 mg/L to about 75.0 mg/L, 10 mg/L to about 70.0 mg/L, 15 mg/L to about 90.0 mg/L, 15 mg/L to about 80.0 mg/L, 15 mg/L to about 75.0 mg/L, 15 mg/L to about 70.0 mg/L, 20 mg/L to about 90.0 mg/L, 20 mg/L to about 80.0 mg/L, 20 mg/L to about 75.0 mg/L, 20 mg/L to about 70.0 mg/L, 20 mg/L to about 60.0 mg/L, 30 mg/L to about 90.0 mg/L, 30 mg/L to about 80.0 mg/L, 30 mg/L to about 75.0 mg/L, 30 mg/L to about 70.0 mg/L, 30 mg/L to about 60.0 mg/L, 40 mg/L to about 90.0 mg/L, 40 mg/L to about 80.0 mg/L, 40 mg/L to about 75.0 mg/L, 40 mg/L to about 70.0 mg/L, 40 mg/L to about 60.0 mg/L, about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 0.1 mg/L to about 15.0 mg/L, about 0.1 mg/L to about 12.5 mg/L, about 0.1 mg/L to about 10.0 mg/L, about 0.1 mg/L to about 7.5 mg/L, about 0.1 mg/L to about 5.0 mg/L, about 0.1 mg/L to about 4.0 mg/L, about 0.1 mg/L to about 3.0 mg/L, about 0.2 mg/L to about 15.0 mg/L, about 0.2 mg/L to about 12.5 mg/L, about 0.2 mg/L to about 10.0 mg/L, about 0.2 mg/L to about 7.5 mg/L, about 0.2 mg/L to about 5.0 mg/L, about 0.2 mg/L to about 4.0 mg/L, about 0.2 mg/L to about 3.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 5.0 mg/L, about 0.5 mg/L to about 4.0 mg/L, about 0.5 mg/L to about 3.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 5.0 mg/L, about 1.0 mg/L to about 5.0 mg/L, about 1.0 mg/L to about 4.0 mg/L, or about 1.0 mg/L to about 3.0 mg/L, including all ranges derivable therebetween. In some embodiments, the concentration of the cytokinin in the first bud induction medium or the second bud induction medium may be, for example, about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, about 25.0 mg/L, about 30 mg/L, about 40 mg/L, about 50 mg/L, about 60 mg/L, about 70 mg/L, about 75 mg/L, about 80 mg/L, about 90 mg/L, or about 100 mg/L, including all ranges derivable therebetween. The cytokinin in the first and second bud induction media may be the same or different, and each of these bud induction media may comprise one or more cytokinins.

In some embodiments, the concentration of the auxin (or two or more auxins) the first bud induction medium and/or the second (or extended) bud induction medium is in the range from about 0.01 mg/L to about 25.0 mg/L, about 0.05 mg/L to about 25 mg/L, about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, about 9.0 mg/L to about 11.0 mg/L, about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 10.0 mg/L, about 4.0 mg/L to about 7.5 mg/L, about 4.0 mg/L to about 7.0 mg/L, about 4.0 mg/L to about 6.0 mg/L, about 0.1 mg/L to about 15.0 mg/L, about 0.1 mg/L to about 12.5 mg/L, about 0.1 mg/L to about 10.0 mg/L, about 0.1 mg/L to about 7.5 mg/L, about 0.1 mg/L to about 5.0 mg/L, about 0.1 mg/L to about 4.0 mg/L, about 0.1 mg/L to about 3.0 mg/L, about 0.2 mg/L to about 15.0 mg/L, about 0.2 mg/L to about 12.5 mg/L, about 0.2 mg/L to about 10.0 mg/L, about 0.2 mg/L to about 7.5 mg/L, about 0.2 mg/L to about 5.0 mg/L, about 0.2 mg/L to about 4.0 mg/L, about 0.2 mg/L to about 3.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 5.0 mg/L, about 0.5 mg/L to about 4.0 mg/L, about 0.5 mg/L to about 3.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, about 1.5 mg/L to about 2.5 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, about 0.1 mg/L to about 1.0 mg/L, about 0.1 mg/L to about 0.75 mg/L, about 0.1 mg/L to about 0.7 mg/L, about 0.1 mg/L to about 0.6 mg/L, about 0.2 mg/L to about 1.0 mg/L, about 0.2 mg/L to about 0.75 mg/L, about 0.2 mg/L to about 0.7 mg/L, about 0.2 mg/L to about 0.6 mg/L, about 0.3 mg/L to about 1.0 mg/L, about 0.3 mg/L to about 0.75 mg/L, about 0.3 mg/L to about 0.7 mg/L, about 0.3 mg/L to about 0.6 mg/L, about 0.4 mg/L to about 1.0 mg/L, about 0.4 mg/L to about 0.75 mg/L, about 0.4 mg/L to about 0.7 mg/L, about 0.4 mg/L to about 0.6 mg/L, about 0.05 mg/L to about 7.5 mg/L, about 0.02 mg/L to about 5 mg/L, or about 0.75 mg/L to about 2.5 mg/L, including all ranges derivable therebetween. In some embodiments, the concentration of the auxin in the first bud induction or second bud induction medium may be, for example, about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable therebetween. The auxins in the first and second bud induction media may be the same or different, and each of these media may comprise one or more auxins.

In some embodiments, the first bud induction medium and/or the second (or extended) bud induction medium may comprise a cytokinin, wherein the cytokinin is 6-benzylaminopurine (BAP). In some embodiments, the concentration of BAP in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of BAP in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise a cytokinin, wherein the cytokinin is thidiazuron (TDZ). In some embodiments, the concentration of TDZ in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. In some embodiments, the concentration of TDZ in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise a cytokinin, wherein the cytokinin is N-(2-chloro-4-pyridyl)-N-phenylurea (4-CPPU). In some embodiments, the concentration of 4-CPPU in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. In some embodiments, the concentration of 4-CPPU in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise a cytokinin, wherein the cytokinin is kinetin. In some embodiments, the concentration of kinetin in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of kinetin in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise a cytokinin, wherein the cytokinin is zeatin. In some embodiments, the concentration of zeatin in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of zeatin in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise a cytokinin, wherein the cytokinin is 6-(gamma, gamma-dimethylallylamino)purine (2iP). In some embodiments, the concentration of 2iP in the first bud induction medium and/or the second (or extended) bud induction medium may be in the range from 5 mg/L to about 100.0 mg/L, 5 mg/L to about 90.0 mg/L, 5 mg/L to about 80.0 mg/L, 5 mg/L to about 75.0 mg/L, 5 mg/L to about 70.0 mg/L, 10 mg/L to about 100.0 mg/L, 10 mg/L to about 90.0 mg/L, 10 mg/L to about 80.0 mg/L, 10 mg/L to about 75.0 mg/L, 10 mg/L to about 70.0 mg/L, 15 mg/L to about 100.0 mg/L, 15 mg/L to about 90.0 mg/L, 15 mg/L to about 80.0 mg/L, 15 mg/L to about 75.0 mg/L, 15 mg/L to about 70.0 mg/L, 20 mg/L to about 100.0 mg/L, 20 mg/L to about 90.0 mg/L, 20 mg/L to about 80.0 mg/L, 20 mg/L to about 75.0 mg/L, 20 mg/L to about 70.0 mg/L, 20 mg/L to about 60.0 mg/L, 30 mg/L to about 100.0 mg/L, 30 mg/L to about 90.0 mg/L, 30 mg/L to about 80.0 mg/L, 30 mg/L to about 75.0 mg/L, 30 mg/L to about 70.0 mg/L, 30 mg/L to about 60.0 mg/L, 40 mg/L to about 100.0 mg/L, 40 mg/L to about 90.0 mg/L, 40 mg/L to about 80.0 mg/L, 40 mg/L to about 75.0 mg/L, 40 mg/L to about 70.0 mg/L, 40 mg/L to about 60.0 mg/L, including all ranges derivable therebetween. In some embodiments, the concentration of 2iP in the first bud induction medium and/or the second (or extended) bud induction medium may be about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, about 30 mg/L, about 40 mg/L, about 50 mg/L, about 60 mg/L, about 70 mg/L, about 75 mg/L, about 80 mg/L, about 90 mg/L, or about 100 mg/L, including all ranges derivable there between.

In some embodiments, the first bud induction medium and/or the second (or extended) bud induction medium may comprise a cytokinin, wherein the cytokinin is 6-(3-hydroxybenzylamino)purine (meta-topolin). In some embodiments, the concentration of meta-topolin in the first bud induction medium and/or the second (or extended) bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of meta-topolin in the first bud induction medium and/or the second (or extended) bud induction medium may be about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise an auxin, wherein the auxin is 2,4-dichlorophenoxy-acetic acid (2,4-D). In some embodiments, the concentration of 2,4-D in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, including all ranges derivable there between. In some embodiments, the concentration of 2,4-D in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise an auxin, wherein the auxin is 2,4,5-trichloro-phenoxy acetic acid (2,4,5-T). In some embodiments, the concentration of 2,4,5-T in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, including all ranges derivable there between. In some embodiments, the concentration of 2,4,5-T in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise an auxin, wherein the auxin is 4-amino-3,5,6-trichloro-picolinic acid (picloram). In some embodiments, the concentration of picloram in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. In some embodiments, the concentration of picloram in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise an auxin, wherein the auxin is indole-3-acetic acid (IAA). In some embodiments, the concentration of IAA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of IAA in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise an auxin, wherein the auxin is indole-3-butyric acid (IBA). In some embodiments, the concentration of IBA in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. In some embodiments, the concentration of IBA in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

In some embodiments, the first bud induction medium and/or the second (or extended) bud induction medium may comprise an auxin, wherein the auxin is naphthalene acetic acid (NAA). In some embodiments, the concentration of NAA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.2 mg/L to about 25.0 mg/L, about 0.2 mg/L to about 20.0 mg/L, about 0.2 mg/L to about 15.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 25.0 mg/L, about 3.0 mg/L to about 20.0 mg/L, about 3.0 mg/L to about 15.0 mg/L, about 3.0 mg/L to about 12.5 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 25.0 mg/L, about 4.0 mg/L to about 20.0 mg/L, about 4.0 mg/L to about 15.0 mg/L, about 4.0 mg/L to about 12.5 mg/L, about 4.0 mg/L to about 10.0 mg/L, about 4.0 mg/L to about 7.5 mg/L, about 4.0 mg/L to about 7.0 mg/L, about 4.0 mg/L to about 6.0 mg/L, including all ranges derivable there between. In some embodiments, the concentration of NAA in the first bud induction medium and/or the second (or extended) bud induction medium may be 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, or about 20.0 mg/L, including all ranges derivable there between.

In some embodiments, the first bud induction medium and/or the second (or extended) bud induction medium may comprise an auxin, wherein the auxin is 2,3,5-triiodobenzoic acid (TIBA). In some embodiments, the concentration of TIBA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.2 mg/L to about 25.0 mg/L, about 0.2 mg/L to about 20.0 mg/L, about 0.2 mg/L to about 15.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 2.0 mg/L to about 10.0 mg/L, about 2.0 mg/L to about 7.5 mg/L, about 2.0 mg/L to about 7.0 mg/L, about 2.0 mg/L to about 6.0 mg/L, about 3.0 mg/L to about 25.0 mg/L, about 3.0 mg/L to about 20.0 mg/L, about 3.0 mg/L to about 15.0 mg/L, about 3.0 mg/L to about 12.5 mg/L, about 3.0 mg/L to about 10.0 mg/L, about 3.0 mg/L to about 7.5 mg/L, about 3.0 mg/L to about 7.0 mg/L, about 3.0 mg/L to about 6.0 mg/L, about 4.0 mg/L to about 25.0 mg/L, about 4.0 mg/L to about 20.0 mg/L, about 4.0 mg/L to about 15.0 mg/L, about 4.0 mg/L to about 12.5 mg/L, about 4.0 mg/L to about 10.0 mg/L, about 4.0 mg/L to about 7.5 mg/L, about 4.0 mg/L to about 7.0 mg/L, about 4.0 mg/L to about 6.0 mg/L, including all ranges derivable there between. In some embodiments, the concentration of TIBA in the first bud induction medium and/or the second (or extended) bud induction medium may be 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, or about 20.0 mg/L, including all ranges derivable there between.

In some embodiments, the first bud induction medium and/or the second (or extended) bud induction medium may comprise an auxin, wherein the auxin is phenylacetic acid (PAA). In some embodiments, the concentration of PAA in the first bud induction medium and/or the second (or extended) bud induction medium may be in the range from about 0.1 mg/L to about 25.0 mg/L, about 0.1 mg/L to about 20.0 mg/L, about 0.1 mg/L to about 15.0 mg/L, about 0.2 mg/L to about 25.0 mg/L, about 0.2 mg/L to about 20.0 mg/L, about 0.2 mg/L to about 15.0 mg/L, about 0.5 mg/L to about 25.0 mg/L, about 0.5 mg/L to about 20.0 mg/L, about 0.5 mg/L to about 15.0 mg/L, about 0.5 mg/L to about 12.5 mg/L, 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween. In some embodiments, the concentration of PAA in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, about 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, about 10.0 mg/L, about 11.0 mg/L, about 12.0 mg/L, about 13.0 mg/L, about 14.0 mg/L, about 15.0 mg/L, about 16.0 mg/L, about 17.0 mg/L, about 18.0 mg/L, about 19.0 mg/L, about 20.0 mg/L, about 21.0 mg/L, about 22.0 mg/L, about 23.0 mg/L, about 24.0 mg/L, or about 25.0 mg/L, including all ranges derivable there between.

In some embodiments, the first bud induction medium and/or the second (or extended) bud induction medium may comprise an auxin, wherein the auxin is 3,6-dichloro-2-methoxy-benzoic acid (dicamba). In some embodiments, the concentration of dicamba in the first bud induction medium and/or the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. In some embodiments, the concentration of dicamba in the first bud induction medium and/or the second (or extended) bud induction medium may be about 0.1 mg/L, about 0.2 mg/L, about 0.3 mg/L, about 0.4 mg/L, about 0.5 mg/L, about 0.6 mg/L, about 0.7 mg/L, about 0.8 mg/L, about 0.9 mg/L, 1.0 mg/L, about 1.5 mg/L, about 2.0 mg/L, about 2.5 mg/L, about 3.0 mg/L, about 3.5 mg/L, about 4.0 mg/L, about 4.5 mg/L, about 5.0 mg/L, about 6.0 mg/L, about 7.0 mg/L, about 8.0 mg/L, about 9.0 mg/L, or about 10.0 mg/L, including all ranges derivable there between.

According to some embodiments, the first bud induction medium comprises a first auxin and a first cytokinin, wherein the first auxin is 2,4-dichlorophenoxy-acetic acid (2,4-D) and the first cytokinin is 6-benzylaminopurine (BAP). According to these embodiments, the concentration of 2,4-D in the first bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.1 mg/L to about 2.0 mg/L, about 0.1 mg/L to about 1.5 mg/L, about 0.1 mg/L to about 1.25 mg/L, about 0.1 mg/L to about 1.2 mg/L, about 0.1 mg/L to about 1.1 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.2 mg/L to about 2.0 mg/L, about 0.2 mg/L to about 1.5 mg/L, about 0.2 mg/L to about 1.25 mg/L, about 0.2 mg/L to about 1.2 mg/L, about 0.2 mg/L to about 1.1 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 0.5 mg/L to about 2.0 mg/L, about 0.5 mg/L to about 1.5 mg/L, about 0.5 mg/L to about 1.25 mg/L, about 0.5 mg/L to about 1.2 mg/L, about 0.5 mg/L to about 1.1 mg/L, about 0.75 mg/L to about 2.0 mg/L, about 0.75 mg/L to about 1.5 mg/L, about 0.75 mg/L to about 1.25 mg/L, about 0.75 mg/L to about 1.2 mg/L, about 0.75 mg/L to about 1.1 mg/L, about 0.8 mg/L to about 2.0 mg/L, about 0.8 mg/L to about 1.5 mg/L, about 0.8 mg/L to about 1.25 mg/L, about 0.8 mg/L to about 1.2 mg/L, about 0.8 mg/L to about 1.1 mg/L, about 0.9 mg/L to about 2.0 mg/L, about 0.9 mg/L to about 1.5 mg/L, about 0.9 mg/L to about 1.25 mg/L, about 0.9 mg/L to about 1.2 mg/L, about 0.9 mg/L to about 1.1 mg/L, including all ranges derivable there between. According to these embodiments, the concentration of 6-benzylaminopurine (BAP) in the first bud induction medium may be in the range from about 1.0 mg/L to about 25.0 mg/L, about 1.0 mg/L to about 20.0 mg/L, about 1.0 mg/L to about 15.0 mg/L, about 1.0 mg/L to about 12.5 mg/L, about 2.0 mg/L to about 25.0 mg/L, about 2.0 mg/L to about 20.0 mg/L, about 2.0 mg/L to about 15.0 mg/L, about 2.0 mg/L to about 12.5 mg/L, about 5.0 mg/L to about 25.0 mg/L, about 5.0 mg/L to about 20.0 mg/L, about 5.0 mg/L to about 15.0 mg/L, about 5.0 mg/L to about 12.5 mg/L, about 7.5 mg/L to about 25.0 mg/L, about 7.5 mg/L to about 20.0 mg/L, about 7.5 mg/L to about 15.0 mg/L, about 7.5 mg/L to about 12.5 mg/L, about 8.0 mg/L to about 12.0 mg/L, or about 9.0 mg/L to about 11.0 mg/L, including all ranges derivable therebetween.

According to some embodiments, the second (or extended) bud induction medium comprises a second auxin and a second cytokinin, wherein the second auxin is 4-amino-3,5,6-trichloro-picolinic acid (picloram) and the second cytokinin is thidiazuron (TDZ). According to these embodiments, the concentration of picloram in the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between. According to these embodiments, the concentration of TDZ in the second (or extended) bud induction medium may be in the range from about 0.1 mg/L to about 10.0 mg/L, about 0.1 mg/L to about 7.5 mg/L, about 0.1 mg/L to about 7.0 mg/L, about 0.1 mg/L to about 6.0 mg/L, about 0.1 mg/L to about 5.0 mg/L, about 0.1 mg/L to about 4.0 mg/L, about 0.1 mg/L to about 3.0 mg/L, about 0.2 mg/L to about 10.0 mg/L, about 0.2 mg/L to about 7.5 mg/L, about 0.2 mg/L to about 7.0 mg/L, about 0.2 mg/L to about 6.0 mg/L, about 0.2 mg/L to about 5.0 mg/L, about 0.2 mg/L to about 4.0 mg/L, about 0.2 mg/L to about 3.0 mg/L, about 0.5 mg/L to about 10.0 mg/L, about 0.5 mg/L to about 7.5 mg/L, about 0.5 mg/L to about 7.0 mg/L, about 0.5 mg/L to about 6.0 mg/L, about 0.5 mg/L to about 5.0 mg/L, about 0.5 mg/L to about 4.0 mg/L, about 0.5 mg/L to about 3.0 mg/L, about 1.0 mg/L to about 10.0 mg/L, about 1.0 mg/L to about 7.5 mg/L, about 1.0 mg/L to about 7.0 mg/L, about 1.0 mg/L to about 6.0 mg/L, about 1.0 mg/L to about 5.0 mg/L, about 1.0 mg/L to about 5.0 mg/L, about 1.0 mg/L to about 4.0 mg/L, about 1.0 mg/L to about 3.0 mg/L, or about 1.5 mg/L to about 2.5 mg/L, including all ranges derivable there between.

According to embodiments of the present disclosure, the monocot seed embryo explant(s) may be cultured in contact with the first bud induction medium for about 2 days to about 14 days, about 4 days to about 12 days, about 5 days to about 10 days, or about 6 days to about 8 days, including all ranges derivable therebetween. According to some embodiments, the monocot seed embryo explant(s) is/are cultured in contact with the first bud induction medium for about 2 days, about 3 days, about 4 days, about 5 days, about 6 days, about 7 days (or about 1 week), about 8 days, about 9 days, about 10 days, about 11 days, about 12 days, about 13 days, or about 14 days (or about 2 weeks), including all ranges derivable therebetween. In some embodiments, the monocot seed embryo explant(s) may be cultured in contact with the first bud induction medium at a temperature in a range from about 20° C. to about 30° C., about 22° C. to about 28° C., about 25° C. to about 30° C., about 25° C. to about 29° C., or about 25° C. to about 28° C., including all ranges derivable therebetween. According to some embodiments, the monocot seed embryo explant(s) may be cultured in contact with the first bud induction medium at a temperature of about 20° C., about 21° C., about 22° C., about 23° C., about 24° C., about 25° C., about 26° C., about 27° C., about 28° C., about 29° C., or about 30° C., including all ranges derivable therebetween. According to an aspect of the present disclosure, the monocot seed embryo explant(s) in contact with the first bud induction medium at elevated temperature, which may be in a range from about 30° C. to about 40° C., about 30° C. to about 38° C., about 30° C. to about 36° C., about 30° C. to about 35° C., about 31° C. to about 40° C., about 31° C. to about 38° C., about 31° C. to about 36° C., about 31° C. to about 35° C., about 32° C. to about 40° C., about 32° C. to about 38° C., about 32° C. to about 36° C., about 32° C. to about 35° C., about 33° C. to about 40° C., about 33° C. to about 38° C., about 33° C. to about 36° C., or about 33° C. to about 35° C., including all ranges derivable therebetween. According to some embodiments, the monocot seed embryo explant(s) may be cultured in contact with the first bud induction medium at an elevated temperature of about 30° C., about 31° C., about 32° C., about 33° C., about 34° C., about 35° C., about 36° C., about 37° C., about 38° C., about 39° C., or about 40° C., including all ranges derivable therebetween. A selection agent may generally be absent from the first bud induction medium, but the first bud induction medium may alternatively comprise a selection agent.

In another aspect, culturing monocot seed embryo explants in contact with the first bud induction medium at an elevated temperature, for example a temperature in a range from about 30° C. to about 40° C., for about one week may improve transformation by at least about 0.2-fold, 0.4-fold, 0.6-fold, 0.8-fold, 1-fold, 2-fold, 3-fold, 4-fold, 5-fold, 6-fold, 7-fold, 8-fold, 9-fold, 10-fold, 15-fold, 20-fold, 30-fold, or 40-fold, including all ranges derivable therebetween, as compared to culturing the explants in contact with the first bud induction medium at a lower temperature, for example at a temperature in a range from about 20° C. to about 30° C., during the first bud induction step.

The first bud induction step may also be carried out under a variety of lighting conditions. While some degree of lighting may generally be used, all or part of the first bud induction step may alternatively be performed in the dark. According to some embodiments, the first bud induction step may be carried out with an average or set light intensity of Photosynthetic Active Radiation (PAR) in a range from about 0 μ/m²·s to about 200 μ/m²·s, 20 μ/m²·s to about 200 μ/m²·s, 20 μ/m²·s to about 180 μ/m²·s, 30 μ/m²·s to about 180 μ/m²·s, 50 μ/m²·s to about 180 μ/m²·s, 50 μ/m²·s to about 150 μ/m²·s, 60 μ/m²·s to about 150 μ/m²·s, 70 μ/m²·s to about 140 μ/m²·s, 80 μ/m²·s to about 130 μ/m²·s, or 90 μ/m²·s to about 120 μ/m²·s. According to some embodiments, the first bud induction step may be carried out with an average or set light intensity of Photosynthetic Active Radiation (PAR) at about 0 μ/m²·s, about 10 μ/m²·s, about 20 μ/m²·s, about 30 μ/m²·s, about 40 μ/m²·s, about 50 μ/m²·s, about 60 μ/m²·s, about 70 μ/m²·s, about 80 μ/m²·s, about 90 μ/m²·s, about 100 μ/m²·s, about 110 μ/m²·s, about 120 μ/m²·s, about 130 μ/m²·s, about 140 μ/m²·s, about 150 μ/m²·s, about 160 μ/m²·s, about 170 μ/m²·s, about 180 μ/m²·s, about 190 μ/m²·s, or about 200 μ/m²·s. According to some embodiments, different amounts of light and dark cycles may be used during the first bud induction step, which may comprise a presence of lighting for a length of time between about 0 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length.

According to some embodiments, the amounts of light and dark cycles during the first bud induction step may be about 0 hours of light and about 24 hours of dark, about 1 hour of light and about 23 hours of dark, about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

According to embodiments of the present disclosure, the monocot seed embryo explant(s) may be cultured in contact with the second (or extended) bud induction medium for about 4 days to about 28 days, about 4 days to about 25 days, about 4 days to about 21 days, about 5 days to about 25 days, about 5 days to about 23 days, about 7 days to about 21 days, about 5 days to about 15 days, about 7 days to about 14 days, about 12 days to about 23 days, or about 14 days to about 21 days, including all ranges derivable therebetween. According to some embodiments, the monocot seed embryo explant(s) is/are cultured in contact with the second (or extended) bud induction medium for about 4 days, about 5 days, about 6 days, about 7 days (or about 1 week), about 8 days, about 9 days, about 10 days, about 11 days, about 12 days, about 13 days, about 14 days (or about 2 weeks), about 15 days, about 16 days, about 17 days, about 18 days, about 19 days, about 20 days, about 21 days (or about 3 weeks), about 22 days, about 23 days, about 24 days, about 25 days, about 26 days, about 27 days, or about 28 days (or about 4 weeks), including all ranges derivable therebetween. In some embodiments, the monocot seed embryo explant(s) may be cultured in contact with the second (or extended) bud induction medium at a temperature in a range from about 20° C. to about 32° C., about 20° C. to about 30° C., about 22° C. to about 28° C., about 25° C. to about 30° C., about 25° C. to about 29° C., about 26° C. to about 29° C., about 25° C. to about 28° C., or about 27° C. to about 28° C., including all ranges derivable therebetween. According to some embodiments, the monocot seed embryo explant(s) may be cultured in contact with the second (or extended) bud induction medium at a temperature of about 20° C., about 21° C., about 22° C., about 23° C., about 24° C., about 25° C., about 26° C., about 27° C., about 28° C., about 29° C., or about 30° C., including all ranges derivable therebetween. In a particular embodiment, the monocot seed embryo explant may be cultured in contact with a first bud induction medium for a time period in a range from about 2 days to about 14 days at a temperature in a range from about 20° C. to about 30° C. or at an elevated temperature in a range from about 30° C. to about 40° C., and then subsequently cultured in contact with a second (or extended) bud induction medium for a time period in a range from about 4 days to about 28 days at a temperature in a range from about 20° C. to about 32° C. The second (or extended) bud induction medium may also comprise a selection agent. In another embodiment, culturing the explant in contact with the second bud induction medium may improve transformation by at least about 0.2-fold, 0.4-fold, 0.6-fold, 0.8-fold, 1-fold, 2-fold, 3-fold, 4-fold, 5-fold, 6-fold, 7-fold, 8-fold, 9-fold, 10-fold, 15-fold, 20-fold, 30-fold, or 40-fold.

The second (or extended) bud induction step may also be carried out under a variety of lighting conditions. Some degree of lighting may generally be used during the second (or extended) bud induction step. According to some embodiments, the second (or extended) bud induction step may be carried out with an average or set light intensity of Photosynthetic Active Radiation (PAR) in a range from about 30 μ/m²·s to about 200 μ/m²·s, 30 μ/m²·s to about 180 μ/m²·s, 50 μ/m²·s to about 180 μ/m²·s, 50 μ/m²·s to about 150 μ/m²·s, 60 μ/m²·s to about 150 μ/m²·s, 70 μ/m²·s to about 140 μ/m²·s, 80 μ/m²·s to about 130 μ/m²·s, or 90 μ/m²·s to about 120 μ/m²·s. According to some embodiments, the second (or extended) bud induction step may be carried out with an average or set light intensity of Photosynthetic Active Radiation (PAR) at about 10 μ/m²·s, about 20 μ/m²·s, about 30 μ/m²·s, about 40 μ/m²·s, about 50 μ/m²·s, about 60 μ/m²·s, about 70 μ/m²·s, about 80 μ/m²·s, about 90 μ/m²·s, about 100 μ/m²·s, about 110 μ/m²·s, about 120 μ/m²·s, about 130 μ/m²·s, about 140 μ/m²·s, about 150 μ/m²·s, about 160 μ/m²·s, about 170 μ/m²·s, about 180 μ/m²·s, about 190 μ/m²·s, or about 200 μ/m²·s. According to some embodiments, different amounts of light and dark cycles may be used during the second (or extended) bud induction step, which may comprise a presence of lighting for a length of time between about 2 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length.

According to some embodiments, the amounts of light and dark cycles during the second (or extended) bud induction step may be about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

Without being bound by theory, the bud induction step(s) may cause differentiation and/or proliferation of cells of the explant to form multiple buds on the explant, which may then be regenerated into a plant. According to some preferred embodiments, the first auxin and cytokinin are different that than the second auxin and cytokinin to affect the formation of the multiple buds through somewhat activities and/or modes of action. Without being bound by theory, the first bud induction step may cause differentiation of cells of the explant into multiple buds, whereas the second (or extended) bud induction step may greater proliferation or expansion of the multiple buds to produce a more compact or solid multiple bud explant for further culturing and regeneration into a plant that may have the further benefit of reducing chimerism of the resulting genetically modified plant or plant part. According to some embodiments, culturing the monocot seed embryo explant(s) in a first bud induction medium followed by a second (or extended) bud induction medium may reduce chimerism in regenerated plants by at least 10%, at least 20%, at least 30%, at least 40%, at least 50%, at least 60%, at least 70%, at least 80%, or at least 90%, as compared to culturing the monocot seed embryo explant(s) in the first bud induction medium but without culturing in the second (or extended) bud induction medium prior to regeneration.

### E. Regeneration of Transformed Plants

In another aspect of the present disclosure, a genetically modified monocot plant or plant part is regenerated from a cultured monocot seed embryo explant(s) in contact with a regeneration medium. According to present embodiments, a regeneration medium may comprise a variety of standard culture media or solution ingredients or components, such as for example, basal salts, macronutrients, micronutrients, sugars, antibiotics and/or vitamins. The regeneration medium may generally not comprise an auxin or a cytokinin, although an auxin and/or a cytokinin may alternatively be present. The regeneration medium may typically comprise one or more selection agent(s). The regeneration medium may be a solid, semi-solid or liquid medium, although a regeneration media may typically be a solid medium. A solid medium may comprise a gelling or polymeric agent or ingredient, such as agarose, etc., that can solidify and form the solid medium. As used herein, the term “regeneration” refers to the process of growing a plant from one or more plant cells or tissues of an explant, and the term “regeneration medium” refers to a plant tissue culture medium formulated for regeneration of a plant from an explant. In some embodiments, regeneration or a regeneration step may refer to one or more regeneration step(s) that may involve culturing an explant or cultured explant in two or more regeneration media, which may be the same or different regeneration medium/media, such as by subculturing or transferring the explant from a first regeneration medium to a second regeneration medium, and possibly to a third regeneration medium, and so on.

According to many embodiments, the regeneration medium comprises a low salt concentration. As used herein “low salt concentration” refers to a medium comprising total salt concentration that is less than or equal to about 2800 mg/L. As used herein, a “salt” has a commonly understood meaning in the field of chemistry and refers to an ionic chemical compound, or a dissolved chemical compound if present in a solution, comprising at least one cation (or base) and at least one anion (or acid). The regeneration medium may comprise, in some embodiments, a total salt concentration of less than or equal to about 3000 mg/L, 2800 mg/L, about 2700 mg/L, about 2600 mg/L, about 2500 mg/L, about 2400 mg/L, about 2300 mg/L, about 2200 mg/L, about 2100 mg/L, or about 2000 mg/L. In another embodiment, the regeneration medium may comprise a salt concentration of about 1200 mg/L to about 3000 mg/L, about 1200 mg/L to about 2800 mg/L, about 1300 mg/L to about 2700 mg/L, about 1400 mg/L to about 2600 mg/L, about 1500 mg/L to about 2500 mg/L, about 1600 mg/L to about 2400 mg/L, about 1700 mg/L to about 2400 mg/L, about 1800 mg/L to about 2400 mg/L, about 1900 mg/L to about 2400 mg/L, about 2000 mg/L to about 2400 mg/L, about 2100 mg/L to about 2400 mg/L, about 2200 mg/L to about 2400 mg/L, or about 2300 mg/L, including all ranges derivable therebetween. The total nitrogen concentration of the regeneration medium may, in some embodiments, be in a range from about 0.5 mM to about 20 mM, about 0.5 mM to about 10 mM, about 1 mM to about 20 mM, about 5 mM to about 20 mM, about 1 mM to about 15 mM, about 5 mM to about 15 mM, about 1 mM to about 10 mM, about 1 mM to about 7.5 mM, about 2.5 mM to about 7.5 mM, about 5 mM to about 10 mM, about 10 mM to about 15 mM, about 10 mM to about 20 mM, or about 15 mM to about 20 mM, including all ranges derivable therebetween. As used herein, the term “total nitrogen concentration” refers to the total concentration of nitrogen containing ions, such as nitrate and ammonium ions.

A regeneration medium for use according to the methods described herein may be described, in some embodiments, in terms of its nitrate, ammonium, potassium, or sulfate ion concentration. The nitrate ion concentration may be for example about 0.5 mM to about 20 mM, about 5 mM to about 20 mM, about 5 mM to about 15 mM, about 5 mM to about 10 mM, about 10 mM to about 15 mM, about 10 mM to about 20 mM, or about 15 mM to about 20 mM, including all ranges derivable therebetween. The ammonium ion concentration may be for example about 0.5 mM to about 15 mM, about 2.5 mM to about 15 mM, about 2.5 mM to about 10 mM, about 2.5 mM to about 5 mM, about 5 mM to about 15 mM, about 5 mM to about 10 mM, or about 10 mM to about 15 mM, including all ranges derivable therebetween. The potassium ion concentration may be for example about 0.5 mM to about 15 mM, about 2.5 mM to about 15 mM, about 2.5 mM to about 10 mM, about 2.5 mM to about 5 mM, about 5 mM to about 15 mM, about 5 mM to about 10 mM, or about 10 mM to about 15 mM, including all ranges derivable therebetween. The sulfate ion concentration may be for example about 0.5 mM to about 20 mM, about 5 mM to about 20 mM, about 5 mM to about 15 mM, about 5 mM to about 10 mM, about 10 mM to about 15 mM, about 10 mM to about 20 mM, or about 15 mM to about 20 mM, including all ranges derivable therebetween.

The regeneration medium may, in some embodiments, be described by its ammonium nitrate, calcium chloride, calcium nitrate, or potassium sulfate concentration. The concentration of ammonium nitrate may be, for example, in a range from about 100 mg/L to about 1000 mg/L, about 100 mg/L to about 750 mg/L, about 100 mg/L to about 500 mg/L, about 100 mg/L to about 250 mg/L, or about 250 mg/L to about 500 mg/L, including all ranges derivable therebetween. The concentration of calcium chloride may be, for example, less than or equal to about 100 mg/L, greater than or equal to about 50 mg/L, about 50 mg/L to about 100 mg/L, or about 50 mg/L to about 75 mg/L, including all ranges derivable therebetween. The concentration of calcium nitrate may be, for example, less than or equal to about 500 mg/L, about 100 mg/L to about 500 mg/L, about 100 mg/L to about 300 mg/L, about 300 mg/L to about 400 mg/L, or about 100 mg/L to about 200 mg/L, including all ranges derivable therebetween. The concentration of potassium sulfate may be, for example, greater than about 500 mg/L, about 500 mg/L to about 750 mg/L, about 500 mg/L to about 1000 mg/L, about 500 mg/L to about 1500 mg/L, about 500 mg/L to about 2000 mg/L, about 750 mg/L to about 1000 mg/L, or about 1000 mg/L, including all ranges derivable therebetween.

In one aspect, monocot seed embryo explant is regenerated in contact with the regeneration medium at about 20° C. to about 32° C., 25° C. to about 29° C., or about 27° C. to about 28° C., including all ranges derivable therebetween. The monocot seed embryo explant may be regenerated, in some embodiments, for about 20 days to about 50 days or about 28 days to about 42 days, including all ranges derivable therebetween.

In another aspect, regenerating a genetically modified monocot plant or plant part on regeneration medium comprising a low salt concentration may improve transformation by at least about 0.1-fold, 0.2-fold, 0.3-fold, 0.4-fold, 0.5-fold, 0.6-fold, 0.7-fold, 0.8 fold, 0.9-fold, 1-fold, 1.5-fold, 2-fold, 2.5-fold, 3-fold, 3.5-fold, 4-fold, 4.5-fold, or 5-fold compared to genetically modified monocot plants or plant parts regenerated on a regeneration medium comprising a higher salt concentration. In addition, regenerating a genetically modified monocot plant or plant part on medium comprising a low salt concentration may increase rooting frequency by at least about 0.1-fold, 0.2-fold, 0.3-fold, 0.4-fold, 0.5-fold, 0.6-fold, 0.7-fold, 0.8 fold, 0.9-fold, 1-fold, 2-fold, 2.5-fold, 3-fold, 3.5-fold, 4-fold, 4.5-fold, or 5-fold compared to genetically modified monocot plants or plant parts regenerated on regeneration medium comprising higher salt concentration.

The regeneration step may also be carried out under a variety of lighting conditions. Some degree of lighting may generally be used during the second (or extended) bud induction step. According to some embodiments, the regeneration step may be carried out with an average or set light intensity of Photosynthetic Active Radiation (PAR) in a range from about 30 μ/m²·s to about 250 μ/m²·s, about 30 μ/m²·s to about 225 μ/m²·s, about 30 μ/m²·s to about 200 μ/m²·s, about 40 μ/m²·s to about 200 μ/m²·s, about 50 μ/m²·s to about 200 μ/m²·s, 50 μ/m²·s to about 180 μ/m²·s, 60 μ/m²·s to about 180 μ/m²·s, 70 μ/m²·s to about 180 μ/m²·s, 80 μ/m²·s to about 180 μ/m²·s, 90 μ/m²·s to about 180 μ/m²·s, 100 μ/m²·s to about 170 μ/m²·s, 110 μ/m²·s to about 160 μ/m²·s, or about 120 μ/m²·s to about 150 μ/m²·s. According to some embodiments, the second (or extended) bud induction step may be carried out with an average or set light intensity of Photosynthetic Active Radiation (PAR) at about 20 μ/m²·s, about 30 μ/m²·s, about 40 μ/m²·s, about 50 μ/m²·s, about 60 μ/m²·s, about 70 μ/m²·s, about 80 μ/m²·s, about 90 μ/m²·s, about 100 μ/m²·s, about 110 μ/m²·s, about 120 μ/m²·s, about 130 μ/m²·s, about 140 μ/m²·s, about 150 μ/m²·s, about 160 μ/m²·s, about 170 μ/m²·s, about 180 μ/m²·s, about 190 μ/m²·s, about 200 μ/m²·s, about 210 μ/m²·s, about 220 μ/m²·s, about 230 μ/m²·s, about 240 μ/m²·s, or about 250 μ/m²·s. According to some embodiments, different amounts of light and dark cycles may be used during the second (or extended) bud induction step, which may comprise a presence of lighting for a length of time between about 2 hours and about 24 hours of light, about 2 hours and about 22 hours of light, about 4 hours and about 20 hours of light, about 8 hours and about 20 hours of light, about 12 hours and about 20 hours of light, about 16 hours and about 20 hours of light, each with a corresponding amount of relative darkness for a corresponding length of time based on 24-hour day length.

According to some embodiments, the amounts of light and dark cycles during the regeneration step may be about 2 hours of light and about 22 hours of dark, about 3 hours of light and about 21 hours of dark, about 4 hours of light and about 20 hours of dark, about 5 hours of light and about 19 hours of dark, about 6 hours of light and about 18 hours of dark, about 7 hours of light and about 17 hours of dark, about 8 hours of light and about 16 hours of dark, about 9 hours of light and about 15 hours of dark, about 10 hours of light and about 14 hours of dark, about 11 hours of light and about 13 hours of dark, about 12 hours of light and about 12 hours of dark, about 13 hours of light and about 11 hours of dark, about 14 hours of light and about 10 hours of dark, about 15 hours of light and about 9 hours of dark, about 16 hours of light and about 8 hours of dark, about 17 hours of light and about 7 hours of dark, about 18 hours of light and about 6 hours of dark, about 19 hours of light and about 5 hours of dark, about 20 hours of light and about 4 hours of dark, about 21 hours of light and about 3 hours of dark, about 22 hours of light and about 2 hours of dark, about 23 hours of light and about 1 hour of dark, about 24 hours of light and about 0 hours of dark.

In still yet another aspect of the present disclosure, the monocot seed embryo explant and the genetically modified monocot plant or plant part are cultured and regenerated without producing a callus tissue culture.

In one aspect of the present disclosure, the regenerated genetically modified monocot plant or plant part is non-chimeric or has reduced chimerism. As used herein the term “chimeric” or “chimerism” refer to a plant, plant tissue, explant, or the like, which is composed of two genetically different types of tissues or cells with respect to a genetic modification.

### F. Genetically Modified Plants by Genetic Engineering

Various genetic engineering technologies have been developed and may be used by those of skill in the art to introduce transgenic or edited traits into plants. The methods generally involve the delivery of a polynucleotide sequence into a plant cell, which may typically be a heterologous and/or recombinant DNA molecule, which may comprise at least one transgene or expression cassette or an RNA molecule, such as a guide RNA (gRNA) or part of a ribonucleoprotein (RNP), such as a gRNA/site-directed nuclease complex for genome editing. In certain aspects of the present embodiments, traits are introduced into monocot plants via altering or introducing a single genetic locus or transgene into the genome of a plant. Methods of genetic engineering to modify, delete, or insert transgenes, edits, mutations and polynucleotide sequences into the genomic DNA of plants are known in the art. Molecular methods of editing a plant cell genome or endogenous plant gene using a genome editing technique is known in the art. According to present embodiments, a polynucleotide or DNA molecule comprising and/or encoding genome editing tools or machinery, such as a guide RNA, site-directed nuclease and/or template DNA molecule, may be introduced into a plant cell using the methods described herein.

In some embodiments, transformed monocot plants can be created through the site-specific modification of a plant genome. Methods of site directed integration of a transgene or polynucleotide sequence include, for example, utilizing sequence-specific nucleases, such as zinc-finger nucleases (see, for example, U.S. Pat. Appl. Pub. No. 2011/0203012); engineered or native meganucleases; TALE-endonucleases (see, for example, U.S. Pat. Nos. 8,586,363 and 9,181,535); RNA-guided endonucleases, such as those of the CRISPR/Cas systems (see, for example, U.S. Pat. Nos. 8,697,359, 8,771,945 and 9,790,490 and U.S. Pat. Appl. Pub. No. 2014/0068797) and CRISPR-associated transposases or CAST (see, for example US Patent Application Pub. No. 2020/0190487), the entire contents and disclosure of which are incorporated herein by reference. Some embodiments thus relate to utilizing a nuclease or any associated protein to carry out genome modification. This nuclease could be provided heterologously within a donor template DNA for templated-genomic editing or in a separate molecule or vector. A recombinant DNA construct may also comprise a sequence encoding one or more guide RNAs to direct the nuclease to the site within the plant genome to be modified. Further methods for altering or introducing a single genetic locus include, for example, utilizing single-stranded oligonucleotides to introduce base pair modifications in a plant genome (see, for example Sauer et al., Plant Physiol, 170(4):1917-1928, 2016, the entire content and disclosure of which is incorporated herein by reference). Other methods for altering a genetic locus include, for example, utilizing CRISPR/Cas Base-editors or Prime-editors to introduce single or multiple base pair modifications in a plant genome (see, for example, Komor et al., Nature 533, 420-424 (2016); Gaudelli et. al., Nature 551, 464-471 (2017); Komor, et. al., Science Advances 3:(8) (2017), and Rees, et. al., Nat Rev Genet. 2018 December, 19(12):770-788, PCT Patent Application Pub. No. WO 2020/191248, the entire contents and disclosures of which are incorporated herein by reference).

Methods for site-directed alteration or introduction/integration of a heterologous and/or recombinant genetic sequence or transgene are known in the art and include those that utilize sequence-specific nucleases, such as the aforementioned, or complexes of proteins and guide-RNA that cut genomic DNA to produce a double-strand break (DSB) or nick at a genetic locus. As is well-understood in the art, during the process of repairing the DSB or nick introduced by the nuclease enzyme, a donor template, transgene, or expression cassette polynucleotide may become integrated into the genome at the site of the DSB or nick by non-homologous end joining (NHEJ) or by homologous recombination (HR) between the homology arm(s) of the desired sequence and the target sequence. This could result in site-directed integration of all or part of the donor template, transgene or expression cassette polynucleotide into the target site for the nuclease in the genome to create the targeted insertion event. The presence of homology arms in the DNA to be integrated which may promote the adoption and targeting of the insertion sequence or part of the insertion sequence into the plant genome during the repair process through homologous recombination or non-homologous end joining (NHEJ).

In other embodiments, genetic modification of a plant may comprise transformation of a plant, plant part, plant tissue or plant cell to insert a polynucleotide or DNA sequence or transgene into the genome of the plant, plant part, plant tissue or plant cell. Methods for transformation of plants that are known in the art and applicable to many crop species include, but are not limited to, electroporation, microprojectile or particle bombardment, microinjection, PEG-mediated transformation, Agrobacterium-mediated transformation, and other modes of direct DNA uptake. Bacteria known to mediate plant cell transformation include a number of species of bacterial genera, species, and strains that may be assigned to the order Rhizobiales other than Agrobacterium, including but not limited to, bacterial species and strains from the taxonomic families Rhizobiaceae (e.g. Rhizobium spp., Sinorhizobium spp.), Phyllobacteriaceae (e.g. Mesorhizobium spp., Phyllobacterium spp.), Brucellaceae (e.g. Ochrobactrum spp.), Bradyrhizobiaceae (e.g. Bradyrhizobium spp.), and Xanthobacteraceae (e.g. Azorhizobium spp.), among others. According to some embodiments, Agrobacterium-mediated transformation is mediated by Agrobacterium tumefaciens. Targets for such transformation have often been undifferentiated callus tissues, although differentiated tissue also has been used for transient and stable plant transformation. As is well known in the art, other methods for plant transformation may be utilized, for instance as described by Miki et al., (1993, “Procedures for Introducing Foreign DNA into Plants,” in Methods in Plant Molecular Biology and Biotechnology, Glick B. R. and Thompson, J. E. Eds., CRC Press, Inc., Boca Raton, pages 67-88).

In specific embodiments, microprojectile bombardment may be employed to deliver transforming a polynucleotide or DNA molecule, vector, sequence or segment to at least one cell of a plant explant(s). In this method, particles are coated with a polynucleotide or polynucleotide/protein complex and delivered into cells by a propelling force. Exemplary particles may include those comprised of tungsten, platinum, or gold. For bombardment, explants or other target cells may be arranged on solid culture medium. The cells to be bombarded are positioned at an appropriate distance below the projectile stopping plate. A polynucleotide may be delivered into plant cells by acceleration using a biolistics particle delivery system, which may propel particles coated with a DNA or polynucleotide molecule through a screen, such as a stainless steel or Nytex screen, and toward the explants positioned on a surface. The screen may disperse the particles so that they are not delivered to the recipient cells in large aggregates. Microprojectile bombardment techniques are widely applicable and may be used to transform a variety of plant species.

Agrobacterium-mediated or Rhizobiales-mediated transformation of explants is another widely applicable system for introducing heterologous and/or recombinant DNA molecules into plant cells. Modern Agrobacterium transformation vectors are capable of replication in E. coli as well as Agrobacterium, allowing for convenient manipulations (see, e.g., Klee et al., Nat. Biotechnol., 3(7):637-642, 1985). Moreover, recent technological advances in vectors for Agrobacterium-mediated gene transfer have improved the arrangement of genes and restriction sites in the vectors to facilitate the construction of vectors capable of expressing various polypeptide coding genes. The vectors described have convenient multi-linker regions flanked by a promoter and a polyadenylation site for direct expression of inserted polypeptide coding genes. Additionally, Agrobacterium containing both armed and disarmed Ti plasmids can be used for transformation. Agrobacterium-mediated transformation is often the method of choice for many plant species. The use of Agrobacterium-mediated plant integrating vectors to introduce DNA into plant cells is known in the art (see, e.g., Fraley et al., Nat. Biotechnol., 3:629-635, 1985; U.S. Pat. No. 5,563,055).

A number of promoters and expression elements have utility for plant gene expression for any selectable marker, scoreable marker, transgene, or any other gene of agronomic interest. Promoters may include any constitutive promoter, tissue specific promoters, organ specific promoters, inducible promoters, reproductive tissue promoter, developmental stage promoter, viral promoter, etc. Examples of various types of promoters and expression elements are known in art. Expression elements that may be useful for plant gene expression may include, for example, various promoters, enhancers, leaders, 5′ and 3′ untranslated regions, introns, terminators, etc., as known in the art. A selectable or screenable marker or gene of interest may also be fused to a transmit peptide or other targeting sequence. Transport of proteins produced by transgenes to a subcellular compartment such as the chloroplast, vacuole, peroxisome, glyoxysome, cell wall, nucleus, or mitochondrion or for secretion into the apoplast, may be accomplished by means of operably linking the nucleotide sequence encoding a signal or targeting sequence to the 5′ and/or 3′ region of a gene encoding the protein of interest. Targeting sequences at the 5′ and/or 3′ end of the structural gene may determine, during protein synthesis and processing, where the encoded protein is ultimately compartmentalized. The presence of a signal sequence directs a polypeptide to either an intracellular organelle or subcellular compartment or for secretion to the apoplast. Many signal sequences are known in the art. See, for example Becker et al. (Plant Mol. Biol., 20:49, 1992); Knox et al. (Plant Mol. Biol., 9:3-17, 1987); Lerner et al. (Plant Physiol., 91:124-129, 1989); Fontes et al. (Plant Cell, 3:483-496, 1991); Matsuoka et al. (Proc. Natl. Acad. Sci. USA, 88:834, 1991); Gould et al. (J. Cell. Biol., 108:1657, 1989); Creissen et al. (Plant J., 2:129, 1991); Kalderon et al. (Cell, 39:499-509, 1984); Steifel et al. (Plant Cell, 2:785-793, 1990).

Examples of constitutive promoters may include, for example, the cauliflower mosaic virus (CaMV) 35S promoter, which confers constitutive, high-level expression in most plant tissues (see, e.g., Odel et al., Nature, 313:810, 1985), including monocots (see, e.g., Dekeyser et al., Plant Cell, 2:591, 1990; Terada and Shimamoto, Mol. Gen. Genet., 220:389, 1990); a tandemly duplicated version of the CaMV 35S promoter, the enhanced 35S promoter (e35S), the nopaline synthase promoter (An et al., Plant Physiol., 88:547, 1988), the octopine synthase promoter (Fromm et al., Plant Cell, 1:977, 1989); and the figwort mosaic virus (FMV) promoter as described in U.S. Pat. No. 5,378,619 and an enhanced version of the FMV promoter (eFMV) where the promoter sequence of FMV is duplicated in tandem, the cauliflower mosaic virus 19S promoter, a sugarcane bacilliform virus promoter, a commelina yellow mottle virus promoter, and other plant DNA virus promoters known to express in plant cells.

With an inducible promoter, the rate of transcription increases in response to an inducing agent. Any inducible promoter can be used in the instant disclosure. A variety of plant gene promoters that are regulated in response to environmental, hormonal, chemical, and/or developmental signals can be used for expression of an operably linked gene in plant cells, including promoters regulated by (1) heat (Callis et al., Plant Physiol., 88:965, 1988), (2) light (e.g., pea rbcS-3A promoter, Kuhlemeier et al., Plant Cell, 1:471, 1989; maize rbcS promoter, Schaffner and Sheen, Plant Cell, 3:997, 1991; or chlorophyll a/b-binding protein promoter, Simpson et al., EMBO J., 4:2723, 1985), (3) hormones, such as abscisic acid (Marcotte et al., Plant Cell, 1:969, 1989), (4) wounding (e.g., wunl, Siebertz et al., Plant Cell, 1:961, 1989); or (5) chemicals such as methyl jasmonate, salicylic acid, or Safener. It may also be advantageous to employ organ-specific or tissue specific promoters known in the art (e.g., Roshal et al., EMBO J., 6:1155, 1987; Schernthaner et al., EMBO J., 7:1249, 1988; Bustos et al., Plant Cell, 1:839, 1989).

Exemplary polynucleotide or DNA molecules which may be introduced to the monocot plants include, for example, DNA sequences or genes from another species, or even genes or sequences which originate with or are present in the same species but are incorporated into recipient cells by genetic engineering methods rather than classical reproduction or breeding techniques. However, the term “exogenous” is also intended to refer to genes that are not normally present in the cell being transformed, or perhaps not present in the form, structure, location, etc. A polynucleotide may include a DNA molecule or sequence which is already present in the plant cell, is from another plant, is from a different organism, is exogenous or generated externally. A transgene or expression cassette may encode a mRNA and protein or an RNA molecule for suppression, such as a miRNA, siRNA, dsRNA, antisense RNA, inverted repeat RNA, etc. A polynucleotide may be an exogenous, heterologous and/or recombinant polynucleotide or DNA molecule or sequence.

Many hundreds if not thousands of different genes are known and could potentially be introduced into a plant according to the disclosure. Non-limiting examples of particular genes and corresponding phenotypes one may choose to introduce into a monocot plant include one or more genes for insect tolerance, such as a Bacillus thuringiensis (B.t.) gene, pest tolerance such as genes for fungal disease control, herbicide tolerance such as genes conferring glyphosate tolerance, and genes for quality improvements such as yield, nutritional enhancements, environmental or stress tolerances, or any desirable changes in plant physiology, growth, development, morphology or plant product(s). For example, structural genes would include any gene that confers insect tolerance including but not limited to a Bacillus insect control protein gene as described in WO 99/31248, herein incorporated by reference in its entirety, U.S. Pat. No. 5,689,052, herein incorporated by reference in its entirety, U.S. Pat. Nos. 5,500,365 and 5,880,275, herein incorporated by reference in their entirety. In another embodiment, the structural gene can confer tolerance to the herbicide glyphosate as conferred by genes including, but not limited to Agrobacterium strain CP4 glyphosate resistant EPSPS gene (aroA:CP4) as described in U.S. Pat. No. 5,633,435, herein incorporated by reference in its entirety, or glyphosate oxidoreductase gene (GOX) as described in U.S. Pat. No. 5,463,175, herein incorporated by reference in its entirety.

A variety of assays are known in the art and may be used to confirm the presence of an exogenous DNA sequence or transgene in transformed, edited or genetically modified plants. Such assays include, but are not limited to, Southern blotting, Northern blotting, sequencing, PCR, in situ hybridization, ELISA, Western blotting, enzymatic function assays, plant part assays, or by analyzing the phenotype of a regenerated plant.

### G. Culture Media

A variety of tissue culture media are known that, when supplemented appropriately, support plant tissue growth and development, including formation of mature plants from excised plant tissue. As used herein the term “tissue culture media” refers to liquid, semi-solid, or solid media used to support plant growth and development in a non-soil environment. These tissue culture media can either be purchased as a commercial preparation or custom prepared and modified by those of skill in the art. Examples of such media include, but are not limited to those described by Murashige and Skoog, (1962); Chu et al., (1975); Linsmaier and Skoog, (1965); Uchimiya and Murashige, (1962); Gamborg et al., (1968); Duncan et al., (1985); McCown and Lloyd, (1981); Nitsch and Nitsch (1969); and Schenk and Hildebrandt, (1972), or derivations of these media supplemented accordingly. Those of skill in the art are aware that media and media supplements, such as nutrients and plant growth regulators for use in transformation and regeneration are usually optimized for the particular target crop or variety of interest. Tissue culture media may be supplemented with carbohydrates such as, but not limited to, glucose, sucrose, maltose, mannose, fructose, lactose, galactose, and/or dextrose, or ratios of carbohydrates. Reagents are commercially available and can be purchased from a number of suppliers (see, for example Sigma Chemical Co., St. Louis, MO; and PhytoTechnology Laboratories, Shawnee Mission, KS). These tissue culture media may be used to prepare an inoculation, co-culture, bud induction, second induction, or regeneration media and in particular embodiments may comprise a selection agent.

### H. Selectable Markers

In particular embodiments, media for use according to the present disclosure may comprise one or more selection agents and the heterologous polynucleotide molecule for use in the present disclosure may comprise a selectable marker gene, wherein the selectable marker gene provides resistance to the selection agent. As used herein, “selectable marker” or “screenable marker” or “scoreable marker” refers to a nucleic acid sequence whose expression confers a phenotype facilitating identification of cells containing the nucleic acid sequence. Examples of various selectable markers and genes providing resistance to them are disclosed in Miki and McHugh, 2004. Selectable marker genes that may be used include, but are in no way limited to, aroA, EPSPS, aadA, pat, bar, hph (hygromycin B phosphotransferase), DMO (dicamba nomooxygenase) and NPT II. Non-limiting examples of selection agents that may be used according to the present disclosure include glyphosate, glufosinate, phosphinothricin, bromoxynil, bialaphos, dicamba, imidazolinone, sulfonylurea, acetolactate synthase inhibitors, protoporphyrinogen oxidase inhibitors, hydroxyphenyl-pyruvate-dioxygenase inhibitors, antibiotic inhibitors, neomycin, kanamycin, paramomycin, G418, aminoglycosides, spectinomycin, streptomycin, hygromycin B, bleomycin, phleomycin, sulfonamides, gentamycin, streptothricin, chloramphenicol, methotrexate, 2-deoxyglucose, betaine aldehyde, S-aminoethyl L-cysteine, 4-methyltryptophan, D-xylose, D-mannose, and benzyladenine-N-3-glucuronidase. The heterologous polynucleotide molecule for use in the present disclosure may, in some embodiments, comprise two or more selectable marker genes. Selection agents for use in the present disclosure may, in some embodiments, be used alone or as a combination of two or more selection agents. In one embodiment, the embodiments of the present disclosure may be performed in the absence of any selection agent.

According to embodiments of the present disclosure, the insertion sequence of an exogenous polynucleotide or DNA molecule for transformation or genome editing may comprise a plant selectable marker gene to allow for successful selection for, and production of, transformed or transgenic R₀ plants. A plant selectable marker gene or transgene may include any gene conferring tolerance to a corresponding selection agent, such that plant cells transformed with the plant selectable marker transgene may tolerate and withstand the selection pressure imposed by the selection agent. As a result, transformed plant cells of an explant are favored to grow, proliferate, develop, etc., under selection. Although a plant selectable marker gene is generally used to confer tolerance to a selection agent, additional screenable marker gene(s) may also be used in addition to the selectable marker, perhaps also along with a gene of agronomic interest. Such screenable marker genes may include, for example, uidA for β-glucuronidase (GUS; e.g., as described in U.S. Pat. No. 5,599,670, which is hereby incorporated by reference) or gfp for green fluorescent protein and variants thereof (GFP described in U.S. Pat. Nos. 5,491,084 and 6,146,826, both of which are hereby incorporated by reference) or crtB for phytoene synthase (e.g., as described in U.S. Pat. Nos. 8,237,016 and 10,240,165, both of which are hereby incorporated by reference. Additional examples of screenable markers may include secretable markers whose expression causes secretion of a molecule(s) that can be detected as a means for identifying transformed cells.

A plant selectable marker gene may comprise a gene encoding a protein that provides or confers tolerance or resistance to an herbicide, such as glyphosate and glufosinate. Useful plant selectable marker genes known in the art may include those encoding proteins that confer resistance or tolerance to streptomycin or spectinomycin (e.g., aadA, spec/strep), kanamycin (e.g., nptII), hygromycin B (e.g., aph IV), gentamycin (e.g., aac3 and aacC4), and chloramphenicol (e.g., CAT). Additional examples of known plant selectable marker genes encoding proteins that confer herbicide resistance or tolerance include, for example, a transcribable DNA molecule encoding 5-enolpyruvylshikimate-3-phosphate synthase (EPSPS for glyphosate tolerance; e.g., as described in U.S. Pat. Nos. 5,627,061; 5,633,435; 6,040,497; and 5,094,945, all of which are hereby incorporated by reference); a transcribable DNA molecule encoding a glyphosate oxidoreductase and a glyphosate-N-acetyl transferase (GOX; e.g., as described in U.S. Pat. No. 5,463,175; GAT described in U.S. Patent publication No. 20030083480; a transcribable DNA molecule encoding phytoene desaturase (crtI; e.g., as described in Misawa, et al., Plant Journal, 4:833-840 (1993) and Misawa, et al., Plant Journal, 6:481-489 (1994) for norflurazon tolerance, incorporated herein by reference); and the bar gene (e.g., as described in DeBlock, et al., EMBO Journal, 6:2513-2519 (1987) for glufosinate and bialaphos tolerance, incorporated herein by reference).

The insertion sequence of an exogenous DNA molecule may further comprise sequences for removal of one or more transgene(s) or expression cassette(s), such as a plant selectable marker transgene, or any portion or sequence thereof, after successful production and/or confirmation of a transformed plant(s), especially after the transgene or expression cassette is no longer needed. In some embodiments, this may be accomplished by flanking the transgene sequence to be removed, with known or later developed recombination sites (e.g., LoxP sites, FRT sites, etc.) that can be recognized and removed by an endogenous or exogenously provided recombinase enzyme (e.g., Cre, Flp, etc.). The recombinase enzyme may be introduced and expressed in trans, such as by crossing the transformed plant to another plant having the recombinase transgene, to accomplish excision of the transgene. Accordingly, the unwanted sequence element or transgene can be removed once its use or purpose has expired, thus preventing its further expression or transmission in the germ line.

### EXAMPLES

Those of skill in the art will appreciate the many advantages of the methods and compositions provided by the present disclosure. The following examples are included to demonstrate the preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments that are disclosed and still obtain a like or similar result without departing from the spirit and scope of the invention. All references cited herein are incorporated herein by reference to the extent that they supplement, explain, provide a background for, or teach methodology, techniques, or compositions employed herein.

### Example 1

### Transformation of Corn Excised Explants

This example describes a method of Agrobacterium-mediated transformation of embryo explants excised from dry mature corn seeds, including the steps of explant preparation, Agrobacterium inoculation and co-culture, bud induction, extended bud induction, and regeneration of transgenic plants.

### Explant Preparation

Explants were excised from dry mature 01DKD2 corn seeds and stored in sealed pouches at −20° C. The explants were removed from the freezer and allowed to equilibrate to room temperature for at least 30 minutes prior to explant preparation. Explants were surface sterilized with 70% ethanol containing 100 g/L polyethylene glycol (PEG) molecular weight (MW) 800 in an appropriately sized roller bottle, which was agitated by rolling slowly for about 3.5 minutes. Approximately 300 mL of sterilization solution was used for up to about 15,000 corn explants and about 500 mL of sterilization solution was used for about 15,000 to about 30,000 corn explants. Following sterilization, the corn explants and sterilization solution were poured over a large steel strainer. The explants retained on the strainer were rinsed with about 2.5 to about 3.0 L of sterile water to remove any significant remaining sterilization solution and then transferred to a sterile glass beaker.

Following sterilization, the corn explants were floatation enriched to remove debris. Briefly, 500 mL of sterile water was poured into the sterile glass beaker comprising the explants and explants that floated to the surface were collected by pouring into a large strainer. Floatation was repeated several times until no remaining viable explants floated to the surface.

Corn explants collected following the floatation enrichment were transferred into a new sterile glass beaker for rehydration. Briefly, 400 mL of rehydration medium comprising ⅖ strength of B5 macro salts except for CaCl₂, which is ½ strength, 1/10 strength of B5 micro salts and vitamins, 1 g/L potassium nitrate, 30 g/L dextrose, 2.8 mg/L sequestrene, 3.9 g/L 2-(N-morpholino)ethanesulfonic acid (MES), and 0.03 g/L Clearys 3336 WP, pH 5.4 was added to the beaker. The beaker was then covered with aluminum foil and incubated for about 1 to about 2 hours. The components for each of the MS, Gamborg B5 (B5), and Woody Plant Medium (WPM) salts and vitamins are known in the art and are provided together in Table 1. See, for example, Murashige and Skoog (1962) Plant Physiology, 15:473-497; O L Gamborg, et al. (1968) Exp Cell Res., 50(1):151-8; and McCown and Lloyd (1981) HortScience, 16:453-453, the entire content and disclosure of each of which is incorporated herein by reference.

TABLE 1
Components of MS, B5, WPM and LowNitrogen MS salts and vitamins.

| | | | | | | | LowNitrogen | |
|---|---|---|---|---|---|---|---|---|
| | MS | | WPM | | B5 | | MS | |
| Components | mg/L | mM | mg/L | mM | mg/L | mM | mg/L | mM |
| Ammonium | 1650 | 20.6 | 400 | 5.0 | — | — | — | — |
| nitrate | | | | | | | | |
| Ammonium | — | — | — | — | 134 | 1.01 | 463 | 3.5 |
| sulfate | | | | | | | | |
| Boric acid | 6.2 | 0.1 | 6.2 | 0.1 | 3.0 | 0.049 | 6.2 | 0.1 |
| Calcium chloride, | 332.2 | 3.0 | 72.5 | 0.65 | 113.24 | 1.02 | — | — |
| anhydrous | | | | | | | | |
| Calcium chloride, | — | — | — | — | — | — | 300 | 2.04 |
| dihydrate | | | | | | | | |
| Calcium nitrate | — | — | 386 | 3.78 | — | — | — | — |
| Cobalt | 0.03 | 0.0001 | — | — | 0.025 | 0.0001 | — | 0.0001 |
| chloride•6H₂O | | | | | | | | |
| Cupric | 0.03 | 0.0001 | 0.25 | 0.001 | 0.025 | 0.0001 | 0.025 | 0.0001 |
| sulfate•5H₂O | | | | | | | | |
| Na₂ EDTA•2H₂O | 37.3 | 0.1 | 37.3 | 0.1 | 37.3 | 0.1 | 37.3 | 0.1 |
| Ferrous | 27.8 | 0.1 | 27.85 | 0.1 | 27.8 | 0.1 | 27.8 | 0.1 |
| sulfate•7H₂O | | | | | | | | |
| Magnesium | 180.7 | 1.5 | 180.7 | 1.5 | 122.09 | 1.01 | — | — |
| sulfate, | | | | | | | | |
| anhydrous | | | | | | | | |
| Magnesium | — | — | — | — | — | — | 370 | 1.5 |
| sulfate, | | | | | | | | |
| heptahydrate | | | | | | | | |
| Manganese | 16.9 | 0.11 | 22.3 | 0.15 | 10 | 0.066 | 16.9 | 0.11 |
| sulfate•H₂O | | | | | | | | |
| Molybdic acid | 0.25 | 0.001 | 0.25 | 0.001 | 0.25 | 0.001 | 0.25 | 0.001 |
| (Na salt)•2H₂O | | | | | | | | |
| Potassium iodide | 0.83 | 0.005 | — | — | 0.75 | 0.0045 | 0.83 | 0.005 |
| Potassium nitrate | 1900 | 18.8 | — | — | 2500 | 24.73 | 2830 | 27.99 |
| Potassium | 170 | 1.25 | 170 | 1.25 | — | — | 185 | 1.36 |
| phosphate, | | | | | | | | |
| monobasic | | | | | | | | |
| Potassium sulfate | — | — | 990 | 5.68 | — | — | — | — |
| Sodium | — | — | — | — | 150 | 1.25 | — | — |
| phosphate | | | | | | | | |
| monobasic | | | | | | | | |
| Zinc sulfate•7H₂O | 8.6 | 0.03 | 8.6 | 0.03 | 2 | 0.007 | 8.6 | 0.03 |
| | | | | | | | — | |
| Myo-inositol | 100 | | 100 | | 100 | | 100 | |
| Nicotinic acid | 0.5 | | 0.5 | | 1 | | 1 | |
| (free acid) | | | | | | | | |
| Pyridoxine•HCl | 0.5 | | 0.5 | | 1 | | 1 | |
| Thiamine•HCl | 0.1 | | 1 | | 10 | | 10 | |
| Glycine (free base) | 2 | | 2 | | — | | — | |
| Total Salt (mg/L) | 4330.83 | | 2301.95 | | 3100.48 | | 4245.93 | |

In Table 1, the major salts or macronutrients are in bold, the minor salts or micronutrients are in normal font, and the vitamins are in italics. Each of these salt and vitamin mixtures are available commercially, such as from PhytoTechnology Laboratories (for example, MS salt mixture catalog number is M519, B5 salt mixture catalog number is G398, WPM salt mixture catalog number is L449, and Low Nitrogen MS mixture catalog numbers are a combination of M529 and G249). The basal salt mixture includes both macronutrients and micronutrients. The concentrations are provided in solution or when a specified amount is added per 1 L total volume water, which may be adjusted for any other volume. Dilutions are made relative to these full concentrations and may be specified as a fraction. The full concentration of salts or vitamins in Table 1 is included in a solution described herein unless a dilution is specified.

### Agrobacterium Preparation

Agrobacterium glycerol stock AB32 was retrieved from a −80° C. freezer and thawed at ambient temperature in a laminar flow hood. After thorough mixing by vortexing, 250 mL of the thawed Agrobacterium glycerol stock was inoculated into 250 mL of liquid LB medium containing appropriate antibiotics for the transformation constructs in a sterile 1 L flask. Appropriate antibiotics may include, for example 50 mg/L spectinomycin or 30 mg/L gentamicin. The flask was placed into an orbital shaker/incubator set to 200 rpm and cultured at 27.5±2° C. in the dark for 16-24 hours, or until the optical density measurement at 660 nm (OD₆₆₀) of the inoculum was within a range of 0.6-1.2. Following centrifugation of the overnight Agrobacterium culture at approximately 3000 rpm or 2620×g (Sorvall® 3B, 6000A rotor) at 4° C. for 25 minutes, the pellet was resuspended in 50 mL of inoculation medium comprising ⅖ strength of B5 macro salts, 1/10 of B5 micro salts and vitamins, 1 g/L potassium nitrate, 30 g/L dextrose, and 3.9 g/L MES. Following resuspension, Agrobacterium from all tubes were pooled and mixed well and a 1:20 dilution of the Agrobacterium suspension was made for OD₆₆₀ measurement. The concentrated Agrobacterium suspension was then diluted to a final OD₆₆₀ of 0.25 in the inoculation medium.

### Inoculation and Co-Culture

Following rehydration as described above, explants were collected by pouring the explant/rehydration mixture into a sterile stainless steel mesh strainer. The retained explants were quickly blotted onto a sterile towel to remove excess liquid. Approximately 2,500 corn explants and 40 ml of Agrobacterium suspension were added to a 50 mL vented conical tube. The Agrobacterium suspension covered all explants in the tube. The tube was then placed in a vacuum chamber where about 300 psi of pressure was applied for about 3 minutes. After the pressure was slowly released, the tubes were centrifuged at 2,620×g at 4° C. for 30 minutes. Following inoculation with pressure and centrifugation, the Agrobacterium suspension was removed by transferring the explants into a sterile stainless-steel strainer. Excess liquid was removed by tapping the strainer onto sterile paper towels.

The inoculated corn explants were transferred to co-culture plates (25 mm×100 mm) containing a piece of sterile Whatman #1 filter paper (82 mm) wetted with 1.25 mL of rehydration medium. Each plate contained approximately 500-600 corn explants evenly spread out across the plate in a single layer. The co-culture plates were incubated at 20° C. and about 65% relative humidity with a photoperiod of 16 hours light/8 hours dark for 5-6 days. The targeted light intensity was about 90 μ/m²·s of Photosynthetic Active Radiation (PAR).

### Bud Induction

Following the co-culture step, corn explants were transferred to a solid bud induction medium, for instance comprising MS salts, B5 vitamins, 30 g/L sucrose, 0.69 g/L proline, 1 g/L NZ amine-A (casein enzymatic hydrolysate; Millipore Sigma), 2 mg/L glycine, 1 g/L MES, 1 mg/L 2,4-dichlorophenoxyacetic acid (2,4-D), 10 mg/L 6-benzylaminopurine (BAP), 400 mg/L carbenicillin, 200 mg/L cefotaxime, 100 mg/L timentin, and 3.5 g/L low EEO agarose, pH 5.8. Explants from each co-culture plate were evenly plated onto five plates containing the bud induction medium. The plates were incubated in a Percival® chamber at 33° C. (35° C. actual plate temperature) with a photoperiod of 16 hours light/8 hours dark for 6-8 days at a light intensity of about 90 μ/m²·s of PAR.

### Extended Bud Induction

Following the bud induction step, corn explants were transferred to a solid extended bud induction medium comprising MS salts, B5 vitamins, 60 g/L sucrose, 0.5 g/L glutamine, 0.69 g/L proline, 1 g/L NZ amine-A, 2 mg/L glycine, 1.95 g/L MES, 1.25 mg/L cupric sulfate, 2 mg/L thidiazuron, 2 mg/L picloram, 400 mg/L carbenicillin, 200 mg/L cefotaxime, 100 mg/L timentin, 25 μM glyphosate and 3.5 g/L low EEO agarose, pH 5.8. Explants from each bud induction plate were divided evenly onto two plates containing the extended bud induction medium. These plates were cultured at 28° C., with a photoperiod of 16 hours light/8 hours dark for about 5-18 days at a light intensity of about 150 μ/m²·s of PAR.

### Regeneration

Explants from the extended bud induction plates were transferred to a solid regeneration medium comprising LM Woody Plant Medium (WPM) salts and vitamins, 0.03 g/L Cleary 3336 WP (thiophanate-methyl), 30 g/L sucrose, 0.69 g/L proline, 2 mg/L glycine, 1 g/L MES, 3.5 g/L low EEO agarose, 400 mg/L carbenicillin, 200 mg/L cefotaxime, 100 mg/L timentin and 20 μM glyphosate, pH 5.8 in 9 cm Vivi® trays (Vivi®, The Netherlands) for shoot development and rooting. After the explants were evenly distributed on the regeneration medium, the Vivi® trays were sealed with plastic film using a hand-held sealing iron and cultured at 28° C. and ambient humidity with a photoperiod of 16 hours light/8 hours dark for 28-42 days at a light intensity of about 160 μ/m²·s of PAR. Following the regeneration step, typically about 10-11 weeks after the inoculation step, putative transgenic plants with visible roots and no sign of chimerism were transplanted in soil plugs (2″ diameter×3″ height (Gro-Tech, Rough and Ready, CA 95975) for further growth and development.

### Example 2

### Transformation of Wheat Excised Explants

This example describes a method of Agrobacterium-mediated transformation of embryo explants excised from dry mature wheat seeds, including the steps of explant preparation, Agrobacterium inoculation and co-culture, bud induction, extended bud induction, and regeneration of transgenic plants.

### Explant Preparation

Explants were excised from dry mature wheat seeds and were stored in sealed pouches at −20° C. The explants were removed from the freezer and allowed to equilibrate to room temperature for at least 30 minutes prior to explant preparation. About 10,000 explants were surface sterilized by placing the explants into a sterile 1 L bottle with about 500 mL of 70% ethanol. The bottle was agitated by rolling or shaking gently by hand for about 5 minutes. Following surface sterilization and removal of the 70% ethanol solution, wheat explants were rinsed with sterile water to remove any significant remaining sterilization medium.

The wheat explants were floatation enriched to remove debris. Briefly, explants in sterile water were poured into a sterile sieve/collection vessel apparatus including an outside container and an inner sieve container. The inner sieve container fits inside the outside container and has a 26×26 stainless steel mesh melted onto the bottom. After the explants were transferred into the apparatus, sterile water was added. The inner sieve container was raised out of and lowered into the water repeatedly to induce floatation of the wheat explants. Floating explants were collected using a vacuum collector. This floatation step was repeated several times until about 50% of the floating material was debris. Following the floatation step, debris was discarded. The collected wheat explants were then added to 50 mL centrifuge tubes using a sterile spatula for Agrobacterium inoculation. About 5,000 wheat explants were added per tube.

### Agrobacterium Preparation

Agrobacterium glycerol stock was retrieved from a −80° C. freezer and thawed at ambient temperature in a laminar flow hood. After thorough mixing by vortexing, 500 mL of the thawed Agrobacterium glycerol stock was inoculated into 250 mL of liquid LB medium containing appropriate antibiotics for the transformation constructs in a 1 L sterile flask on a shaker at 170 rpm and cultured at 27-28° C. in the dark for about 16-18 hour, or until the OD₆₆₀ was within a range of 0.6-1.6. Appropriate antibiotics may include, for example, 50 mg/L spectinomycin or 30 mg/L gentamicin. Following centrifugation of the overnight Agrobacterium culture at approximately 3500-3700 rpm at 20° C. for 20 minutes, the pellet was resuspended in 25 mL of inoculation medium comprising 1/10 of MS salts, MS vitamins, 0.5 g/L glutamine, 0.1 g/L casein hydrolysate. 0.75 g/L magnesium chloride, 1.95 g/L MES, 40 g/L maltose and 100 mg/L ascorbic acid, pH 5.8. Following resuspension, Agrobacterium from all centrifuge tubes were pooled and mixed well and a 1:20 dilution of the Agrobacterium suspension was made for an OD₆₆₀ measurement. The concentrated Agrobacterium suspension was then diluted to a final OD₆₆₀ of 0.5 in the inoculation medium. The prepared Agrobacterium suspension was stored at 4° C. for up to 8 hours until use in transformation.

### Inoculation and Co-Culture

About 40 to 50 mL of the Agrobacterium suspension prepared as described above was added to each 50 mL centrifuge tube containing the prepared wheat explants. The tubes containing the Agrobacterium and the explants were centrifuged at 1400×g at 4° C. for 30 min. Following centrifugation, the explants and Agrobacterium were resuspended by shaking, and the explants were allowed to settle down to the bottom of the tubes. The Agrobacterium suspension was removed by decanting. The inoculated wheat explants were then transferred to a 100 mm×25 mm Petri plate using a sterile spatula or loop and the remaining Agrobacterium suspension was removed using a sterile transfer pipette. Alternatively, a sterile stainless-steel sieve may be used to collect the explants and to remove the excess Agrobacterium suspension as described in Example 1.

The inoculated explants were then transferred to co-culture plates (25 mm×100 mm) containing a piece of sterile Ahlstrom filter paper wetted with 1.25 mL of inoculation medium. Each co-culture plate contained approximately 500-600 wheat explants evenly spread out across the plate in a single layer. The co-culture plates were incubated at 23° C. and about 70% relative humidity in the dark for about 70% for 3-4 days.

### Delay

After the co-culture step, the filter paper with explants were lifted from the co-culture plates using sterile forceps and directly transferred to plates containing solid delay medium comprising 0.78 g/L MS basal salts with no nitrogen, MS vitamins, 1.64 g/L potassium sulfate, 4.95 g/L ammonium nitrate, 60 g/L maltose, 0.5 g/L glutamine, 1 g/L NZ amine-A, 0.75 g/L magnesium chloride hexahydrate, 1.95 g/L MES, 1.25 mg/L cupric sulfate, 3 mg/L thidiazuron, 2 mg/L picloram, 200 mg/L carbenicillin, 100 mg/L cefotaxime and 3.5 g/L agarose low EEO, pH 5.8. The plates were then cultured at 25° C. and about 35% relative humidity with a photoperiod of 16 hours light/8 hours dark for 12-16 days.

### Selection

Following the delay step, selection was carried out using a liquid medium. Briefly, wheat explants on delay medium were transferred to selection plates containing 2 pieces of felt, 1 piece of filter paper with a hole punched through the paper to assist with aspiration, and 25 mL of selection medium comprising 0.78 g/L MS basal salts with no nitrogen, MS vitamins, 1.64 g/L potassium sulfate, 4.95 g/L ammonium nitrate, 30 g/L maltose, 0.5 g/L glutamine, 1 g/L NZ amine-A, 0.75 g/L magnesium chloride, 1.95 g/L MES, 1.25 mg/L cupric sulfate, 3 mg/L thidiazuron, 2 mg/L picloram, 200 mg/L carbenicillin, 100 mg/L cefotaxime and 30 μM glyphosate, pH 5.8. Approximately 80-100 wheat explants were placed into each selection plate. The plates were cultured at 25° C. and a relative humidity of about 35% with a photoperiod of 16 hours light/8 hours dark for 12-16 days.

### Regeneration

Following the selection step, the selection medium was aspirated and 20 mL of liquid regeneration medium comprising MS basal salts, MS vitamins, 30 g/L sucrose, 0.69 g/L proline, 1 g/L MES, 400 mg/L carbenicillin, 200 mg/L cefotaxime, 100 mg/L timentin and 30 μM glyphosate, pH 5.8 was added. The plates were cultured at 25° C. and about 35% relative humidity with a photoperiod of 16 hours light/8 hours dark for 12-16 days (Regeneration Step 1). The regeneration medium was then aspirated and replaced with 15 mL of fresh regeneration medium. The plates were then incubated at 25° C. and about 35% relative humidity with a photoperiod of 16 hours light/8 hours dark for an additional 5-9 days (Regeneration Step 2).

Following Regeneration Step 2, 10 mL of fresh regeneration medium was added to each plate. The plates were then cultured at 25° C. and about 35% relative humidity with a photoperiod of 16 hours light/8 hours dark until regenerated shoots were ready to be transplanted.

Typically, at about 60-70 days post inoculation, regenerated shoots were transferred to solid rooting medium comprising MS basal salts, MS vitamins, 1.95 g/L MES, 40 g/L maltose, 0.5 mg/L cupric sulfate, 100 mg/L ascorbic acid, 1 mg/L IBA, 3 g/L Gelzan CM, 400 mg/L carbenicillin, 500 mg/L cefotaxime and 30 μM glyphosate, pH 5.8 and cultured at 25° C. with a photoperiod of 16 hours light/8 hours dark for 2 weeks. Putative transgenic wheat plants with developed roots were transplanted in soil plugs (2″ diameter×3″ height (Gro-Tech, Rough and Ready, CA 95975) for further growth and development.

### Example 3

### Elevated Temperatures During Bud Induction Improve Transformation of Corn Seed Explants

This example provides experiments designed to evaluate the effect of elevated temperatures during multiple bud induction on the transformation of corn seed excised explants.

### Comparison of Bud Induction for Two Weeks at 28° C. Vs One-Week at 35° C. Followed by One-Week at 28° C.

Explants excised from corn seeds can be placed into contact with bud induction medium and cultured at 28° C. for two weeks following Agrobacterium inoculation and co-culture.

To evaluate the effect of elevated temperature during the initial bud induction step on transformation, two temperature treatments were evaluated using glyphosate for selection: 1) bud induction for two weeks at 28° C.; and 2) bud induction for one week at an elevated temperature of 35° C. followed by one week of bud induction at 28° C. The proceeding and subsequent steps for transformation of the corn explants in this experiment were generally performed as described in Example 1.

Following Agrobacterium inoculation and co-culture, corn explants were transferred to bud induction medium containing MS salts and B5 vitamins supplemented with 2 mg/L TDZ, 1 mg/L 2,4-D and different concentrations of glyphosate and cultured under the temperature conditions described above. As demonstrated in Table 2, culturing the explants at 35° C. for the first week of bud induction appeared to inhibit explant germination, however, after the second week of bud induction at 28° C., the percentage of explants with green bud formation was increased. Furthermore, culturing the explants at an elevated temperature for the first week of bud induction resulted in a significant increase in the frequency of explants producing normal green shoots after 4 weeks of regeneration. This resulted in an increase in shoot frequency and a decrease in chimeric shoot frequency (Table 2). In addition, the results also demonstrated that percentage of normal shoots generally increased as the glyphosate concentration in the selection medium increased from 12.5 μM to 50 μM.

TABLE 2
Elevated temperature during the first week of bud
induction increases transgenic shoot regeneration.

| 1st Week | | | | | | Normal |
|---|---|---|---|---|---|---|
| Bud | Glyphosate | | Shoot | Total # | Normal | Shoot |
| Induction | Concentration | Total # | Frequency | Normal | Shoots | Frequency |
| Temperature | (μM) | Shoots | (%) | Shoots | (%) | (%) |
| 28° C. | 12.5 | 88 | 3.52 | 9 | 10.23 | 0.36 |
| | 25.0 | 22 | 0.88 | 7 | 31.82 | 0.28 |
| | 37.5 | 6 | 0.24 | 5 | 83.33 | 0.2 |
| | 50.0 | 6 | 0.24 | 4 | 66.67 | 0.16 |
| 35° C. | 12.5 | 194 | 7.76 | 28 | 14.43 | 1.12 |
| | 25.0 | 220 | 8.8 | 46 | 20.91 | 1.84 |
| | 37.5 | 149 | 5.96 | 34 | 22.82 | 1.36 |
| | 50.0 | 138 | 5.52 | 45 | 32.61 | 1.8 |

Total number shoots was calculated as the total number of shoots regenerated from inoculated explants, including both normal and chimeric shoots. The shoot frequency was calculated as the number of inoculated explants producing shoots divided by the total number of inoculated explants. Number normal shoots was calculated as the total number of normal shoots regenerated that did not show a chimeric tissue phenotype. The percentage normal shoots was calculated as the number of normal shoots divided by the total number of shoots. Normal shoot frequency was calculated as the number of normal shoots divided by the total number of inoculated explants.

In another experiment, various temperature conditions during the bud induction step were evaluated to compare: 1) 35° C. for the first week of bud induction followed by 28° C. for the second week of bud induction; and 2) two weeks of bud induction at 28° C. This experiment included two different batches of corn explants and different combinations of plant growth regulators in the bud induction media. The following plant growth regulator combinations were evaluated: 1) 2 mg/L TDZ and 1 mg/L 2,4-D; and 2) 10 mg/L BAP and 1 mg/L 2,4-D. As described above, the bud induction medium contained MS salts and B5 vitamins supplemented with either 2 mg/L TDZ, 1 mg/L 2,4-D or 10 mg/L BAP and 1 mg/L 2,4-D, and 5 μM glyphosate as a selection agent. Following Agrobacterium inoculation and co-culture as previously described, the explants were cultured according to the conditions described above. Following co-culture, there was no obvious difference in the transient expression between the two batches of explants. The results of this experiment confirmed that performing the first week of bud induction at 35 NC appears to initially slow the growth of the corn explants and make them more compact and greener compared to the explants cultured at 28° C. for the entire two weeks of bud induction. The results were independent of the explant batch or the plant growth regulator combination in the bud induction medium. The growth of the explants initially cultured at 35° C. recovered during the subsequent bud induction at 28° C. These explants produced a greater percentage of green explants compared to those cultured at 28° C. for two weeks after the explants were transferred to regeneration and selection media and grown for 2, 3 or 4 weeks. Furthermore, as demonstrated in Table 3, explants initially cultured at 35° C. demonstrated an increased shoot frequency and percentage of normal shoots, independent of the explant batch or the plant growth regulator combination in the bud induction medium.

TABLE 3
Elevated temperature during the first week of bud induction
increased transgenic shoot regeneration independent of
explant batch or plant growth regulator combination.

| | 1st Week | | | | | | |
|---|---|---|---|---|---|---|---|
| | Bud | | | | | | Normal |
| | Induction | Bud | | Shoot | Total # | Normal | Shoot |
| Explant | Temperature | Induction | Total # | Frequency | Normal | Shoots | Frequency |
| Batch | (° C.) | Medium | Shoots | (%) | Shoots | (%) | (%) |
| 1 | 28 | 2 mg/L | 47 | 1.88 | 9 | 19.15 | 0.36 |
| | | TDZ, | | | | | |
| | | 1 mg/L | | | | | |
| | | 2,4-D | | | | | |
| | | 10 mg/L | 50 | 2.00 | 11 | 22.00 | 0.44 |
| | | BAP, | | | | | |
| | | 1 mg/L | | | | | |
| | | 2,4-D | | | | | |
| | 35 | 2 mg/L | 77 | 3.08 | 16 | 20.78 | 0.64 |
| | | TDZ, | | | | | |
| | | 1 mg/L | | | | | |
| | | 2,4-D | | | | | |
| | | 10 mg/L | 153 | 6.12 | 38 | 24.84 | 1.52 |
| | | BAP, | | | | | |
| | | 1 mg/L | | | | | |
| | | 2,4-D | | | | | |
| 2 | 28 | 2 mg/L | 41 | 1.64 | 11 | 26.83 | 0.44 |
| | | TDZ, | | | | | |
| | | 1 mg/L | | | | | |
| | | 2,4-D | | | | | |
| | | 10 mg/L | 69 | 2.76 | 52 | 24.64 | 0.68 |
| | | BAP, | | | | | |
| | | 1 mg/L | | | | | |
| | | 2,4-D | | | | | |
| | 35 | 2 mg/L | 65 | 2.60 | 11 | 16.92 | 0.44 |
| | | TDZ, | | | | | |
| | | 1 mg/L | | | | | |
| | | 2,4-D | | | | | |
| | | 10 mg/L | 182 | 7.28 | 44 | 24.18 | 1.76 |
| | | BAP, | | | | | |
| | | 1 mg/L | | | | | |
| | | 2,4-D | | | | | |

Total number shoots was calculated as the total number of shoots regenerated from inoculated explants, including both normal and chimeric shoots. The shoot frequency was calculated as the number of inoculated explants producing shoots divided by the total number of inoculated explants. Number normal shoots was calculated as the total number of normal shoots regenerated that did not show a chimeric tissue phenotype. The percentage normal shoots was calculated as the number of normal shoots divided by the total number of shoots. Normal shoot frequency was calculated as the number of normal shoots divided by the total number of inoculated explants.

### Comparison of Bud Induction at 28° C. Vs. 35° C. Followed by Extended Bud Induction

Three independent experiments were performed using three different batches of corn explants, to evaluate bud induction at 35° C. for the first week followed by 28° C. for the second week compared to bud induction for two weeks at 28° C. prior to the additional extended bud induction, selection, and regeneration steps as described above. Each experiment included four temperature conditions as provided in Table 4.

TABLE 4
Culturing explants at an elevated temperature during bud induction
dramatically increased transgenic shoot regeneration.

| | Bud | Bud | Extended Bud | | |
|---|---|---|---|---|---|
| | Induction | Induction | Induction | Selec- | |
| Experiment | Temperature | Duration | Temperature | tion | NPF |
| Treatment | (° C.) | (week) | (° C.) | Medium | (%) |
| 1-1 (control) | 28 | 2 | — | Liquid | 0.40 |
| 1-2 | 35 + 28 | 1 + 1 | 28 | Solid | 1.40 |
| 1-3 | 35 + 28 | 1 + 1 | 28 | Liquid | 1.60 |
| 1-4 | 28 | 2 | 28 | Liquid | 0.28 |
| 2-1 (control) | 28 | 2 | — | Liquid | 0.39 |
| 2-2 | 35 + 28 | 1 + 1 | 28 | Solid | 1.29 |
| 2-3 | 35 + 28 | 1 + 1 | 28 | Liquid | 1.68 |
| 2-4 | 28 | 2 | 28 | Liquid | 0.68 |
| 3-1 (control) | 28 | 2 | — | Liquid | 0.60 |
| 3-2 | 35 + 28 | 1 + 1 | 28 | Solid | 1.25 |
| 3-3 | 35 + 28 | 1 + 1 | 28 | Liquid | 1.65 |
| 3-4 | 28 | 2 | 28 | Liquid | 1.44 |

As shown in Table 4, culturing explants at 35° C. for the 1 st week of bud induction followed by culturing at 28° C. for a second week of bud induction dramatically increased transgenic shoot regeneration as compared to bud induction at 28° C. for two weeks in all three experiments. The normal plant frequency (NPF) was calculated as the number of normal plants divided by the total number of inoculated explants.

### Bud Induction at 35° C. for 4 Days Followed by Bud Induction at 28° C. for the Remaining Period of Bud Induction was Sufficient to Increase Transgenic Shoot Regeneration as Compared to Bud Induction at 28° C. for 2 Weeks

The effect of various durations of bud induction at 35° C. on transformation was further evaluated. Four independent experiments were conducted with 2 weeks of bud induction at 28° C. (i.e., 0 days at 35° C.) serving as a control. The treatment conditions included bud induction at 35° C. for 2, 4 or 7 days before transferring to 28° C. for the remaining two-week period (i.e., 2 days+12 days; 4 days+10 days; or 7 days+7 days). As shown in Table 5, explants cultured at 35° C. for 4 days during the first week of bud induction had a significant increase in transgenic shoot regeneration as compared to explants cultured at 28° C. throughout the bud induction step. In addition, the average shoot regeneration frequency and average normal shoot frequency for the bud induction treatment at 35° C. for 4 days was comparable to the bud induction treatment at 35° C. for 7 days, indicating that bud induction at an elevated temperature of 35° C. for 4 days is sufficient for the transformation improvement.

TABLE 5
Effect of 35° C. bud induction for various
durations on transgenic shoot regeneration frequency.

| Duration at | # | Total # of | Shoot | Normal Shoot |
|---|---|---|---|---|
| 35° C. (Day) | Replication | Explants | Frequency (%) | Frequency (%) |
| 0 | 4 | 10,000 | 2.23 | 0.38 |
| 2 | 4 | 10,000 | 2.53 | 0.4 |
| 4 | 4 | 10,000 | 5.39 | 0.79 |
| 7 | 4 | 10,000 | 5.7 | 0.63 |

Shoot frequency was calculated as the number of explants producing shoots divided by the total number of explants on bud induction medium for the respective treatment, averaged over the four replicates for each treatment. Normal shoot frequency was calculated as the number of normal shoots divided by the total number of explants on bud induction medium for the respective treatment, averaged over the four replicates for each treatment.

### Example 4

### Regeneration Media Containing Low Levels of Salt Improves Corn Seed Explant Transformation

This example describes improvement in the transformation and rooting frequency of transgenic plants regenerated from corn excised explants contacted with media containing low levels of salt.

During the production of transgenic plants, it is desirable to directly transfer rooted transgenic shoots in contact with regeneration medium from the petri dish to soil, a process known as direct plugging, to eliminate the intermediate steps of growing plants or shoots in a Phytatray™ or PlantCon™ container. Direct plugging requires fewer resources, accelerates the transgenic plant production process, and improves transgenic plant quality. Corn explants can be regenerated by contacting the explants with MS regeneration media following the inoculation, co-culture, bud induction, and extended bud induction steps. The rooting frequency of shoots, however, is often low and variable when MS regeneration media is used, which impedes effective implementation of direct plugging. MS-based media contain high salt levels and therefore experiments were designed to evaluate whether other regeneration media, which contain lower salt levels, improve transformation and/or rooting frequency. The regeneration media evaluated were MS regeneration medium, B5 regeneration medium, Woody Plant Medium (WPM) regeneration medium, and a low nitrogen MS regeneration medium.

Agrobacterium comprising a transformation vector containing a chloroplast-targeted Agrobacterium aroA gene, an uidA gene, and flanking T-DNA borders were prepared as described in Example 1. The aroA gene is a selectable marker that confers resistance to glyphosate and the uidA gene encodes β-glucuronidase (GUS). Following Agrobacterium inoculation and co-culture, the corn explants were placed onto bud induction medium and cultured at 35° C. for one week followed by bud induction at 28° C. for an additional one week. The explants were then transferred to an extended bud induction medium and cultured for an additional two weeks before being placed onto either MS, B5, WPM, or a low nitrogen MS regeneration medium. The components of MS medium, B5 medium, WPM medium, and a low nitrogen MS medium are known in the art and are provided together in Table 1. As shown in Table 6, explants regenerated in contact with WPM regeneration medium demonstrated improved transformation as measured by the normal plant frequency (NPF) compared to explants regenerated in contact with MS regeneration medium.

TABLE 6
WPM regeneration medium improves the normal
plant frequency of transgenic corn plants.

| | | Shoot | Total # | Normal Shoot | |
|---|---|---|---|---|---|
| Regeneration | Total # | Frequency | Normal | Frequency | NPF |
| Medium | Shoot | (%) | Shoots | (%) | (%) |
| MS | 131 | 5.2 | 57 | 2.28 | 1.84 |
| B5 | 113 | 4.5 | 38 | 1.52 | 1.24 |
| WPM | 185 | 7.4 | 74 | 2.96 | 2.68 |
| Low Nitrogen MS | 166 | 6.6 | 76 | 3.04 | 2.24 |

Total number shoots was calculated as the total number of shoots regenerated from inoculated explants, including both normal and chimeric shoots. The shoot frequency was calculated as the number of inoculated explants producing shoots divided by the total number of inoculated explants. Number normal shoots was calculated as the total number of normal shoots regenerated that did not show a chimeric tissue phenotype. The normal shoot frequency was calculated as the number of normal shoots divided by the total number of inoculated explants. The normal plant frequency (NPF) was calculated as the number of normal plants divided by the total number of inoculated explants.

In another experiment, a different batch of corn excised explants and Agrobacterium containing a different transformation vector construct were used to evaluate the transformation and/or rooting frequency of shoots regenerated in contact with WPM regeneration medium compared to MS regeneration medium. The transformation vector construct used in this experiment comprised the aroA expression cassette described above, another cassette encoding a gene of interest, and flanking T-DNA borders. This expression cassette did not comprise the uidA gene. After Agrobacterium inoculation and co-culture, the corn explants were placed onto bud induction medium and cultured at 35° C. for one week followed by bud induction at 28° C. for an additional one week. The explants were then transferred to an extended bud induction medium and cultured for two weeks before being placed onto either MS regeneration medium or WPM regeneration medium. Regenerated transgenic shoots were then either grown in a Phytatray™ before being transplanted to soil or directly transplanted from petri plates to soil (direct to plug (DTP)). As shown in Table 7 the WPM regeneration medium demonstrated improved transformation as measured by NPF. As shown in Table 8 transgenic shoots grown in Phytatrays™ and regenerated in contact with WPM regeneration medium had double the rooting frequency compared to those grown in Phytatrays™ and regenerated in contact with MS regeneration medium. Transgenic shoots regenerated in contact with WPM regeneration medium also produced rooted plants sooner compared to those regenerated in contact with MS regeneration medium, as demonstrated by the increased rooted shoots at 1^{st} pull (%).

TABLE 7
WPM regeneration medium improves normal plant
frequency of transgenic corn plants.

| | | Shoot | Normal Shoot | |
|---|---|---|---|---|
| Regeneration | # | Frequency | Frequency | NPF |
| Medium | Explant | (%) | (%) | (%) |
| MS medium | 2,500 | 4.0 | 1.56 | 1.24 |
| WPM medium | 2,500 | 5.0 | 2.64 | 1.72 |
| MS medium (DTP) | 2,500 | 5.3 | N/A | 1.84 |
| WPM medium (DTP) | 2,500 | 6.6 | N/A | 2.76 |

The shoot frequency was calculated as the number of inoculated explants producing shoots divided by the total number of inoculated explants. The normal shoot frequency was calculated as the number of normal shoots divided by the total number of inoculated explants. The normal plant frequency (NPF) was calculated as the number of normal plants divided by the total number of inoculated explants.

TABLE 8
WPM regeneration media improves rooting
frequency of transgenic corn plants.

| Regeneration | Rooted Shoots | Rooted Shoots | Rooted Shoots |
|---|---|---|---|
| Medium | at 1^{st} Pull (%) | at 2^{nd} Pull (%) | Overall (%) |
| MS medium | 0 | 30 | 21 |
| WPM medium | 27 | 67 | 58 |

Rooted Shoots at 1^{st} Pull was calculated as the number of shoots with roots divided by total number of shoots at the first pull. Rooted Shoots at the 2^{nd} Pull (%) was calculated as the number of shoots with roots divided by total number of shoots at the 2^{nd} pull. Rooted Shoots Overall was calculated as the total number of shoots with roots divided by total number shoots from both the 1^{st} and 2^{nd} pulls.

A close examination of the macronutrients in MS, B5, WPM, and a low nitrogen MS regeneration media demonstrates that the WPM regeneration medium contains a low level of nitrogen and other salts compared to the other regeneration media evaluated (Table 9). The WPM regeneration medium contains about ⅕ of the total nitrogen compared to MS regeneration medium, about ½ the total salt compared to B5 regeneration medium, and about ⅓ of the total salt compared to the low nitrogen MS regeneration medium. In addition, WPM regeneration medium comprises about ⅓ of the potassium compared to MS regeneration medium and B5 regeneration medium and about ¼ of the potassium compared to low nitrogen MS regeneration medium. The results provided herein therefore demonstrate that the lower salt levels present in WPM regeneration medium likely result in the observed improved transformation and rooting frequency of transgenic corn plants regenerated in contact therewith.

TABLE 9
Macronutrient concentrations in MS, B5, WPM,
and a low nitrogen MS regeneration media.

Salt Concentration (mM)

| Salt Type | MS | B5 | WPM | Low Nitrogen MS |
|---|---|---|---|---|
| Total Nitrogen | 60.02 | 25.74 | 12.35 | 31.18 |
| NO₃ | 39.41 | 24.73 | 9.7 | 27.7 |
| NH₄ | 20.61 | 2.0 | 5.00 | 3.48 |
| SO₄ | 1.73 | 2.19 | 7.32 | 5.11 |
| PO₄ | 1.25 | 1.09 | 1.25 | 1.25 |
| K | 20.04 | 24.73 | 12.6 | 28.94 |
| Ca | 2.99 | 1.02 | 3.01 | 2.99 |
| Mg | 1.5 | 1.55 | 1.01 | 1.50 |

### Example 5

### Comparison of Different Levels of Nitrate in Regeneration Media on Corn Seed Explant Transformation

This example describes experiments and results further investigating the effect of different levels of nitrogen in the regeneration media on corn explant transformation and regeneration.

As shown in Example 4, lower salt levels in WPM regeneration medium appear to contribute to an improved transformation and rooting frequency in corn. Since the WPM salt-based regeneration medium contained significantly less total nitrogen compared to the MS salt-based regeneration medium, additional experiments were carried out to examine the effect of different levels of nitrogen components in modified MS salt-based regeneration media on rooting frequency and regeneration.

TABLE 10
Nitrogen levels in salt-based regeneration media.

| Nitrogen | 1 × | ½ × | ¼ × | WPM-like |
|---|---|---|---|---|
| Source | Nitrogen | Nitrogen | Nitrogen | Nitrogen |
| NH₄NO₃ (g/L) | 1.65 | 0.825 | 0.413 | 0.413 |
| KNO₃ (g/L) | 1.9 | 0.95 | 0.475 | — |

Four MS salt-based regeneration media containing different amounts of nitrogen-containing components were tested, along with WPM as a control. The regeneration media contained the macro and micronutrients as described in Murashige and Skoog (1962), but without ammonium nitrate and potassium nitrate in the salt mixture to allow for the amounts of these components to be added separately. Ammonium nitrate and potassium nitrate were added to the media in the amounts shown in Table 10, representing full strength (1×), half strength (½×), and quarter strength (¼×) relative to the standard amounts in a MS salt mixture, or in the same amount as in WPM salt (WPM-like). The rest of the medium ingredients were the same as in the standard MS salt mixture. The WPM was also used as a control.

In the first experiment, excised corn explants from an elite germplasm were inoculated with Agrobacterium at two optical densities (OD 0.25 or 1) as described in Example 1. The Agrobacterium harbored two expression cassettes flanked by the T-DNA borders on the Ti plasmid. The first expression cassette comprised a chloroplast targeted CP4 EPSPS gene under the control of a constitutive promoter as a selection marker for transgenic events. The second expression cassette comprised a uidA gene under the control of a different constitutive promoter as a screenable marker for visual identification of transformants. The inoculated corn explants were transferred to co-culture plates (25 mm×100 mm) containing a piece of sterile Whatman #1 filter paper (82 mm) wetted with 1.25 mL of rehydration medium without Agrobacterium (the treatments of inoculation OD 0.25), or with 1.25 mL of Agrobacterium at an OD of 1.0 (the treatments of inoculation OD 1.0). The co-culture plates were incubated at 20° C. and about 65% relative humidity with a photoperiod of 16 hours light/8 hours dark for 6 days.

After inoculation and co-culture, the inoculated explants went through the bud induction and extended bud induction (EBI) steps as described in Example 1. Following EBI, explants were transferred to different regeneration media at about 30 explants per petri plate. The plates were incubated at 28° C. with a photoperiod of 16 hours light/8 hours dark, and a light intensity target of 160 PAR. After 2-3 weeks, the responding explants were transferred to PlantCons™ with the same media and incubated for another 2-3 weeks before data was obtained.

The results of the experiment are summarized in Table 11. Rooting frequency (%) is defined as the number of rooted shoots divided by number of explants producing shoots×100. Transformation (TFN) frequency (%) is defined as the number of rooted shoots divided by the number of inoculated explants×100, which is a measurement of transformation and regeneration frequency. Average rooting frequency is the average for the specific nitrogen level, including the treatments of inoculation OD at 0.25 and 1.0. Similarly, average transformation frequency is the average for each medium. As shown in Table 11, the average rooting frequency increased as the nitrogen level decreased from 1× to ¼× for the MS salt-based regeneration media. The WPM-like regeneration medium contained a similar but lower level of nitrogen and produced a similar or slightly higher average rooting frequency. Media containing lower levels of nitrogen also improved average transformation frequency.

TABLE 11
Effect of nitrate level in regeneration medium on regeneration and transformation.

| | | | # | | | | Average | Average |
|---|---|---|---|---|---|---|---|---|
| | | # | Explant | # | Rooting | TFN | Rooting | TFN |
| Nitrogen | Inoculation | Inoculated | with | Rooted | Frequency | Frequency | Frequency | Frequency |
| Level | OD | Explant | Shoot | Shoot | (%) | (%) | (%) | (%) |
| ¼ x | 0.25 | 456 | 14 | 13 | 92.86 | 2.85 | 92.86 | 2.19 |
| | 1 | 456 | 8 | 7 | 87.5 | 1.54 | | |
| ½ x | 0.25 | 456 | 18 | 18 | 100 | 3.95 | 88.89 | 2.74 |
| | 1 | 456 | 9 | 7 | 77.78 | 1.54 | | |
| 1 x | 0.25 | 456 | 15 | 9 | 60 | 1.97 | 75 | 1.97 |
| | 1 | 456 | 10 | 9 | 90 | 1.97 | | |
| WPM- | 0.25 | 456 | 7 | 7 | 100 | 1.54 | 97.37 | 2.74 |
| Like | 1 | 456 | 19 | 18 | 94.74 | 3.95 | | |
| WPM | 0.25 | 456 | 15 | 12 | 80 | 2.63 | 82.86 | 2.63 |
| | 1 | 456 | 14 | 12 | 85.71 | 2.63 | | |

Two additional experiments (Experiments 2 and 3) were conducted following the same protocol as in the first experiment described above, except that (1) the corn explants were from a different elite corn germplasm; (2) only inoculation OD of 0.25 was used; (3) one of the experiments used Agrobacterium harboring a different construct comprising only the CP4 EPSPS expression cassette, and (4) only a subset of the responding greening explants from the extended bud induction media were randomly selected and transferred to the regeneration media in petri plates, followed by transfer to PlantCons™

The results of these two experiments are shown in Table 12. As described in the preceding paragraph, #Responding Explants to Regeneration Plates represents only a subset of the responding greening explants that were randomly picked across all explants after extended bud induction and transferred to regeneration media for further testing. Adjusted Regeneration Frequency (%) is expressed as the number of rooted shoots divided by number of responding explants transferred to regeneration plates×100. While the results of these particular experiments did not show a clear change in rooting frequency, the adjusted regeneration frequency with the ¼× nitrogen treatment appeared consistently higher in both experiments.

TABLE 12
Effect of nitrogen levels in regeneration medium on regeneration and transformation.

| | | | # Responding | | | | Adjusted |
|---|---|---|---|---|---|---|---|
| | | # | Explants to | # | # | Rooting | Regeneration |
| | Nitrogen | Explants | Regeneration | Explant | Rooted | Frequency | Frequency |
| Expt # | Level | Inoculated | Plates | with Shoot | Shoot | (%) | (%) |
| 2 | ¼ x | 456 | 104 | 44 | 37 | 84.09 | 35.58 |
| | ½ x | 456 | 115 | 35 | 31 | 88.57 | 26.96 |
| | 1 x | 456 | 110 | 37 | 32 | 86.49 | 29.09 |
| | WPM- | 456 | 110 | 34 | 31 | 91.18 | 28.18 |
| | Like | | | | | | |
| | WPM | 456 | 119 | 35 | 33 | 94.29 | 27.73 |
| 3 | ¼ x | 1000 | 126 | 47 | 37 | 78.72 | 29.37 |
| | ½ x | 1000 | 104 | 42 | 34 | 80.95 | 32.69 |
| | 1 x | 1000 | 126 | 51 | 40 | 78.43 | 31.75 |
| | WPM- | 1000 | 116 | 37 | 26 | 70.27 | 22.41 |
| | Like | | | | | | |
| | WPM | 1000 | 116 | 41 | 28 | 68.29 | 24.14 |

### Example 6

### Force-Assisted Transformation Improves Corn Explant Transformation

This example demonstrates that force-assisted transformation improves the transformation of corn embryo explants excised from dry seeds. In this example, the force-assisted transformation may comprise subjecting the explant to high pressure, centrifugation, or high pressure and centrifugation prior to or during inoculation with Agrobacterium.

Experiments were designed to evaluate the transformation of mature corn embryo explants from dry seeds subjected to centrifugation prior to or during inoculation with Agrobacterium. Explants were surface sanitized with 95% ethanol and 200 ppm active chlorine, rinsed 3 times, rehydrated in 20% PEG4000 INO medium comprising ⅖ strength of B5 macro salt, 1/10 strength of B5 micro salts and vitamins, 1 g/L potassium nitrate, 30 g/L dextrose, 3.9 g/L 2-(N-morpholino)ethanesulfonic acid (MES) and 20% PEG4000, pH 5.4 for 3 hours, rinsed, and floatation enriched prior to inoculation. The dry embryo explants were then subjected to one of the following treatments: 1) sonication for 1 min at 45 kHz followed by incubation for 30 minutes at room temperature in the presence of Agrobacterium inoculum; 2) centrifugation at 291×g for 30 minutes prior to Agrobacterium inoculation followed by sonication for 1 min at 45 kHz and incubation for 30 minutes at room temperature in the presence of Agrobacterium inoculum; 3) sonication for 1 min at 45 kHz followed by centrifugation at 291×g for 30 minutes at room temperature in the presence of Agrobacterium inoculum; 4) incubation for 30 minutes at room temperature in the presence of Agrobacterium inoculum; 5) centrifugation at 291×g for 30 minutes prior to Agrobacterium inoculation followed by incubation in the presence of Agrobacterium inoculum for 30 minutes at room temperature; or 6) centrifugation at 291×g for 30 minutes at room temperature in the presence of Agrobacterium inoculum. The Agrobacterium used for inoculation in this experiment comprised a plant transformation vector comprising three expression cassettes, one first encoding β-glucuronidase (GUS), the second encoding GFP and the third encoding aadA. The uidA (GUS) gene was under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The gfp gene was under the control of enhanced 35S RNA promoter from CaMV, the 5′ untranslated leader of wheat major chlorophyll a/b-binding protein, an intron from the rice actin 1 gene, and the 3′ UTR of the wheat low molecular weight heat shock protein gene. The aadA gene was under the control of enhanced 35S RNA promoter, the 5′ untranslated leader of wheat major chlorophyll a/b-binding protein fused with the first intron from rice Actin 1, and the 3′ UTR of the nopaline synthase gene of Agrobacterium tumefaciens Ti plasmid. Centrifugation was performed using a Sorvall™ RC3BP centrifuge and a H6000A rotor (Thermo Fisher Scientific, Waltham, MA, USA).

Following inoculation, the explants were co-cultured for 5 days at 23° C. and 70% relative humidity with a photoperiod of 16 hours light/8 hours dark in 1.25 ml INO media comprising ⅖ strength, 1/10 strength of B5 micro salts and vitamins, 1 g/L potassium nitrate, 30 g/L dextrose, 3.9 g/L 2-(N-morpholino)ethanesulfonic acid (MES), pH 5.4, plus 50 ppm nystatin, 10 ppm thiabendazole (TBZ), and 50 ppm_{pentachloronitrobenzene} (PCNB). Explants were then sampled and analyzed for GUS activity using a quantitative MUG assay. As shown in Table 13, explants centrifuged either prior to or during inoculation demonstrated a significant increase in overall transient GUS expression. In this experiment, sonication did not have a statistically significant impact on transient GUS expression. There was also no statistically significant difference in transient GUS expression observed between explants centrifuged prior to Agrobacterium inoculation and those centrifuged during Agrobacterium inoculation. In these experiments, a negative GUS expression measurement is interpreted as no GUS staining.

TABLE 13
Improved Transient Expression in Explants Centrifuged
either Prior to or During Inoculation.

| | | Mean GUS | |
|---|---|---|---|
| | # | Expression | Standard |
| Treatment | Replication | (pmol/μg protein/hr) | Deviation |
| No centrifugation | 24 | −5.095 | 1.388 |
| Centrifugation at 291 × g | 24 | 0.837 | 2.282 |
| during inoculation | | | |
| Centrifugation at 291 × g | 24 | 0.903 | 2.199 |
| prior to inoculation | | | |

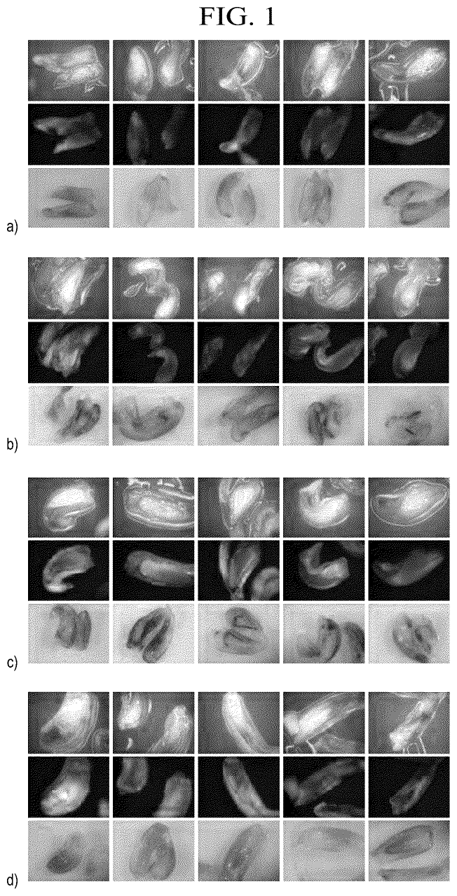

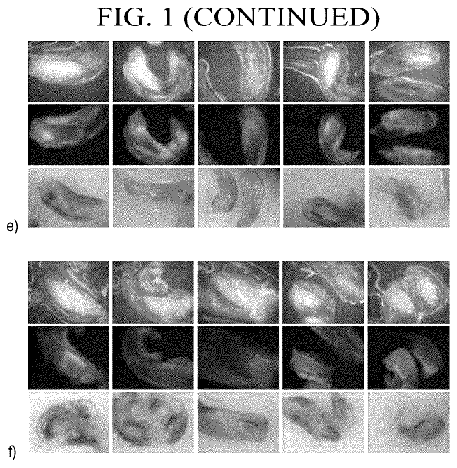

In addition, five explants from each treatment group were bisected and imaged for GFP and GUS expression. As shown in FIG. 1, transient expression was higher in the coleoptile and leaf base of explants centrifuged either before or during inoculation. FIG. 1 shows brightfield images, fluorescent images, and X-gal staining of explants subjected to: a) sonication for 1 min at 45 kHz followed by incubation for 30 minutes at room temperature in the presence of Agrobacterium inoculum; b) centrifugation at 291×g for 30 minutes prior to Agrobacterium inoculation followed by sonication for 1 min at 45 kHz and incubation for 30 minutes at room temperature in the presence of Agrobacterium inoculum; c) sonication for 1 min at 45 kHz followed by centrifugation at 291×g for 30 minutes at room temperature in the presence of Agrobacterium inoculum; d) incubation for 30 minutes at room temperature in the presence of Agrobacterium inoculum; e) centrifugation at 291×g for 30 minutes prior to Agrobacterium inoculation followed by incubation in the presence of Agrobacterium inoculum for 30 minutes at room temperature; or f) centrifugation at 291×g for 30 minutes at room temperature in the presence of Agrobacterium inoculum.

Experiments were designed to evaluate the transformation of mature corn embryo explants excised from dry seed subjected to centrifugation during Agrobacterium inoculation at 4° C. or 23° C. Explants were surface sanitized with 95% ethanol and 200 ppm active chlorine, rinsed 3 times, rehydrated in 20% PEG4000 in INO medium comprising ⅖ strength of B5 macro salts, 1/10 strength of B5 micro salts and vitamins, 1 g/L potassium nitrate, 30 g/L dextrose, 3.9 g/L 2-(N-morpholino)ethanesulfonic acid (MES), 20% PEG4000, pH 5.4 for 3 hours, rinsed, and floatation enriched prior to inoculation. The dry embryo explants were then subjected to one of the following treatments: 1) sonication for 1 min at 45 kHz followed by incubation for 30 minutes at 23° C. in the presence of Agrobacterium inoculum; 2) sonication for 1 min at 45 kHz followed by incubation for 30 minutes at 4° C. in the presence of Agrobacterium inoculum; 3) sonication for 1 min at 45 kHz followed by centrifugation at 291×g for 30 minutes at 23° C. in the presence of Agrobacterium inoculum; 4) sonication for 1 min at 45 kHz followed by centrifugation at 291×g for 30 minutes at 4° C. in the presence of Agrobacterium inoculum; 5) incubation for 30 minutes at 23° C. in the presence of Agrobacterium inoculum; 6) incubation for 30 minutes at 4° C. in the presence of Agrobacterium inoculum; 7) centrifugation at 291×g for 30 minutes at 23° C. in the presence of Agrobacterium inoculum; or 8) centrifugation at 291×g for 30 minutes at 4° C. in the presence of Agrobacterium inoculum. The Agrobacterium used for inoculation in this experiment comprised a plant transformation vector comprising three expression cassettes, one first encoding β-glucuronidase (GUS), the second encoding GFP, and the third encoding aadA. The uidA (GUS) gene was under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The gfp gene was under the control of enhanced 35S RNA promoter from CaMV, the 5′ untranslated leader of wheat major chlorophyll a/b-binding protein, an intron from the rice actin 1 gene, and the 3′ UTR of the wheat low molecular weight heat shock protein gene. The aadA gene was under the control of enhanced 35S RNA promoter, the 5′ untranslated leader of wheat major chlorophyll a/b-binding protein fused with the first intron from rice Actin 1, and the 3′ UTR of the nopaline synthase gene of Agrobacterium tumefaciens Ti plasmid. Centrifugation was performed using a Sorvall™ RC3BP centrifuge and a H6000A rotor (Thermo Fisher Scientific, Waltham, MA, USA).

Following inoculation, the explants were co-cultured for 6 days at 23° C. and 70% relative humidity with a photoperiod of 16 hours light/8 hours dark in 1.25 ml INO media comprising ⅖ strength, 1/10 strength of B5 micro salts and vitamins, 1 g/L potassium nitrate, 30 g/L dextrose, 3.9 g/L 2-(N-morpholino)ethanesulfonic acid (MES), pH 5.4, plus 50 ppm nystatin, 10 ppm thiabendazole (TBZ), and 50 ppm pentachloronitrobenzene (PCNB). Explants were then analyzed for GUS activity using a quantitative MUG assay. As shown in Table 14, explants centrifuged during inoculation demonstrated a significant increase in overall transient GUS expression regardless of the temperature at which the inoculation was performed. In this experiment, neither incubation temperature nor sonication had a statistically significant impact on transient GUS expression. As for Table 30 above, a negative GUS expression measurement is interpreted as no GUS staining.

TABLE 14
Improved Transient Expression in Explants Centrifuged
During Inoculation at Various Temperatures.

| | # | Mean GUS Expression | Standard |
|---|---|---|---|
| Treatment | Replication | (pmol/μg protein/hr) | Deviation |
| No centrifugation | 12 | −0.7633 | 0.4423 |
| Centrifugation at 291 g | 12 | 0.6575 | 0.5028 |

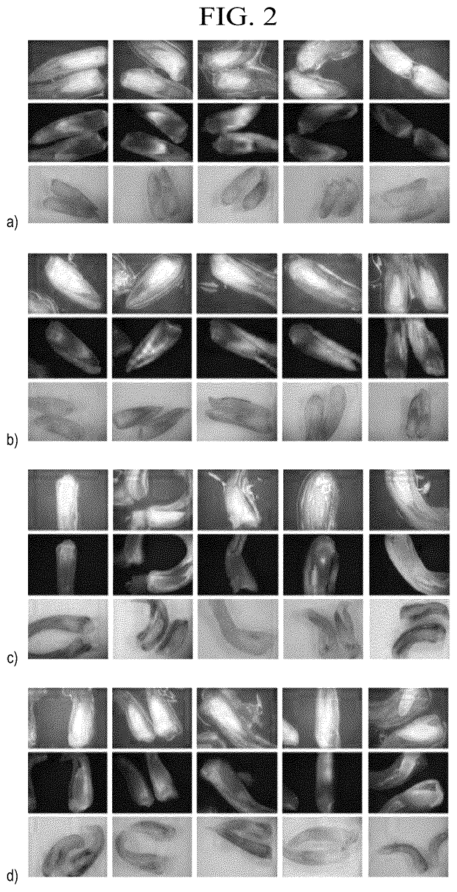

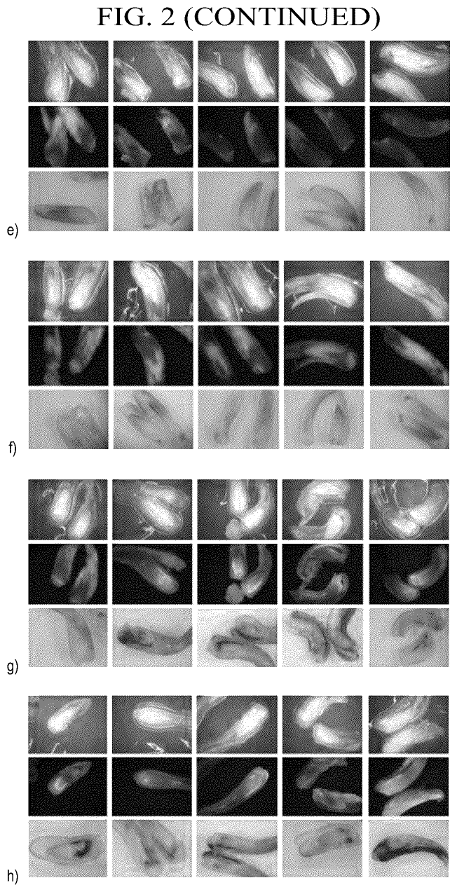

In addition, five explants from each treatment group were bisected and imaged for GFP and GUS expression. As shown in FIG. 2, transient expression was higher in the leaf base of explants centrifuged during inoculation. FIG. 2 shows brightfield images, fluorescent images, and X-gal staining of explants subjected to: a) sonication for 1 min at 45 kHz followed by incubation for 30 minutes at 23° C. in the presence of Agrobacterium inoculum; b) sonication for 1 min at 45 kHz followed by incubation for 30 minutes at 4° C. in the presence of Agrobacterium inoculum; c) sonication for 1 min at 45 kHz followed by centrifugation at 291×g for 30 minutes at 23° C. in the presence of Agrobacterium inoculum; d) sonication for 1 min at 45 kHz followed by centrifugation at 291×g for 30 minutes at 4° C. in the presence of Agrobacterium inoculum; e) incubation for 30 minutes at 23° C. in the presence of Agrobacterium inoculum; f) incubation for 30 minutes at 4° C. in the presence of Agrobacterium inoculum; g) centrifugation at 291×g for 30 minutes at 23° C. in the presence of Agrobacterium inoculum; or h) centrifugation at 291×g for 30 minutes at 4° C. in the presence of Agrobacterium inoculum.

Experiments were designed to evaluate transformation of mature corn embryo explants excised from dry seeds exposed to centrifugation or high pressure prior to inoculation with Agrobacterium. Explants were sanitized sequentially with 70% ethanol for 5 minutes and 10% Clorox®, rinsed 3 times, and rehydrated in inoculation medium comprising ⅖ strength of B5 macro salts, 1/10 strength of B5 micro salts and vitamins, 1 g/L potassium nitrate, 30 g/L dextrose, 3.9 g/L 2-(N-morpholino)ethanesulfonic acid (MES), pH 5.4, and 30 ppm Clearys® fungicide for 1 hour prior to the exposure to high pressure or centrifugation. Following exposure to high pressure or centrifugation, the dry embryo explants were inoculated with Agrobacterium comprising a plant transformation vector comprising two expression cassettes. One cassette comprises the uidA gene, which encodes β-glucuronidase (GUS), under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The other cassette comprises the aroA gene, which encodes class II EPSPS enzyme (5-enolpyruvylshikimate-3-phosphate synthase) and is targeted to the chloroplast by the transit peptide of Arabidopsis EPSPS, under the control of rice actin 1 promoter, leader and intron, and the 3′ UTR of the nopaline synthase gene of Agrobacterium tumefaciens Ti plasmid, which functions to direct polyadenylation of the mRNA, for 1 hour at room temperature at various time points following exposure. Centrifugation was performed by placing explants in 50 ml polypropylene centrifuge tubes (Falcon® Bluemax™, Becton Dickinson 4-2098-11) with about 50 ml inoculation medium, as described above, and centrifuging using a Sorvall™ RC3BP centrifuge and a H6000A rotor (Thermo Fisher Scientific, Waltham, MA, USA). High pressure was applied by loading dry embryo explants in 20 ml of the inoculation medium described above into a French Press 40K pressure cell (Thermo® IEC, FA-032) and exposing the explants to 200 psig under high ratio for 5 minutes, which corresponds to a pressure of 3334 psi (227 atm) within the pressure cell.

Following inoculation, the explants were subjected to co-culture, bud induction, extended bud induction, and regeneration. Briefly, the explants were co-cultured with Agrobacterium for 5 days by plating on filter paper in a PlantCon™ (MP Biomedicals, LLC catalog #26-720-02) growth chamber with 1.25 ml inoculation medium as described above comprising 30 ppm Clearys® and 5 ppm 2,4-D at 23° C. with a photoperiod of 16 hours light/8 hours dark. Co-cultured explants were transferred to solid bud induction medium comprising MS salts, B5 vitamins, 30 g/L sucrose, 0.69 g/L proline, 1 g/L NZ amine-A (casein enzymatic hydrolysate; Millipore Sigma), 2 mg/L glycine, 1 g/L MES, 1 mg/L 2,4-dichlorophenoxyacetic acid (2,4-D), 10 mg/L 6-benzylaminopurine (BAP), 400 mg/L carbenicillin, 200 mg/L cefotaxime, 100 mg/L timentin, and 3.5 g/L low EEO agarose, pH 5.8, and incubated for two weeks at 28° C. with a photoperiod of 16 hours light/8 hours of dark. Explants were then transferred to filter paper sandwiches for liquid selection at 28° C. with a photoperiod of 16 hours light/8 hours dark. During liquid selection, 10 ml of selection media comprising MS salts, B5 vitamins, 30 g/L sucrose, 0.69 g/L proline, 2 mg/L glycine, 1 g/L MES, 400 mg/L carbenicillin, 200 mg/L cefotaxime, 100 mg/L timentin, and 20 μM glyphosate, pH 5.8 was initially added to each plate and an additional 5 ml was added weekly. Shoots were transferred to bud induction and selection media comprising MS salts, B5 vitamins, 30 g/L sucrose, 0.69 g/L proline, 2 mg/L glycine, 1 g/L MES, 1 mg/L 2,4-dichlorophenoxyacetic acid (2,4-D), 10 mg/L 6-benzylaminopurine (BAP), 400 mg/L carbenicillin, 200 mg/L cefotaxime, 100 mg/L timentin, 20 μM glyphosate, and 3.5 g/L low EEO agarose, pH 5.8 8 to 10 weeks post-inoculation. Rooted R₀ events were sent to greenhouse for phenotyping and copy number analysis.

As shown in Table 15, exposure to either high pressure or centrifugation prior to inoculation with Agrobacterium significantly increased the transformation frequency of corn dry embryo explants. Furthermore, transformation frequency declined as the elapsed time between exposure to high pressure or centrifugation and inoculation increased. Transformation frequency was calculated as the number of R₀ plants divided by the total number of explants transferred to bud induction media.

TABLE 15
Improved Transformation Frequency of Corn Explants Exposed
to Centrifugation or High Pressure prior to Transformation.

Force
Treatment:

| Time post- | | | Time post- | | Estimated | Initial | Explants | | |
|---|---|---|---|---|---|---|---|---|---|
| Treatment | Force | | Treatment | | Treatment | Explants | used in | R₀ | Transformation |
| inoculated | Treatment | | inoculated | | Force | inoculated | stables | plants | Frequency (%) |
| 30 min 1 g: | 1 | g | 15 | min | 0.5N | 1522 | 1387 | 0 | 0.0% |
| 15 min | | | | | | | | | |
| 30 min 1 g: | 1 | g | 30 | min | 0.5N | 1522 | 1387 | 0 | 0.0% |
| 30 min | | | | | | | | | |
| 30 min 1 g: | 1 | g | 6 | hr | 0.5N | 1529 | 1394 | 0 | 0.0% |
| 6 hr | | | | | | | | | |
| 30 min 1 g: | 1 | g | 30 | hr | 0.5N | 1507 | 1372 | 0 | 0.0% |
| 30 hr | | | | | | | | | |
| 30 min 654 g: | 654 | g | 15 | min | 320N | 1532 | 1397 | 45 | 3.2% |
| 15 min | | | | | | | | | |
| 30 min 654 g: | 654 | g | 30 | min | 320N | 1510 | 1375 | 34 | 2.5% |
| 30 min | | | | | | | | | |
| 30 min 654 g: | 654 | g | 6 | hr | 320N | 1526 | 1391 | 38 | 2.7% |
| 6 hr | | | | | | | | | |
| 30 min 654 g: | 654 | g | 30 | hr | 320N | 1601 | 1466 | 36 | 2.5% |
| 30 hr | | | | | | | | | |
| 30 min 2619 g: | 2619 | g | 15 | min | 1280N | 1551 | 1416 | 37 | 2.6% |
| 15 min | | | | | | | | | |
| 30 min 2619 g: | 2619 | g | 30 | min | 1280N | 1541 | 1406 | 49 | 3.5% |
| 30 min | | | | | | | | | |
| 30 min 2619 g: | 2619 | g | 6 | hr | 1280N | 1535 | 1400 | 52 | 3.7% |
| 6 hr | | | | | | | | | |
| 30 min 2619 g: | 2619 | g | 30 | hr | 1280N | 1538 | 1403 | 31 | 2.2% |
| 30 hr | | | | | | | | | |
| 5 min 227 atm: | 227 | atm | 15 | min | 13000N | 1598 | 1463 | 54 | 3.7% |
| 15 min | | | | | | | | | |
| 5 min 227 atm: | 227 | atm | 30 | min | 13000N | 1519 | 1384 | 37 | 2.7% |
| 30 min | | | | | | | | | |
| 5 min 227 atm: | 227 | atm | 6 | hr | 13000N | 1573 | 1438 | 33 | 2.3% |
| 6 hr | | | | | | | | | |
| 5 min 227 atm: | 227 | atm | 30 | hr | 13000N | 1776 | 1641 | 17 | 1.0% |
| 30 hr | | | | | | | | | |

Experiments were designed to evaluate the transformation of mature corn embryo explants exposed to high pressure prior to or during inoculation with Agrobacterium. In this experiment, corn dry embryo explants were not surface sanitized. High pressure was applied by loading dry embryo explants in either 1) 20 ml of inoculation medium comprising ⅖ strength of B5 macro salts, 1/10 of B5 micro salts and vitamins, 1 g/L potassium nitrate, 30 g/L dextrose and 3.9 g/L MES; or 2) 20 ml of Agrobacterium inoculum in inoculation medium into a French Press 40K pressure cell (Thermo® IEC, FA-032) and exposing the explants to a pressure ranging from 14.7 psi to 30,000 psi. Dry embryo explants were then inoculated for 30 minutes with Agrobacterium comprising a plant transformation vector comprising two expression cassettes. One cassette comprises the uidA gene, which encodes β-glucuronidase (GUS), under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The other cassette comprises the aroA gene, which encodes class II EPSPS enzyme (5-enolpyruvylshikimate-3-phosphate synthase) and is targeted to the chloroplast by the transit peptide of Arabidopsis EPSPS, under the control of the rice actin 1 promoter, leader and intron, and the 3′ UTR of the nopaline synthase gene of Agrobacterium tumefaciens Ti plasmid which functions to direct polyadenylation of the mRNA.

Following inoculation, the explants were subjected to co-culture, bud induction, extended bud induction, and regeneration. Briefly, the explants were co-cultured with Agrobacterium for 5 days by plating on filter paper in a PlantCon™ (MP Biomedicals, LLC catalog #26-720-02) growth chamber with 1.25 ml inoculation medium as described above comprising 30 ppm Clearys® and 5 ppm 2,4-D at 23° C. with a photoperiod of 16 hours light/8 hours dark. Co-cultured explants were transferred to solid bud induction medium comprising MS salts, B5 vitamins, 30 g/L sucrose, 0.69 g/L proline, 1 g/L NZ amine-A (casein enzymatic hydrolysate; Millipore Sigma), 2 mg/L glycine, 1 g/L MES, 1 mg/L 2,4-dichlorophenoxyacetic acid (2,4-D), 10 mg/L 6-benzylaminopurine (BAP), 400 mg/L carbenicillin, 200 mg/L cefotaxime, 100 mg/L timentin, and 3.5 g/L low EEO agarose, pH 5.8, and incubated for two weeks at 28° C. with a photoperiod of 16 hours light/8 hours of dark. Explants were then transferred to filter paper sandwiches for liquid selection at 28° C. with a photoperiod of 16 hours light/8 hours dark. During liquid selection, 10 ml of selection media comprising MS salts, B5 vitamins, 30 g/L sucrose, 0.69 g/L proline, 2 mg/L glycine, 1 g/L MES, 400 mg/L carbenicillin, 200 mg/L cefotaxime, 100 mg/L timentin, and 20 μM glyphosate, pH 5.8 was initially added to each plate and an additional 5 ml was added weekly. Shoots were transferred to bud induction and selection media comprising MS salts, B5 vitamins, 30 g/L sucrose, 0.69 g/L proline, 2 mg/L glycine, 1 g/L MES, 1 mg/L 2,4-dichlorophenoxyacetic acid (2,4-D), 10 mg/L 6-benzylaminopurine (BAP), 400 mg/L carbenicillin, 200 mg/L cefotaxime, 100 mg/L timentin, 20 μM glyphosate, and 3.5 g/L low EEO agarose, pH 5.8 8 to 10 weeks post-inoculation. Rooted R₀ events were sent to greenhouse for phenotyping and copy number analysis. As shown, in Table 16 transformation frequency was significantly increased in explants exposed to 1667 psi, 3334 psi, 10,000 psi, and 20,000 psi of pressure. In this experiment, exposure to 30,000 psi appeared to be lethal. Transformation frequency was calculated as the number of R₀ plants divided by the total number of explants transferred to bud induction media.

TABLE 16
Improved Transient Expression in Explants Exposed
to High Pressure Prior to or During Inoculation.

| | Number Explants | | Transformation |
|---|---|---|---|
| Treatment | used in Stables | R₀ plants | Frequency (%) |
| Inoculation medium, | 117 | 0 | 0.0% |
| 14.7 psi | | | |
| Inoculation medium, | 25 | 2 | 8.0% |
| 1,667 psi | | | |
| Inoculation medium, | 37 | 3 | 8.1% |
| 3,334 psi | | | |
| Agrobacterium | 70 | 4 | 5.7% |
| inoculum, 1,667 psi | | | |
| Agrobacterium | 51 | 4 | 7.8% |
| inoculum, 3,334 psi | | | |
| Inoculation medium, | 58 | 6 | 10.3% |
| 10,000 psi | | | |

Experiments were designed to evaluate the transformation frequency of mature corn embryo explants from dry seeds subjected to centrifugation, high pressure, or a combination of centrifugation and high-pressure during Agrobacterium inoculation. Two batches of explants were prepared as described in Example 1. Each explant batch was subjected to one of the following treatments: (1) centrifugation at 2,620×g for 30 minutes in the presence of Agrobacterium inoculum; (2) high pressure at 300 psi for 3 minutes in the presence of Agrobacterium inoculum; (3) high pressure at 300 psi for 3 minutes prior to inoculation followed by centrifugation at 2,620×g for 30 minutes in the presence of Agrobacterium inoculum; or (4) high pressure at 300 psi for 3 minutes in the presence of Agrobacterium inoculum followed by centrifugation at 2,620×g for 30 minutes in the presence of Agrobacterium inoculum. The third treatment, high pressure prior to inoculation followed by centrifugation in the presence of Agrobacterium inoculum, allowed for the contribution of the high-pressure component in the combined steps of the fourth treatment group to be evaluated. Each of the two explant batches were divided into one of the four treatment groups. Three repetitions were completed for each treatment using 1,140 explants per repetition. High pressure was applied using a Model 600-EXP Pressure Chamber (PMS Instrument Company, Albany, OR, USA), which was connected to a larger external pressure chamber through the external port. Nitrogen gas was used to pressurize the chamber. Centrifugation was applied using a Sorvall™ RC3BP centrifuge (Thermo Fisher Scientific, Waltham, MA, USA).

The corn dry embryo explants were sterilized with a solution of polyethylene glycol (PEG) and ethanol for 4 minutes and then rehydrated with 40 ml of rehydration medium for two hours. Explants were then inoculated with Agrobacterium while being exposed to centrifugation, high pressure, or a combination of centrifugation and high pressure as described above. The Agrobacterium used for inoculation comprised a binary plant transformation vector with two expression cassettes, the first encoding β-Glucuronidase (GUS) and the second encoding an EPSPS-CP4. The initial rehydration and subsequent high pressure and centrifugation treatments were performed using 50 ml Corning® mini bioreactor tubes with vented caps (catalog number CLS431720, Sigma-Aldrich, St. Louis, MO, USA).

Following inoculation, the explants were subjected to co-culture, bud induction, extended bud induction, and regeneration as described herein. Briefly, the explants were co-cultured for 5 days in a Percival® growth chamber at 20° C. and 70% relative humidity with a photoperiod of 16 hours light/8 hours dark. Each co-culture tray holds up to approximately 570 explants, therefore two trays (or up to about 1,140 explants) were used per treatment repetition. Co-cultured explants from each tray were then transferred to 5 trays containing bud induction medium and incubated for one week at 33° C. and 70% relative humidity with a photoperiod of 16 hours light/8 hours of dark. Explants from each bud induction tray were then transferred to two trays of extended bud induction medium with 25 μM glyphosate for selection and incubated for one week at 33° C. and 70% relative humidity with a photoperiod of 16 hours light/8 hours of dark. Explants were transferred to regeneration medium in Vivi® trays (VIVI Green Innovators, ś-Gravendeel, The Netherlands) with 20 μM glyphosate selection and incubated for 4 to 6 weeks to allow transgenic plants to develop. Following regeneration, phenotypically normal plants were plugged into plugging medium (Product #72-R, Gro-Tech.com Falmouth, ME, USA) and incubated in trays (Item #PL-O36-STAR—HW-VH-BK-50, Clearwater, MN, USA). Leaf tissue samples for molecular assays were obtained from each plugged plant. Plugged plants were then moved to a growth chamber for hardening.

Based on molecular data, transformed plants were advanced to the greenhouse for observation. GUS expression was used as a visible marker to detect transient expression following co-culture and to detect stable expression at other time points, such as during bud induction and R₀ shoot development. GUS expression assays of R₀ leaf tissue and visual observation of plants at plugging was also used to identify chimeric plants. Chimeric plants typically had the phenotype of striped leaves with green and white sectors during plugging. Molecular assays were performed on R₀ leaf tissue using real-time PCR to determine the presence and copy number of the CP4 EPSPS and uidA genes. Based on these assays, the percentage of non-chimeric transformed plants (a.k.a. normal transgenic plants or normal plants) for each treatment was determined by dividing the number of non-chimeric, and both CP4 EPSPS and uidA positive transgenic plants by the total number of plants sampled after plugging. Table 17 shows the plugging frequency, the frequency of low copy number plants (i.e., the percentage of non-chimeric plants that were positive for a low copy number of both the EPSPS CP4 gene and the uidA gene), and frequency of transformants with low copy number transgenes. The plugging frequency was calculated as the number of transgenic R₀ plants plugged divided by the total number of inoculated explants. The frequency of low copy number plants was calculated as the number of low copy number transgene-positive (i.e., plants with only one or two copies of each transgene) and non-chimeric plants divided by the total number of R₀ plants plugged. The frequency of transformants with low copy number transgene was calculated as the number of transformants with low copy number transgenes divided by the total number of inoculated explants. Each of these frequency calculations was further multiplied by 100% to express the frequency as a percentage. The calculations were made based on samples taken from a random number of plants after plugging.

TABLE 17
Improved Transformation Frequency in Corn Explants Exposed
to a Combination of High Pressure and Centrifugation.

| | | | Frequency of |
|---|---|---|---|
| | | Frequency of | Transformants |
| | Plugging | Low Copy | with Low Copy |
| Treatment | Frequency | Number Plants | Number Transgene |
| Treatment 1 | 2.2% | 53.0% | 1.2% |
| Centrifugation | | | |
| Treatment 2 | 3.0% | 53.4% | 1.6% |
| (High Pressure) | | | |
| Treatment 3 | 1.9% | 53.8% | 1.0% |
| (High Pressure, | | | |
| no Agro + | | | |
| Centrifugation) | | | |
| Treatment 4 | 3.0% | 65.6% | 2.0% |
| (High pressure + | | | |
| Centrifugation) | | | |

As shown in Table 17, the frequency of low copy number plants and frequency of transformants with low copy transgene were highest when inoculation was performed using high pressure at 300 psi for 3 minutes and centrifugation at 2,620×g for 30 minutes in the presence of Agrobacterium inoculum. The low copy transformation frequency was second highest when inoculation was performed using high pressure at 300 psi for 3 minutes in the presence of Agrobacterium inoculum. Therefore, the combination of centrifugation and high-pressure during inoculation demonstrates an additional increase in transformation and low copy number frequency compared to inoculation using either high pressure or centrifugation alone.

### Example 7

### Force-Assisted Transformation Improves Wheat Explant Transformation

This example describes improvement in the transformation of wheat embryo explants excised from dry seeds using force-assisted transformation. In this example, the force-assisted transformation may comprise subjecting the explant to centrifugation during inoculation with Agrobacterium.

Explants were surface sanitized with 70% ethanol for 1 minute, rinsed, floatation enriched, rehydrated in 20% PEG4000 for 1.5 hours, and rinsed prior to inoculation. The dry embryo explants were subjected to one of the following treatments: 1) incubation for 30 minutes at 4° C. in the presence of Agrobacterium inoculum; 2) centrifugation at 72×g for 30 minutes at 4° C. in the presence of Agrobacterium inoculum; 3) centrifugation at 291×g for 30 minutes at 4° C. in the presence of Agrobacterium inoculum; or 4) centrifugation at 654×g for 30 minutes at 4° C. in the presence of Agrobacterium inoculum. The Agrobacterium used for inoculation in this experiment comprised a plant transformation vector comprising three expression cassettes, one first encoding β-glucuronidase (GUS), the second encoding GFP and the third encoding aadA. The uidA (GUS) gene was under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The gfp gene was under the control of enhanced 35S RNA promoter from CaMV, the 5′ untranslated leader of wheat major chlorophyll a/b-binding protein, an intron from the rice actin 1 gene, and the 3′ UTR of the wheat low molecular weight heat shock protein gene. The aadA gene was under the control of enhanced 35S RNA promoter, the 5′ untranslated leader of wheat major chlorophyll a/b-binding protein fused with the first intron from rice Actin 1, and the 3′ UTR of the nopaline synthase gene of Agrobacterium tumefaciens Ti plasmid. Centrifugation was performed using a Sorvall™ RC3BP centrifuge and a H6000A rotor (Thermo Fisher Scientific, Waltham, MA, USA).

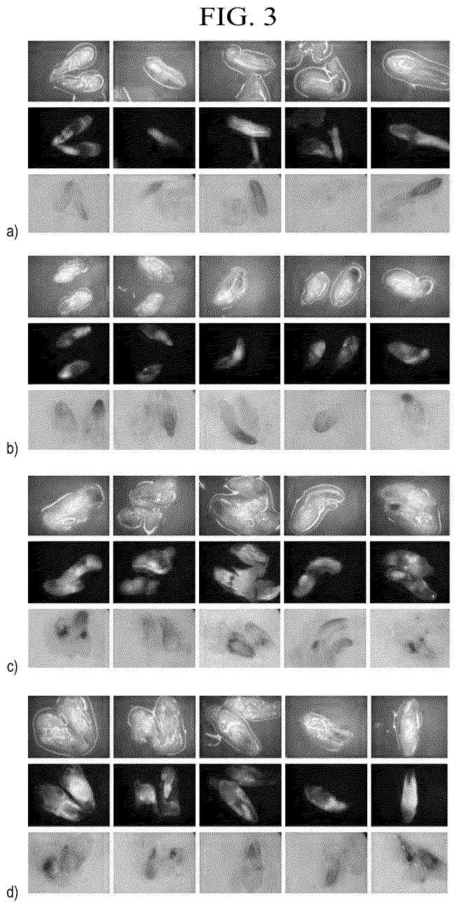

Following inoculation, the explants were co-cultured for 4 days at 23° C. and 70% relative humidity with a photoperiod of 16 hours light/8 hours dark in either 1.0 ml or 1.25 ml INO media comprising 50 ppm nystatin, 10 ppm thiabendazole (TBZ), and 50 ppm pentachloronitrobenzene (PCNB). Five explants from each treatment group were bisected and imaged for GFP and GUS expression a. As shown in FIG. 3, transient expression was increased in explants centrifuged at greater than 72×g during inoculation. FIG. 3 shows brightfield images, fluorescent images, and X-gal staining of explants subjected to: a) incubation for 30 minutes at 4° C. in the presence of Agrobacterium inoculum; b) centrifugation at 72×g for 30 minutes at 4° C. in the presence of Agrobacterium inoculum; c) centrifugation at 291×g for 30 minutes at 4° C. in the presence of Agrobacterium inoculum; or d) centrifugation at 654×g for 30 minutes at 4° C. in the presence of Agrobacterium inoculum.

### Example 8

### Effect of Gravitational Force and Centrifugation Container on Transformation of Corn Explants

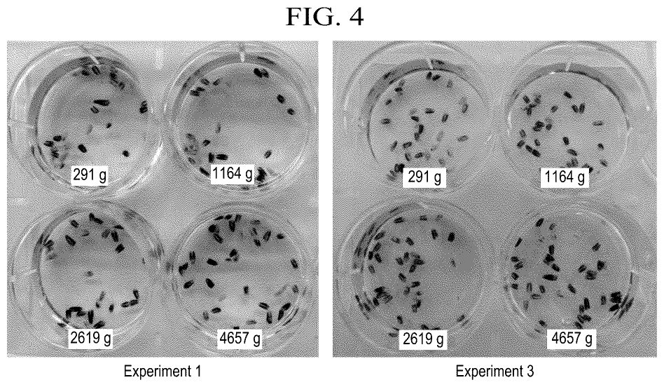

A gravitational force of 291×g or 654×g has been used to produce transgenic corn plants. Multiple experiments (Experiments 1, 2 and 3) were performed to determine if gravitational force during inoculation has a positive effect on transformation frequency (TF). As shown in FIG. 4 for two experiments (Experiments 1 and 3), transient expression was increased as gravitational force was increased from 291×g (or 1000 rpm in this experiment) to 4657×g (or 4000 rpm in this experiment). In addition, final transformation frequency was increased when gravitational force was increased from 291×g to 2619×g (or 3000 rpm in this experiment) but decreased somewhat as gravitational force was further increased to 4657×g probably due to the impact of the gravitational force on tissue culture response (see Table 18).

TABLE 18
Effect of gravitational force during
inoculation on stable transformation.

| Gravitational | Putative TF % | | |
|---|---|---|---|
| force (g) | Exp 1 | Exp 2 | Exp 3 |
| 291 | 0.13 | 0.08 | 0.4 |
| 1164 | 0.24 | 0.21 | 0.8 |
| 2619 | 0.32 | 0.37 | 2.44 |
| 4657 | 0.29 | 0.29 | 2.15 |

Falcon centrifuge tubes (50 ml) have been used for centrifugation during corn transformation, but Corning conical bottles (500 ml) have also been used to grow Agrobacterium in place of 50 ml Falcon centrifugation tubes with scale-up production implementation in mind. However, larger centrifugation containers may not be necessary with improved transformation frequencies. Multiple experiments (Experiments 1, 2, 3 and 4) were performed to investigate the effect of the centrifugation container on transformation frequency (TF). As shown in Table 19, greater TF was achieved with 50 ml Falcon tubes compared to 500 ml Corning conical bottles.

TABLE 19
Effect of centrifugation container on stable transformation.

| | |
|---|---|
| Centrifugation | Putative TF % |

| Container | Exp 1 | Exp 2 | Exp 3 | Exp 4 |
|---|---|---|---|---|
| 50 ml Falcon tube | 0.2 | 0.58 | 2.12 | 4.34 |
| 250 ml Corning | 0.06 | 0.26 | 0.36 | 0.58 |
| bottle | | | | |

### Example 9

### Rehydration of Corn Explants Prior to Agrobacterium Inoculation Improved Corn Explant Transformation

Two experiments were conducted to evaluate the effect of corn explant rehydration prior to Agrobacterium inoculation on corn explant transformation. Transformation was generally performed as described in Example 1, unless otherwise noted. Explants in each experiment were rehydrated for 0, 1, or 2 hours and each treatment group contained 3,333 corn explants.

Briefly, explants were surface sterilized using 70% ethanol for 4 min and then rinsed 3 times with sterile water prior to rehydration in medium containing ⅖ strength of B5 macro salts except for CaCl₂, which is ½ strength, 1/10 strength of B5 micro salts and vitamins, 1 g/L potassium nitrate, 30 g/L dextrose, 2.8 mg/L sequestrene, 3.9 g/L MES, 0.03 g/L Clearys 3336 WP, pH 5.4 and 5 mg/L 2,4-D for 0, 1, or 2 hours. In this experiment, the same medium used for rehydration was also used for Agrobacterium inoculation and co-culture. Explants were inoculated with Agrobacterium at an OD₆₀₀ of 0.35 with centrifugation at 600×g at 4° C. for 30 min. The Agrobacterium comprised two expression cassettes flanked by T-DNA borders on the Ti plasmid. The first expression cassette comprised a chloroplast targeted CP4 EPSPS gene under the control of a constitutive promoter. The second expression cassette comprised a uidA gene under the control of a different constitutive promoter. Following co-culture, explants were cultured either in a warm room with a photoperiod cycle of 22 hours light/2 hours dark (Photoperiod 1) or in a warm room with a photoperiod cycle of 16 hours light/8 hours dark (Photoperiod 2).

As shown in Table 20, incorporation of rehydration step consistently improved shoot frequency and transformation frequency following culture with both photoperiods. Furthermore, rehydration for 2 hours improved shoot frequency and transformation frequency as compared to rehydration for 1 hour.

TABLE 20
Rehydration improves corn seed explant shoot
frequency and transformation frequency.

| | Rehydration | | # Shoots to | Shoot | | |
|---|---|---|---|---|---|---|
| | Duration | # Corn | Rooting | Frequency | # Plants | TF |
| Photoperiod | (hr) | Explants | Medium | (%) | Plugged | (%) |
| Photoperiod 1 | 2 | 3,333 | 101 | 3.03 | 51 | 1.5 |
| Photoperiod 1 | 1 | 3,333 | 35 | 1.05 | 13 | 0.4 |
| Photoperiod 1 | 0 | 3,333 | 3 | 0.09 | 1 | 0.0 |
| Photoperiod 2 | 2 | 3,333 | 91 | 2.73 | 75 | 2.3 |
| Photoperiod 2 | 1 | 3,333 | 30 | 0.9 | 24 | 0.7 |
| Photoperiod 2 | 0 | 3,333 | 10 | 0.3 | 9 | 0.3 |

The shoot frequency was calculated as the number of inoculated explants producing shoots divided by the total number of inoculated explants. Transformation frequency or TF (%) was calculated as the number of regenerated plants plugged divided by the total number of inoculated corn explants.

### Example 10

### Higher Concentration of Agrobacterium During Inoculation and Co-Culture Improves Corn Explant Transformation

This example describes experiments and results demonstrating the effect of varying concentrations of Agrobacterium during inoculation and the addition of Agrobacterium during co-culture on transformation of corn explants.

Three initial experiments were conducted to evaluate the effect of Agrobacterium concentration on transformation of corn explants. The experiments included various concentrations (measured by optical density) of Agrobacterium during inoculation and co-culture as shown in Table 21. Briefly, explants and Agrobacterium were prepared and inoculated as described in Example 1, except the concentration (OD₆₆₀) of Agrobacterium in the inoculation medium was varied, as shown in Table 21. In Experiments 1 and 2, the Agrobacterium comprised two expression cassettes flanked by T-DNA borders on the Ti plasmid. The first expression cassette comprised a chloroplast targeted CP4 EPSPS gene under the control of a constitutive promoter as a selection marker for transgenic events. The second expression cassette comprised a uidA gene under the control of a different constitutive promoter as a screenable marker for visual identification of transformants. In Experiment 3, the Agrobacterium comprised one expression cassette encoding CP4 EPSPS flanked by T-DNA borders on the Ti plasmid.

As shown in Table 21, inoculated corn explants were transferred to co-culture plates (25 mm×100 mm) containing a piece of sterile Whatman #1 filter paper (82 mm) wetted with 1.25 mL of rehydration medium either without Agrobacterium (Treatment 1 of Experiments 1-3) or with different OD concentrations of Agrobacterium (0.25, 0.5, or 1.0) in Experiment 1, or with an OD concentration of Agrobacterium of 1.0 in Experiments 2 and 3. The co-culture plates were incubated at 20° C. and about 65% relative humidity with a photoperiod of 16 hours light/8 hours dark for 6 days.

At the end of co-culture, explants were transferred to bud induction (BI) medium and cultured for 7 days, followed by transfer to extended bud induction (EBI) medium and culture for 14 days. The BI and EBI media and culturing conditions were the same as described in Example 1. Explants from EBI were then transferred to solid regeneration medium (as described in Example 1) in petri plates for 10 days before being moved into 9 cm Vivi® trays (Vivi®, The Netherlands) containing the same regeneration medium for shoot development and rooting. The Vivi® trays were sealed with plastic film using a hand-held sealing iron and cultured at 28° C. and ambient humidity with a photoperiod of 16 hours light/8 hours dark for 32 days. Following the regeneration step, putative green transgenic plants with visible roots and no sign of chimerism were transplanted in soil plugs (2″ diameter×3″ height (Gro-Tech™, Rough and Ready, CA 95975) for further growth and development and molecular characterization.

TABLE 21
Effect of Agrobacterium concentration during
inoculation and co-culture on plugging frequency.

| | | | Agro- | Agro- | Average |
|---|---|---|---|---|---|
| | | # | bacterium | bacterium | Plugging |
| Exper- | | Repli- | Inoculation | Co-Culture | Frequency |
| iment | Treatment | cation | OD | OD | (%) |
| 1 | 1 (control) | 4 | 0.25 | 0 | 3.72 |
| | 2 | 4 | 0.25 | 0.25 | 4.0 |
| | 3 | 4 | 0.5 | 0.5 | 4.79 |
| | 4 | 4 | 1.0 | 1.0 | 5.41 |
| 2 | 1 (control) | 4 | 0.25 | 0 | 5.01 |
| | 2 | 4 | 1.0 | 1.0 | 8.46 |
| 3 | 1 (control) | 4 | 0.25 | 0 | 2.04 |
| | 2 | 4 | 1.0 | 1.0 | 2.33 |

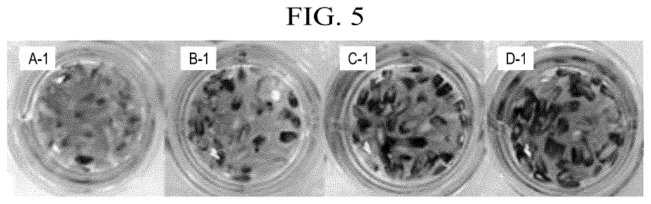

As shown in FIG. 5, transient GUS expression in Experiment 1 increased with increasing concentration of Agrobacterium. Results shown in Table 21 also demonstrate that this increase in transient expression in Experiment 1 correlates with a higher average plugging frequency. The highest plugging frequency was obtained when Agrobacterium was present at OD 1.0 during both the inoculation and co-culture steps, even though the high Agrobacterium concentration treatment appeared to slow down explant growth during bud induction and the beginning of extended bud induction when compared to the control. Similar results were obtained in Experiment 2 for both transient expression and average plugging frequency (see Table 21). In Experiment 3, a slight increase in plugging frequency (2.33%) was observed with Agrobacterium OD 1.0 in inoculation and co-culture compared to the control (2.04%) (see Table 21).

For the experiments described in this example, shoot frequency is defined as the number of shoots regenerated, including chimeric and non-chimeric shoots, divided by the total number of explants inoculated×100. Chimeric shoot frequency is defined as the number of chimeric shoots divided by the total number of shoots×100. Average shoot frequency and average chimeric shoot frequency are simply the shoot frequency and chimeric shoot frequency, respectively, averaged over multiple replicates. Plugging frequency is defined as the number of non-chimeric events that were transferred to soil divided by the total number of explants inoculated×100. Quality plug frequency is defined as the number of single copy plugged events divided by the total number of explants inoculated×100. Single copy frequency is defined as the number of single copy events divided by the total number of events generated×100.

In Experiment 2, the chimeric shoot frequency and average shoot frequency were measured and compared to evaluate whether higher Agrobacterium concentrations during inoculation and co-culture affect the frequency of chimeric shoot formation. As shown in Table 22, higher average shoot frequency was obtained with the higher Agrobacterium OD 1.0 concentration during inoculation and co-culture compared to the control, while average chimeric shoot frequency was similar between the higher OD concentration and control groups. Thus, the higher shoot frequencies produced using the higher OD concentration were not associated with increased chimerism.

TABLE 22
Effect of Agrobacterium concentration during inoculation and
co-culture on shoot frequency and chimeric shoot frequency.

| | Average Shoot | Average Chimeric Shoot |
|---|---|---|
| Treatment | Frequency (%) | Frequency (%) |
| 1 (control) | 10.32 | 56.04 |
| 2 | 18.42 | 54.51 |

To evaluate whether higher Agrobacterium concentrations during inoculation and co-culture affect the frequency of single copy events, CP4 EPSPS copy number assays were performed for events transferred to soil in Experiments 2 and 3. As shown in Table 23, % single copy events were similar between the lower standard OD and higher OD treatments.

TABLE 23
Effect of Agrobacterium concentration during inoculation
and co-culture on the frequency of single copy events.

| | Experiment | | | |
|---|---|---|---|---|
| | 2 | | 3 | |
| | Treatment | | | |
| | OD = 0.25 | OD = 1.0 | OD = 0.25 | OD = 1.0 |
| Total # Events | 112 | 180 | 422 | 539 |
| # Single Copy | 27 | 42 | 132 | 148 |
| % Single Copy | 24.0 | 23.3 | 31.3 | 27.5 |

Following the three initial experiments, several large-scale experiments were conducted, involving 16 different constructs. All 16 constructs contained the same CP4 EPSPS cassette as the selection marker, and the experiments were grouped into three groups based on the additional gene of interest expression cassette(s) carried in those constructs. Increased shoot frequency was observed with the higher Agrobacterium concentration across all experimental groups, with a statistically significant increase (denoted by “*”) for Groups 1 and 3 (see Table 24). A significant increase in plugging frequency was observed for Group 1 with the higher OD concentration, while the plugging frequency results for Groups 2 and 3 were comparable to the control. A slight increase in chimeric shoot frequency was also observed for Group 3 at the higher OD concentration. In addition, the results in Table 24 further demonstrate that transgene single copy frequency was independent of Agrobacterium concentration in these experiments. In Table 24, the underlined numbers represent uidA copy number, and the numbers without underline represent CP4 copy number. The results demonstrate that there is no significant difference in single copy frequency between the lower and higher OD concentrations. Variations measured in CP4 and uidA single copy frequencies were not statistically significant. Similarly, the quality plugging frequency was found to be comparable between the control and the higher OD treatment groups in these experiments.

TABLE 24
Effect of Agrobacterium concentration during inoculation
and co-culture; summary of large-scale experiment results.

| | | | | | | Chimeric | | | | | |
|---|---|---|---|---|---|---|---|---|---|---|---|
| | | Shoot | | Plugging | | Shoot | | | | | |
| | | Frequency | | Frequency | | Frequency | | Single Copy | | Quality Plugging | |
| | | (%) | | (%) | | (%) | | Frequency (%) | | Frequency (%) | |
| Expt | # | OD = | OD = | OD = | OD = | OD = | OD = | OD = | OD = | OD = | OD = |
| Grp | Construct | 0.25 | 1.0 | 0.25 | 1.0 | 0.25 | 1.0 | 0.25 | 1.0 | 0.25 | 1.0 |
| 1 | 8 | 1.38 | 2.19 * | 0.54 | 0.87 * | 60.91 | 60.4 | 69.2 | 64.6 | 0.37 | 0.56 |
| 2 | 1 | 3.52 | 4.08 | 1.53 | 1.67 | 56.48 | 59.14 | 39.7 | 44.7 | 0.61 | 0.75 |
| 3 | 7 | 1.81 | 2.32 * | 0.58 | 0.65 | 67.93 | 72.16* | 52.5/33.9 | 43.7/42.9 | 0.27 | 0.29 |

### Example 11

Effect of Temperature and Light Intensity during Bud Induction on Corn Explant Transformation

Two experiments were conducted to evaluate the effect of temperature and light intensity during bud induction. Explants were surface sterilized, purified, rehydrated, and inoculated with Agrobacterium as described in Example 9. Explants were then co-cultured and subjected to bud induction, extended bud induction, and regeneration as described in Example 1, unless otherwise noted.

The first experiment evaluated bud induction at two culture temperatures, 32° C. and 35° C., and two light intensities of PAR, 90 μ/m²·s and 140-150 μ/m²·s. Each treatment group contained about 2,500 corn explants. Cultures were incubated in Percival® chambers at the designated temperature and light intensity. As shown in Table 25, performing bud induction at 32° C. improved shoot frequency, normal shoot frequency, number of normal shoots to soil plug, and normal plant frequency compared to performing bud induction at 35° C. In this experiment, light intensity did not have a statistically significant effect on normal plant frequency, however, performing bud induction at 90 μ/m²·s of PAR produced a slightly higher normal plant frequency compared to performing bud induction at 140-150 μ/m²·s of PAR.

TABLE 25
Effect of temperature and light intensity during
bud induction on corn explant transformation.

| | PAR | | | | | |
|---|---|---|---|---|---|---|
| | Light | | Shoot | Normal Shoot | # Normal | |
| Temperature | Intensity | # | Frequency | Frequency | Shoot to | NPF |
| (° C.) | (μ/m² · s) | Explants | (%) | (%) | Plug | (%) |
| 32 | 90 | 2495 | 5.7 | 5.1 | 44 | 1.8 |
| 32 | 140-150 | 2500 | 6.4 | 6.0 | 41 | 1.6 |
| 35 | 90 | 2374 | 2.3 | 3.6 | 20 | 0.8 |
| 35 | 140-150 | 2500 | 0.7 | 0.6 | 8 | 0.3 |

The shoot frequency was calculated as the number of inoculated explants producing shoots divided by the total number of inoculated explants. The normal shoot frequency was calculated as the number of normal shoots divided by the total number of inoculated explants. The normal plant frequency (NPF) was calculated as the number of normal plants divided by the total number of inoculated explants.

The second experiment evaluated bud induction at four different light intensities, 90 μ/m²·s of PAR, 150 μ/m²·s of PAR, 180 μ/m²·s of PAR, and 190 μ/m²·s of PAR. Bud induction was performed at 28° C. in the 180 PAR treatment group and 33° C. in the 90 μ/m²·s of PAR, 150 μ/m²·s of PAR, and 190 μ/m²·s of PAR treatment groups. Each treatment contained 2,500 corn explants. After Agrobacterium inoculation, the explants were transferred to co-culture medium comprising ⅖ of B5 macro salts, 1/10 of B5 micro salts and vitamins, 1 g/L potassium nitrate, 30 g/L dextrose, 3.9 g/L 2-(N-morpholino)ethanesulfonic acid (MES), 0.03 g/L Clearys 3336 WP, and 5 mg/L 2,4-D, pH 5.4. The explants were then transferred to bud induction medium and cultured in a Percival® chamber, except for the 180 μ/m²·s of PAR treatment group which cultured in a Conviron® chamber, at the designated temperatures and light intensities before extended bud induction and regeneration.

As shown in Table 26, bud induction at 33° C. generally improved shoot frequency, normal shoot frequency, number of normal shoots to soil plug, and normal plant frequency, as compared to bud induction at 28° C. In addition, bud induction at 90 μ/m²·s of PAR produced the highest normal plant frequency (NPF). In this experiment there was no significant different between bud induction at 150 μ/m²·s of PAR and bud induction at 190 μ/m²·s of PAR.

TABLE 26
Effect of temperature and light intensity during
bud induction on corn explant transformation.

| | PAR | | | | | |
|---|---|---|---|---|---|---|
| | Light | | Shoot | Normal Shoot | # Normal | |
| Temperature | Intensity | # | Frequency | Frequency | Shoot to | NPF |
| (° C.) | (μ/m² · s) | Explants | (%) | (%) | Plug | (%) |
| 33 | 90 | 2,500 | 7.56 | 3.36 | 52 | 3.3 |
| 33 | 150 | 2,500 | 5.96 | 1.72 | 25 | 2.0 |
| 33 | 190 | 2,500 | 7.36 | 2.20 | 26 | 2.2 |
| 28 | 180 | 2,500 | 4 | 2.12 | 20 | 1.6 |

The shoot frequency was calculated as the number of inoculated explants producing shoots divided by the total number of inoculated explants. The normal shoot frequency was calculated as the number of normal shoots divided by the total number of inoculated explants. The normal plant frequency (NPF) was calculated as the number of normal plants divided by the total number of inoculated explants.

### Example 12

### Effect of Light Density and Temperature During Regeneration on Transformation Frequency

To test whether light density has a significant effect on transformation efficiency in a liquid culture system, an experiment was conducted to evaluate the effect of light density on mature corn embryo explant transformation during the regeneration step. In this experiment, four petri dishes of liquid culture with about 30 explants per dish were stacked and placed into plastic boxes. Half of the boxes were cultured in a first light chamber with a light density of about 60 mole/m²s and the other half were cultured in a second light chamber with a light density of about 100 mole/m²s. The results showed that the transformation frequency was greater for regeneration in the presence of a higher light density of about 100 mole/m²s compared with a lower light density of about 60 mole/m²s. In each light room, transformation frequency was greatest for the petri dishes on the top of the boxes and stacks and gradually reduced from the top layers to the bottom layers as the light density gradually decreased (Table 27). Incidentally, the temperature was also highest with plates on the top layer and gradually reduced from the top layers to the bottom layers of plates (Table 28). Thus, a combination of higher light density and plate temperature may contribute to higher transformation frequencies. The temperature outside of the sweater boxes was about 29° C. Higher light density was also shown to increase transformation frequency for both liquid and solid culture protocols, but the magnitude of the increase was greater with the liquid culture than the solid culture protocol. In addition, higher light density was shown in these experiments to increase the rooting frequency of transgenic plants (Table 29).

TABLE 27
Effect of light density on stable transformation
with liquid culture protocol.

| Light density | | | | |
|---|---|---|---|---|
| (μmole/m²s | Position of | # Visible | # Shoot | Putative |
| PPFD) | petri dish | Explants | pulled | TF % |
| 60 (top layer) | Top layer | 625 | 14 | 2.24 |
| | 2^{nd} layer | 625 | 7 | 1.12 |
| | 3^{rd} layer | 625 | 8 | 1.28 |
| | 4^{th} layer | 625 | 6 | 0.98 |
| 100 (top layer) | Top layer | 625 | 43 | 6.88 |
| | 2^{nd} layer | 625 | 28 | 4.48 |
| | 3^{rd} layer | 625 | 22 | 3.52 |
| | 4^{th} layer | 625 | 12 | 1.92 |

TABLE 28
Correlation between actual plate light
density and temperature of plate.

| Position of | Light density (μmole/m²s | Plate |
|---|---|---|
| petri dish | PPFD) | temperature (° C.) |
| Top layer | 95 | 35 |
| 2^{nd} layer | 39 | 34 |
| 3^{rd} layer | 33 | 33 |
| 4^{th} layer | 32 | 32 |

TABLE 29
Effect of light density on liquid culture versus solid culture
and the frequency of rooting of transgenic plants.

| | | Putative TF % | | Rooting frequency % | |
|---|---|---|---|---|---|
| | | 60 | 100 | 60 | 100 |
| Culture | | μmole/m²s | μmole/m²s | μmole/m²s | μmole/m²s |
| method | Exp | PPFD | PPFD | PPFD | PPFD |
| Solid | 1 | 1.6 | 2.1 | 30.4 | 57.9 |
| | 2 | 1.6 | 1.9 | 33.5 | 68.8 |
| | 3 | 3.5 | 3.7 | 37.3 | 68.5 |
| | Average | 2.2 | 2.6 | 33.7 | 65.1 |
| Liquid | 1 | 1.4 | 4.2 | 31.4 | 41.9 |
| | 2 | 1.6 | 2.9 | 16.3 | 41.2 |
| | 3 | 2.1 | 3.7 | 17.1 | 36.9 |
| | Average | 1.7 | 3.6 | 21.6 | 40.0 |

### Example 13

Effect of Different Cytokinins during Bud Induction on Corn Explant Transformation

An experiment was conducted to evaluate the effect of using different cytokinins during bud induction on corn explant transformation. Corn explants were surface sterilized, purified, rehydrated, inoculated with Agrobacterium, co-cultured, and subjected to the bud induction (without the second extended bud induction step) and regeneration steps as described in Example 1, unless otherwise noted. Glyphosate was used as the selection agent.

In this experiment, bud induction using four cytokinins, 6-Benzylaminopurine (BAP), Zeatin, thidiazuron (TDZ) and 6-(γ,γ-dimethylallylamino)purine (2iP), was evaluated. As shown in Table 30, bud induction using BAP at 10 mg/L or TDZ at 2 mg/L demonstrated significantly higher normal shoot frequencies compared to bud induction using 2 iP at 10 mg/L or zeatin at 2 mg/L. In this experiment, bud induction using TDZ produced a slightly higher normal shoot frequency compared to bud induction using BAP. In other studies that utilized, for instance, 1-8 mg/L BAP bud induction was also observed.

TABLE 30
Effect of different cytokinins during bud
induction on corn explant transformation.

| | | | Total | Shoot | # | |
|---|---|---|---|---|---|---|
| | Concentration | # | # | Frequency | Normal | Normal Shoot |
| Cytokinin | (mg/L) | Explants | Shoot | (%) | Shoot | Frequency (%) |
| BAP | 10 | 5,000 | 290 | 5.80 | 78 | 1.56 |
| 2iP | 10 | 5,000 | 7 | 0.14 | 3 | 0.06 |
| Zeatin | 2 | 5,000 | 10 | 0.20 | 3 | 0.06 |
| TDZ | 2 | 5,000 | 444 | 8.88 | 85 | 1.70 |

The shoot frequency was calculated as the number of inoculated explants producing shoots divided by the total number of inoculated explants. The normal shoot frequency was calculated as the number of normal shoots divided by the total number of inoculated explants.

### Example 14

Co-Culture without 2,4-D and Silwet® Improves Agrobacterium-Mediated Transformation of Wheat Seed Excised Explants

In this experiment, the effect of 2,4-D and Silwet®, a surfactant, in the co-culture medium for Agrobacterium-mediated transformation of wheat seed excised explants was evaluated. Wheat explants were surface sterilized, purified, inoculated with Agrobacterium, co-cultured, and subjected to delay, selection, and regeneration as described in Example 2, unless otherwise noted.

In this experiment, the addition of 2,4-D and Silwet® did not have a statistically significant effect on transformation frequency or normal plant frequency. As shown in Table 31, however, transformation frequency and normal plant frequency decreased when co-culture medium containing both 2,4-D and Silwet® was used compared to co-culture medium containing neither 2,4-D or Silwet®. Co-culture medium containing only Silwet® resulted in a higher normal plant frequency compared to co-culture medium containing neither Silwet® nor 2,4-D, however, transformation frequency was the highest in the absence of Silwet® and 2,4-D.

TABLE 31
Effect of 2,4-D and Silwet ® in co-culture
medium on wheat explant transformation.

| | | Transformation Frequency | Normal Plant Frequency |
|---|---|---|---|
| | | (%) ± Standard | (%) ± Standard |
| 2,4-D | Silwet ® | Deviation | Deviation |
| + | + | 1.0 ± 0.9 | 19.0 ± 11.3 |
| − | + | 5.0 ± 1.0 | 31.4 ± 2.0 |
| − | − | 7.1 ± 1.6 | 25.3 ± 5.0 |

Transformation frequency was calculated as the number of inoculated explants producing transgenic plants divided by the total number of inoculated explants. The normal plant frequency was calculated as the number of normal plants divided by the total number of plants regenerated.

### Example 15

Co-Culture without 2,4-D Improves Agrobacterium-Mediated Transformation of Corn Seed Excised Explants

Seventeen independent experiments were conducted to evaluate the effect of 2,4-D in the co-culture medium on corn explant transformation. Corn explants were surface sterilized, purified, rehydrated, inoculated with Agrobacterium, co-cultured, and subjected to bud induction, extended bud induction, and regeneration as described in Example 1, unless otherwise noted. Agrobacterium containing the same Ti plasmid as described in Example 9 was used for all experiments, except for experiments 13-17, in which two different bacterial origins of replication in the Ti-plasmid were evaluated in the presence or absence of 2,4-D in co-culture medium. In these cases, the Ti plasmids carried the same two expression cassettes flanked by T-DNA borders, one comprising the chloroplast-targeted CP4 EPSPS gene under the control of a constitutive promoter and the other comprising the uidA gene under the control of a similar constitutive promoter.

Table 32 provides a summary of results from treatments with and without 2,4-D in the co-culture medium from the 17 experiments. While these experiments included additional variables apart from the presence or absence of 2,4-D, the results show that removal of 2,4-D from the co-culture medium consistently resulted in higher normal plant frequencies in 16 out of 17 experiments across different sites and with different laboratory researchers, independent of any additional parameters. The increase in normal plant frequency ranged from 1.1 to 3.0-fold with an average increase of 1.8-fold.

TABLE 32
Removal of 2,4-D from co-culture media
improves corn explant transformation.

| | Normal Plant Frequency (%) | | |
|---|---|---|---|
| Experiment | With 2,4-D (5 mg/L) | Without 2,4-D | Fold Increase |
| 1 | 1.40 | 3.60 | 2.6 |
| 2 | 1.00 | 2.90 | 2.9 |
| 3 | 1.56 | 1.72 | 1.1 |
| 4 | 1.10 | 1.24 | 1.1 |
| 5 | 1.24 | 1.44 | 1.2 |
| 6 | 1.48 | 2.76 | 1.9 |
| 7 | 0.20 | 0.48 | 2.4 |
| 8 | 0.60 | 0.88 | 1.5 |
| 9 | 2.40 | 5.50 | 2.3 |
| 10 | 1.70 | 3.50 | 2.1 |
| 11 | 4.20 | 1.80 | 0.4 |
| 12 | 1.50 | 3.80 | 2.5 |
| 13 | 1.70 | 3.10 | 1.8 |
| 14 | 0.40 | 0.60 | 1.5 |
| 15 | 0.70 | 1.20 | 1.7 |
| 16 | 0.80 | 3.90 | 4.9 |
| 17 | 0.40 | 1.20 | 3.0 |
| OVERALL: | 1.32 | 2.33 | 1.8 |

The normal plant frequency was calculated as the number of normal plants divided by the total number of inoculated explants.

### Example 16

### Effect of Co-Culture Duration on Corn Explant Transformation

To evaluate the effect of co-culture duration on corn explant transformation, corn seed excised explants were surface sterilized in 70% ethanol for 4 minutes. Following incubation in rehydration medium comprising ⅖ strength of B5 macro salts except for CaCl₂, which is ½ strength, 1/10 of B5 micro salts and vitamins, 1 g/L potassium nitrate, 30 g/L dextrose, 2.8 mg/L sequestrene, 3.9 g/L 2-(N-morpholino)ethanesulfonic acid (MES), and 0.03 g/L Clearys 3336 WP, pH 5.4 on a shaker at 100 rpm at room temperature for 2 hours, the explants were inoculated with Agrobacterium as described in Example 1. The inoculated explants were then transferred to co-culture plates and co-cultured for 5 or 7 days before they were subjected to bud induction, extended bud induction, and regeneration as described in Example 1, except in this experiment the regeneration medium contained MS salts, B5 vitamins, 30 g/L sucrose, 0.69 g/L proline, 2 mg/L glycine, 1 g/L MES, 400 mg/L carbenicillin, 200 mg/L cefotaxime, 100 mg/L timentin and 20 μM glyphosate, pH 5.8. Regenerated shoots were rooted on rooting medium containing MS salts, B5 vitamins, 30 g/L sucrose, 0.69 g/L proline, 2 mg/L glycine, 1 g/L MES, 3 g/L Gelzan CM, 0.25 mg/L IBA, 250 mg/L carbenicillin, and 50 μM glyphosate, pH 5.8.

As shown in Table 33, co-culture for 7 days produced a higher total number of transgenic shoots compared to co-culture for 5 days, however, a higher percentage of the shoots produced were chimeric. As a result, co-culture for 5-days yielded a higher frequency of normal plants.

TABLE 33
Comparison of co-culture duration (5 vs
7 days) on corn explant transformation.

| | | | Shoot | | Normal Plant |
|---|---|---|---|---|---|
| Duration | # | Total # | Frequency | # Normal | Frequency |
| (Days) | Explants | Shoots | (%) | Plants | (%) |
| 5 | 1219 | 138 | 11.32 | 55 | 4.5 |
| 7 | 1250 | 147 | 11.80 | 37 | 3.0 |
| 5 | 1188 | 161 | 13.50 | 59 | 5.0 |
| 7 | 1219 | 160 | 13.10 | 32 | 2.6 |
| 5 | 1250 | 101 | 8.00 | 26 | 2.1 |
| 7 | 1250 | 127 | 10.20 | 33 | 2.6 |

The shoot frequency was calculated as the number of inoculated explants producing shoots divided by the total number of inoculated explants. The normal plant frequency was calculated as the number of normal plants divided by the total number of inoculated explants.

In a separate experiment, co-culture durations of 4 days and 5 days were evaluated in combination with the presence or absence of 5 mg/L 2,4-D in the co-culture medium. Corn seed excised explants were surface sterilized, rehydrated and inoculated with an Agrobacterium suspension as described in the preceding experiment. The Agrobacterium contained a chloroplast-targeted CP4 EPSPS gene under the control of a constitutive promoter flanked by T-DNA borders on the Ti plasmid. The inoculated explants were transferred to co-culture plates with or without 5 mg/L 2,4-D and co-cultured for 4 or 5 days prior to bud induction, extended bud induction, and regeneration as described in the preceding experiment.

As shown in Table 34, an average normal plant frequency of 2.47% was obtained when 2,4-D was absent in co-culture medium, as compared to 0.73% when 2,4-D was present. This data is consistent with the results described in Example 15. Furthermore, co-culture for 4 days produced a higher normal plant frequency compared to co-culture for 5 days. Table 35 provides average calculations based on the data provided in Table 34.

TABLE 34
Effect of 2,4-D in co-culture and co-culture
duration on corn explant transformation.

| | | | | | | Normal |
|---|---|---|---|---|---|---|
| 2,4-D | Dura- | | Total | Shoot | # | Plant |
| in Co- | tion | # | # | Frequency | Normal | Frequency |
| culture | (Days) | Explants | Shoots | (%) | Plants | (%) |
| − | 4 | 2132 | 45 | 2.1 | 29 | 1.4 |
| + | 4 | 2296 | 35 | 1.5 | 19 | 0.8 |
| − | 4 | 492 | 35 | 7.1 | 22 | 4.5 |
| − | 5 | 1148 | 27 | 2.4 | 17 | 1.5 |
| + | 4 | 1148 | 21 | 1.1 | 10 | 0.9 |
| + | 5 | 820 | 9 | 1.1 | 4 | 0.5 |

The shoot frequency was calculated as the number of inoculated explants producing shoots divided by the total number of inoculated explants. The normal plant frequency was calculated as the number of normal plants divided by the total number of inoculated explants.

TABLE 35
Removal of 2,4-D from co-culture media improves
corn explant transformation as demonstrated
by the average normal plant frequency (%).

| Duration | 2,4-D Present in | 2,4-D Absent in |
|---|---|---|
| (Days) | Co-Culture | Co-Culture |
| 4 | 0.85 | 2.95 |
| 5 | 0.5 | 1.50 |

The normal plant frequency was calculated as the number of normal plants divided by the total number of inoculated explants.

### Example 17

Agrobacterium-Mediated Transformation of Setaria viridis Crushed Seeds

Setaria viridis (green foxtail millet) can be an attractive model plant system for studying C₄ grass biology and other agronomic traits due to several characteristics, such as small stature (10-30 cm), rapid life cycle (6-9 weeks), prolific seed production (˜13,000 seeds per plant), self-compatibility, small genome size (˜395 Mb), diploid genetics (2n=18), and simple growth requirements. Agrobacterium-mediated transformation methods have been reported for S. viridis including tissue culture-based and in planta floral dip methods. See, e.g., Nguyen et al., “Robust and reproducible Agrobacterium transformation system of C4 genetic model species Setaria virdis”, Front. Plant Sci. 13:281 (2020); and Van Eck, J., “The Status of Setaria virdis Transformation: Agrobacterium-mediated to Floral Dip”, Front. Plant Sci. 9:652 (2018). However, prior tissue culture-based methods involve Agrobacterium transformation of seed-derived callus culture, which is labor-intensive and prone to somaclonal variation and off-types in plants. Non-tissue culture based floral dip methods involve exposure of immature inflorescences of S. viridis plants to Agrobacterium followed by recovery of mature seeds and identification of transgenic plants grown from those harvested seeds. However, in planta floral dip methods require growth chamber or greenhouse space for growing and maintaining the plants before and after Agrobacterium infection.

Described herein is a novel approach for Agrobacterium-mediated transformation of Setaria seeds. An initial attempt was made to develop an Agrobacterium-mediated transformation system using dry excised seed embryo explants from Setaria viridis seeds, similar to the excised corn and wheat embryo explant systems described in the Examples above. However, while potentially capable of being transformed and regenerated into plants, the excised Setaria seed embryos were very small and difficult to handle. Thus, alternative approaches were developed using whole or crushed Setaria viridis seeds for Agrobacterium-mediated transformation, instead of excised seed embryo explants. Transformed Setaria plants were cultured and regenerated into plants from the inoculated whole or crushed seeds using a bud induction and regeneration protocol similar to the systems and methods described herein for corn and wheat seed embryo explant culture and regeneration.

### Setaria Crushed Seed Preparation

Up to 10 ml of Setaria seeds (˜5-10 grams) were placed into a 50 ml Falcon tube containing 30 ml of 70% ethanol for sterilization. After shaking, the seeds were poured into a 200 ml to 400 ml disposable plastic beaker and incubated for 2 min. The ethanol was then removed with a 35 ml pipet. The total ethanol contacting time may preferably be controlled to less than 4 minutes for Setaria seeds. The seeds were then rinsed 3 times with −200 ml of sterile water. Alternatively, the seeds were sterilized with 10% bleach plus 0.1% Tween 20 for 3 minutes, followed by rinsing with sterile water 3 times. The sterilized seeds were soaked in sterile water for at least 3 hours to rehydrate and soften the seeds before the rolling and crushing step. The sterilized and rehydrated seeds were then transferred to a clean plastic sheet, spread into a single layer, and covered with a second plastic sheet. The seeds between the two sheet layers were then crushed by rolling a 1 L bottle or rolling pin until a majority of the seed coats were crushed or opened. The crushed seeds were then transferred into one or more 50 ml Falcon tubes.

### Agrobacterium Inoculum Preparation

Approximately 0.5 mL of thawed Agrobacterium glycerol stock was inoculated into 200 ml of liquid LB medium containing 50 mg/L spectinomycin and 30 mg/L gentamicin selection in a sterile 1 L flask. The flask was placed into an orbital shaker/incubator set to 200 rpm and cultured at 28° C. in the dark for 16-24 hours, or until the absorbance at 660 nm (OD₆₆₀) of the inoculum was within a range of 0.6-1.0. Following centrifugation of the overnight Agrobacterium culture at approximately 3,000 rpm or 2,620×g (Sorvall® 3B, 6000A rotor) at 4° C. for 25 minutes, the pellet was resuspended in an inoculation medium comprising ⅖ strength of B5 macro salts, 1/10 of B5 micro salts and vitamins, 1 g/L potassium nitrate, 30 g/L dextrose, and 3.9 g/L MES, 5 mg/L 2,4-D and 0.05% Silwet, to a final OD₆₆₀ of about 0.5. Even though an auxin (2,4-D) and surfactant was used in the inoculation medium, one or both of these components may alternatively be absent from the inoculation medium.

### Agrobacterium Inoculation and Co-Culture

About 30 ml of the Agrobacterium suspension from the Agrobacterium Inoculum Preparation step was added to the Falcon tubes with the crushed seeds from the Setaria Crushed Seed Preparation step. The Agrobacterium/crushed seed mixtures were then centrifuged in a Sorvall 5B at ˜690-1500×g at 4° C. for 30 minutes. Following centrifugation, the tubes were agitated to resuspend the crushed seeds, the resuspended crushed seeds were poured into a container, and most of the Agrobacterium suspension was removed by pipetting.

The inoculated crushed seeds (from about 200-300 of starting seeds) were transferred to a deep petri dish containing a piece of sterile Whatman #1 filter paper (82 mm) wetted with 1.25 ml of the inoculation medium described in Agrobacterium Inoculum Preparation step above and spread evenly into a single layer. The co-culture plates were incubated at 23° C. and about 70% relative humidity with a photoperiod of 16 hours light/8 hours dark for 3-8 days.

### Selection and Shoot/Root Regeneration

Following co-culture, the filter paper with the crushed seeds was transferred onto a shoot/bud induction medium comprising MS basal salts, B5 vitamins, 2 mg/L glycine, 690 mg/L proline, 1 g/L casein hydrolysate, 30 g/L sucrose, 1 g/L MES, 1 mg/L 2, 4-D, 10 mg/L BAP, 200 mg/L carbenicillin, 100 mg/L ticarcillin, 200 mg/L cefotaxime, and 3.5 g/L agarose, pH 5.8, with or without 10 μM glyphosate selection. Although no auxin was use in the shoot/bud induction medium in these experiments, low amounts of auxin could alternatively be used. The culture plates were incubated in a Percival incubator set at 28° C. with a photoperiod of 16 hours light/8 hours dark. Swelling and elongating seed explants were transferred to either a first regeneration medium comprising MS basal salts, B5 vitamins, 2 mg/L glycine, 690 mg/L proline, 30 g/L sucrose, 1 g/L MES, 400 mg/L carbenicillin, 100 mg/L ticarcillin, 200 mg/L cefotaxime, 30 μM glyphosate, and 3.5 g/L agarose, pH 5.8; or a second regeneration medium comprising MS basal salts, MS vitamins, 690 mg/L proline, 30 g/L sucrose, 1 g/L MES, 400 mg/L carbenicillin, 100 mg/L ticarcillin, 200 mg/L cefotaxime, 30 μM glyphosate, and 3.5 g/L agarose, pH 5.8. In experiment 3, after 4 weeks on the first regeneration medium, green multiple shoots were subcultured onto the same regeneration medium with 50 μM glyphosate. Dark green shoots were observed on this medium 2-3 weeks after the subculture and were transferred to rooting medium (the same medium or with low level of an auxin such as about 0.1-0.2 mg/L IAA). Although an auxin may have been used in the rooting medium in some of these experiments, the rooting medium may not contain any auxin or cytokinin.

In a first transformation experiment (Experiment 1), crushed seeds were inoculated with Agrobacterium tumefaciens cells containing a first Ti plasmid construct comprising two expression cassettes, the first encoding β-glucuronidase (GUS) as a scorable marker, and the second encoding CP4 EPSPS as a selection marker. The uidA (GUS) gene contained the second intron from the LS1 (light inducible gene) of potato and was under the control of a rice actin 1 promoter, an enhancer of duplicated 35S A1-B3 domain from the CaMV, the 5′ untranslated leader of wheat major chlorophyll a/b-binding protein, an intron from the rice actin 1 gene, and the 3′ UTR of the potato proteinase inhibitor II gene which functions to direct polyadenylation of the mRNA. The aroA gene encoding the CP4 EPSPS protein was under the control of the promoter, leader, and intron of the rice actin 1 gene, and the 3′UTR of the nopaline synthase gene of Agrobacterium tumefaciens Ti plasmid which functions to direct polyadenylation of the mRNA. The EPSPS protein was targeted to the chloroplast by a fused transit peptide from an Arabidopsis EPSPS gene. Two additional experiments (Experiments 2 and 3) were performed using Agrobacterium tumefaciens cells carrying a second Ti plasmid construct comprising the same two GUS and EPSPS expression cassettes as in the first Ti plasmid construct. Approximately 5 grams of Setaria seeds were used for each experiment.

Table 36 summarizes the experimental details and results from all three transformation experiments with crushed seeds. In Experiment 1, the crushed seeds were subjected to 1,500×g of centrifugal force during the inoculation step, co-cultured for 8 days, transferred to shoot/bud induction medium for about 21 days without selection, and then regenerated in the second regeneration medium with 30 μM of glyphosate selection for about 6 weeks. In Experiment 2, the crushed seeds were subjected to 800×g of centrifugal force during the inoculation step, co-cultured for 4 days, transferred to shoot/bud induction medium for about 17 days with 10 μM of glyphosate selection, and then regenerated in the first regeneration medium with 30 μM of glyphosate selection for about 5 weeks. In Experiment 3, the crushed seeds were subjected to 800×g of centrifugal force during the inoculation step, co-cultured for 8 days, transferred to shoot/bud induction medium for about 23 days with 10 μM of glyphosate selection, and then subjected to a first regeneration step in the first regeneration medium with 30 μM of glyphosate selection for about 4 weeks, followed by a second regeneration step in the first regeneration medium with 50 μM of glyphosate selection for about 3 weeks. One transgenic (GUS+) plant was obtained from Experiment 1, and three transgenic (GUS+) plants were obtained from Experiments 2 and 3.

TABLE 36
Summary of Agrobacterium-mediated transformation of crushed Setaria seeds.

| | | Centrifugal | | | | | | |
|---|---|---|---|---|---|---|---|---|
| | | Force | Co- | | | | | |
| | | during | culture | Shoot Induction | | Regeneration | | |
| | | Inoculation | Duration | Glyphosate | Duration | Glyphosate | Duration | # GUS + |
| Expt | Construct | (g) | (days) | Selection | (days) | Selection | (weeks) | Shoots |
| 1 | 1 | 1,500 | 8 | No | 21 | Second | 6 | 1 |
| | | | | | | Regeneration | | |
| | | | | | | Medium + | | |
| | | | | | | 30 μM | | |
| 2 | 2 | 800 | 4 | 10 μM | 17 | First | 5 | 2 |
| | | | | | | Regeneration | | |
| | | | | | | Medium + | | |
| | | | | | | 30 μM | | |
| 3 | 2 | 800 | 8 | 10 μM | 23 | 1) First | 4 | 1 |
| | | | | | | Regeneration | | |
| | | | | | | Medium + | | |
| | | | | | | 30 μM | | |
| | | | | | | 2) First | 3 | |
| | | | | | | Regeneration | | |
| | | | | | | Medium + | | |
| | | | | | | 50 μM | | |

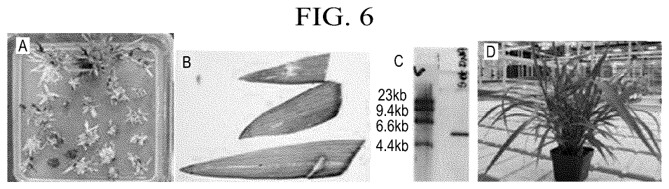

Images of an explant and regenerated plant with a transgenic insertion from the Experiment 1 is also shown in FIG. 6. Eight weeks after inoculation, a bushy shoot clump was obtained and confirmed by GUS staining (FIG. 6, Panel A and FIG. 6, Panel B). The regenerated plants were grown in the greenhouse (FIG. 6, Panel D) and seeds were harvested. Southern blot hybridization showed that the event contained a single copy of the transgene when probed with CP4 (FIG. 6, Panel C).

### Example 18

Agrobacterium-Mediated Direct Transformation of Setaria viridis Whole Seeds

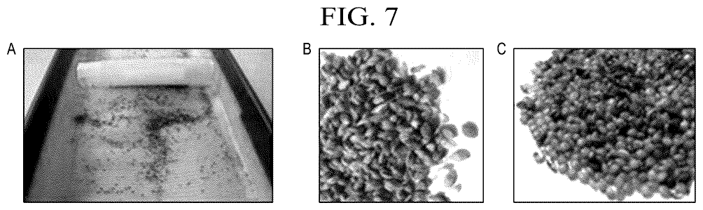

There are advantages to transforming whole or intact seeds without the need for crushing seeds or excising embryo explants. To test this possibility, whole or intact seeds were used for transformation with only their seed coats removed. The seed coats were removed from the Setaria seeds using a home-made polishing tool comprising a plastic drawer liner placed in the bottom of a tray and a paperboard core wrapped with another piece of ridged plastic liner. The Setaria seeds were placed on the liner in the tray and rolled over with the liner-covered paperboard core to loosen the seed coat mechanically (see FIG. 7, Panel A). FIG. 7, Panel B shows the Setaria seeds before removal of the seed coat, and FIG. 7, Panel C shows the Setaria seeds after removal or separation of the seed coats. The seeds were then poured into another container and blown with air to remove the seed coats, or the seeds were transferred into a water bath to allow for the seed to be separated from the seed coat as a result of the seed coat floating to the top of the water. Seeds were then collected for use in transformation.

After seed coat removal, the seeds were sterilized with 10% bleach plus 0.1% Tween 20 for 3 minutes, followed by rinsing with sterile water 3 times as described above in Example 17. The intact seeds were inoculated with Agrobacterium tumefaciens cells with the second Ti plasmid construct and cultured as described above for the crushed seeds in Example 17, except that the BAP concentration in the shoot/bud induction medium was 4 mg/L. Although no auxin was use in the shoot/bud induction medium in these experiments, low amounts of auxin could alternatively be used. Approximately 5 grams of Setaria seeds were used for each experiment. Plants were then regenerated from the inoculated and cultured seeds.

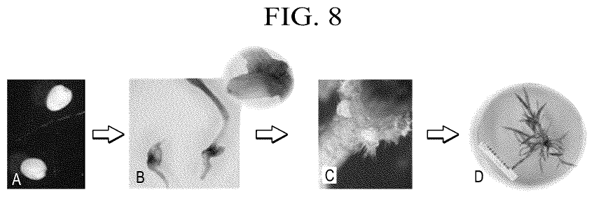

FIG. 8 shows the general progression of transformation and shoot regeneration after Agrobacterium infection of whole or intact seeds. Table 37 summarizes the experimental details and results from all three of these transformation experiments (Experiments 4, 5 and 6) with intact seeds. In Experiment 4, the seeds were subjected to 800×g of centrifugal force during the inoculation step, co-cultured for about 4 days, transferred to shoot/bud induction medium for about 8 days with 10 μM of glyphosate selection, and then regenerated in the first regeneration medium with 30 μM of glyphosate selection for about 6 weeks. In Experiment 5, the seeds were subjected to 1,500×g of centrifugal force during the inoculation step, co-cultured for 4 days, transferred to shoot/bud induction medium for about 10 days with 10 μM of glyphosate selection, and then regenerated in the first regeneration medium with 30 μM of glyphosate selection for about 5 weeks. In Experiment 6, the seeds were subjected to 1,500×g of centrifugal force during the inoculation step, co-cultured for 8 days, transferred to shoot/bud induction medium for about 10 days with 10 μM of glyphosate selection, and then subjected to a first regeneration step in the first regeneration medium with 30 μM of glyphosate selection for about 3 weeks, followed by a second regeneration step in the first regeneration medium with 50 μM of glyphosate selection for about 3 weeks. Two transgenic (GUS+) plants were obtained from Experiment 4, one transgenic (GUS+) plant was obtained from Experiment 5, and one transgenic (GUS+) plant was obtained in each Experiment 5 and 6.

TABLE 37
Summary of Agrobacterium-mediated transformation of Setaria whole seeds.

| | | Centrifugal | | | | | | |
|---|---|---|---|---|---|---|---|---|
| | | Force | Co- | | | | | |
| | | during | culture | Shoot Induction | | Regeneration | | |
| | | Inoculation | Duration | Glyphosate | Duration | Glyphosate | Duration | # GUS + |
| Expt | Construct | (g) | (days) | Selection | (days) | Selection | (weeks) | Shoots |
| 4 | 2 | 800 | 4 | 10 μM | 8 | First | 6 | 2 |
| | | | | | | Regeneration | | |
| | | | | | | Medium + | | |
| | | | | | | 30 μM | | |
| 5 | 2 | 1,500 | 4 | 10 μM | 10 | First | 5 | 1 |
| | | | | | | Regeneration | | |
| | | | | | | Medium + | | |
| | | | | | | 30 μM | | |
| 6 | 2 | 1,500 | 8 | 10 μM | 10 | 1) First | 3 | 1 |
| | | | | | | Regeneration | | |
| | | | | | | Medium + | | |
| | | | | | | 30 μM | | |
| | | | | | | 2) First | 3 | |
| | | | | | | Regeneration | | |
| | | | | | | Medium + | | |
| | | | | | | 50 μM | | |

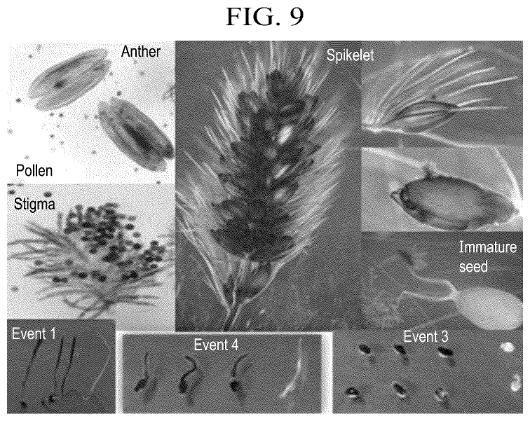

To confirm the transmission of the transgenes, R₀ reproductive tissues of the transformed plants and R₁ seedlings were stained for GUS expression. The staining results are shown in FIG. 9. GUS expression was observed in anthers, pollen stigma, spikelet, and immature seeds of transformed plants, as well as in germinated R₁ seedlings. R₁ seeds from two GUS-positive Setaria plant events were analyzed for segregation: Event 1 was from transformation of whole seeds according to the present Example 18, and Event 4 was from transformation of crushed seeds according to Example 17. Seedlings germinated from these R₁ seeds were stained for GUS expression. As shown in Table 38, both events showed an approximate 3:1 segregation, indicating that the transgene was integrated into a single locus.

TABLE 38
Transgene transmission in R1 progenies
of the transgenic Setaria plants.

| | R₀ | #GUS Positive | #GUS Negative | Chi Square |
|---|---|---|---|---|
| | Phenotype | Seedlings | Seedlings | Test |
| Event 1 | normal | 225 | 79 | ~3:1 |
| Event 4 | normal | 111 | 41 | ~3:1 |

The results provided in Examples 17 and 18 support the use of whole or intact monocot seeds or crushed or partially opened monocot seeds for use in transformation and generation of genetically modified monocot plants through a multiple shoot/bud induction and regeneration process. It is demonstrated herein that transgenic Setaria verdis plants can be obtained by direct transformation of whole or crushed seeds using Agrobacterium-mediated transformation. The use of whole, intact, crushed or partially opened seeds could greatly facilitate the transformation of monocot plants by avoiding the need for embryo explant excision.

1. A method of producing a genetically modified monocot plant or plant part comprising:
introducing a heterologous polynucleotide molecule into at least one cell of a monocot seed embryo explant comprising meristematic tissue by inoculating the embryo explant with an inoculation medium comprising a Rhizobiales bacterium competent to transform said at least one cell with the heterologous polynucleotide molecule;
culturing the embryo explant in contact with a first bud induction medium comprising a first auxin and a first cytokinin at a temperature of about 33° C. to about 37° C. for a first time period followed by culturing the embryo explant in contact with the first bud induction medium at a temperature of about 26° C. to about 30° C. for a second time period, wherein the concentration of the first cytokinin in the first bud induction medium is about 5 mg/L to about 15 mg/L and the concentration of the first auxin in the first bud induction medium is about 0.5 mg/L to about 1.5 mg/L; and
regenerating the genetically modified monocot plant or plant part from said embryo explant.

2. The method of claim 1, wherein the first time period or the second time period is about 2 days to about 14 days or about 6 days to about 8 days.

3. The method of claim 1, wherein:
the first cytokinin is selected from the group consisting of: 6-benzylaminopurine (BAP), thidiazuron (TDZ), kinetin, zeatin, diphenyl urea (DPU), 6-(gamma,gamma-dimethylallylamino)purine (2iP), and meta-topolin; or
the first auxin is selected from the group consisting of: 2,4-dichlorophenoxy-acetic acid (2,4-D), 4-amino-3,5,6-trichloro-picolinic acid (picloram), indole-3-acetic acid (IAA), indole-3-butyric acid (IBA), naphthalene acetic acid (NAA), 4-chlorophenoxy acetic acid or p-chloro-phenoxy acetic acid (4-CPA or pCPA), 2,4,5-trichloro-phenoxy acetic acid (2,4,5-T), 2,3,5-triiodobenzoic acid (TIBA), phenylacetic acid (PAA), and 3,6-dichloro-2-methoxy-benzoic acid (dicamba).

4. The method of claim 1, wherein the first cytokinin is 6-benzylaminopurine (BAP) and the first auxin is 2,4-dichlorophenoxy-acetic acid (2,4-D).

5. The method of claim 1, the method further comprising culturing the embryo explant in contact with a second bud induction medium comprising the first auxin or a second auxin and the first cytokinin or a second cytokinin, wherein the culturing of the embryo explant in contact with the second bud induction medium is performed after the embryo explant is cultured in contact with the first bud induction medium and before regenerating the genetically modified monocot plant or plant part from the cultured embryo explant in contact with the regeneration medium.

6. The method of claim 5, wherein:
the embryo explant is cultured in contact with the second bud induction medium for about 4 days to about 28 days or about 7 to about 14 days; or
the embryo explant is cultured in contact with the second bud induction medium at a temperature of about 20° C. to about 32° C., about 25° C. to about 29° C., or about 27° C. to about 28° C.

7. The method of claim 5, wherein the second bud induction medium comprises a high cytokinin to auxin ratio.

8. The method of claim 1, wherein the genetically modified monocot plant is a corn plant, a wheat plant, a rice plant, a barley plant, a turfgrass plant, or a sorghum plant.

9. The method of claim 1, the method further comprising:
co-culturing the embryo explant with the Rhizobiales bacterium in contact with a co-culture medium; or
applying a force treatment to the embryo explant in the inoculation medium or prior to inoculating the embryo explant with the inoculation medium.

10. A method of producing a genetically modified monocot plant or plant part comprising:
introducing a heterologous polynucleotide molecule into at least one cell of a monocot seed embryo explant comprising meristematic tissue by inoculating the embryo explant with an inoculation medium comprising a Rhizobiales bacterium competent to transform said at least one cell with the heterologous polynucleotide molecule;
applying a pressure treatment and a gravitational force treatment to the embryo explant;
culturing the embryo explant in contact with a first bud induction medium comprising a first auxin and a first cytokinin at a temperature of about 33° C. to about 37° C.; and
regenerating the genetically modified monocot plant or plant part from said embryo explant.

11. The method of claim 10, the method comprising:
applying the pressure treatment and the gravitational force treatment to the embryo explant in contact with the inoculation medium;
applying the pressure treatment and the gravitational force treatment to the embryo explant prior to inoculating the embryo explant with the inoculation medium;
applying the pressure treatment before the gravitational force treatment;
applying the gravitational force treatment before the pressure treatment;
applying the gravitational force treatment to the embryo explant for about 1 minute to about 2 hours, about 2 minutes to about 110 minutes, about 5 minutes to about 90 minutes, about 10 minutes to about 60 minutes, or about 20 minutes to about 40 minutes; or
applying the pressure treatment to the embryo explant for about 10 seconds to about 10 minutes, about 15 seconds to about 8 minutes, about 30 seconds to about 6 minutes, or about 2 minutes to about 4 minutes.

12. The method of claim 10, wherein:
applying the gravitational force treatment comprises applying about 100×g to about 10,000×g, about 500×g to about 3,000×g, about 655×g, or about 2620×g of force; or
applying the pressure treatment comprises applying about 100 psi to about 1,000 psi, about 125 psi to about 750 psi, about 150 psi to about 500 psi, about 200 psi to about 400 psi, or about 300 psi of force.

13. The method of claim 10, wherein the Rhizobiales bacterium is selected from the group consisting of a Rhizobiaceae bacterium, a Phyllobacteriaceae bacterium, a Brucellaceae bacterium, a Bradyrhizobiaceae bacterium, a Xanthobacteraceae bacterium, an Agrobacterium bacterium, a Rhizobium bacterium, a Sinorhizobium bacterium, a Mesorhizobium bacterium, a Phyllobacterium bacterium, an Ochrobactrum bacterium, a Bradyrhizobium bacterium, and an Azorhizobium bacterium.

14. The method of claim 10, wherein the OD₆₆₀ of Rhizobiales bacterium in the inoculation medium is about 0.5 to about 2.0, about 0.75 to about 1.25, or about 1.0.

15. The method of claim 10, the method further comprising:
co-culturing the embryo explant with the Rhizobiales bacterium in contact with a co-culture medium.

16. The method of claim 15, wherein the OD₆₆₀ of the Rhizobiales bacterium in the co-culture medium is about 0.5 to about 2.0, about 0.75 to about 1.25, or about 1.0.

17. The method of claim 10, wherein the genetically modified monocot plant is a corn plant, a wheat plant, a rice plant, a barley plant, a turfgrass plant, or a sorghum plant.

18. A method of producing a genetically modified monocot plant or plant part comprising:
introducing a heterologous polynucleotide molecule into at least one cell of a monocot seed embryo explant comprising meristematic tissue;
culturing the embryo explant in contact with a first bud induction medium comprising a first auxin and a first cytokinin; and
regenerating the genetically modified monocot plant or plant part from the cultured embryo explant in contact with a regeneration medium, wherein the regeneration medium comprises a total nitrogen concentration of about 0.5 mM to about 20 mM.

19. The method of claim 18, wherein the regeneration medium comprises:
a total salt concentration of less than about 2800 mg/L, less than about 2500 mg/L, or in a range from about 2200 mg/L to about 2500 mg/L;
a nitrate ion concentration of about 0.5 mM to about 20 mM;
an ammonium ion concentration of about 0.5 mM to about 15 mM;
a potassium ion concentration of about 0.5 mM to about 15 mM;
a sulfate ion concentration greater than or equal to about 5 mM;
an ammonium nitrate concentration of about 100 mg/L to about 1000 mg/L;
a calcium chloride concentration less than or equal to about 100 mg/L;
a calcium nitrate concentration less than or equal to about 500 mg/L; or
a potassium sulfate concentration greater than or equal to about 500 mg/L.

20. The method of claim 18, the method comprising:
regenerating the genetically modified monocot plant or plant part at a temperature of about 20° C. to about 32° C., about 25° C. to about 29° C., or about 27° C. to about 28° C.; or
regenerating the genetically modified monocot plant or plant part for about 20 days to about 50 days or about 28 days to about 42 days.

21. The method of claim 18, the method further comprising culturing the embryo explant in contact with a second bud induction medium comprising the first auxin or a second auxin and the first cytokinin or a second cytokinin, wherein the culturing of the embryo explant in contact with the second bud induction medium is performed after the embryo explant is cultured in contact with the first bud induction medium and before regenerating the genetically modified monocot plant or plant part from the cultured embryo explant in contact with the regeneration medium.

22. The method of claim 18, the method further comprising:
co-culturing the embryo explant with the Rhizobiales bacterium in contact with a co-culture medium; or
applying a force treatment to the embryo explant in the inoculation medium or prior to inoculating the embryo explant with the inoculation medium.

23. The method of claim 18, wherein the genetically modified monocot plant is a corn plant, a wheat plant, a rice plant, a barley plant, a turfgrass plant, or a sorghum plant.
